(19) United States
(12) Patent Application Publication
(10) Pub. No.: US 2023/0203052 A1
(43) Pub. Date: Jun. 29, 2023

(54) ACYCLIC CUCURBITURILS, METHODS OF MAKING SAME, AND USES THEREOF

(71) Applicant: University of Maryland, College Park, College Park, MD (US)

(72) Inventors: Lyle David ISAACS, Silver Spring, MD (US); Xiaoyong LU, Gaithersburg, MD (US)

(21) Appl. No.: 17/996,821

(22) PCT Filed: Apr. 20, 2021
(86) PCT No.: PCT/US2021/028252

§ 371 Date: Oct. 21, 2022

Related U.S. Application Data
(60) Provisional application No. 63/013,229, filed on Apr. 21, 2020.

(51) Int. Cl. C07D 487/22; A61K 47/54; A61P 21/00

(52) U.S. Cl. CPC C07D 487/22; A61K 47/547; A61P 21/00

(57) ABSTRACT

Disclosed herein are acyclic sulfated cucurbit[n]uril containing sulfate substituent(s), compositions containing the same, and method of preparation thereof. These compounds are useful, for example, as sequestering agents for drugs of abuse.

### CROSS REFERENCE TO RELATED APPLICATIONS

This application claims priority to U.S. Provisional Application No. 63/013,229, filed on Apr. 21, 2020, the disclosure of which is hereby incorporated by reference.

### STATEMENT REGARDING FEDERALLY SPONSORED RESEARCH

This invention was made with government support under grant no. CHE-1404911 awarded by National Science Foundation. The government has certain rights in the invention.

### BACKGROUND OF THE DISCLOSURE

The preparation of molecular container compounds and their use in basic science and real world applications has been a focal point of research in supramolecular chemistry for several decades. For example, cyclodextrin molecular containers have practical real world use as solubilizing excipients for hydrophobic drugs, as the active ingredient in the household product Febreeze™, and as a sequestration agent for neuromuscular blockers in the form of Sugammadex. Cucurbit[n]uril molecular containers have become increasingly popular in the past decade due in part to their high affinity binding toward hydrophobic (di)cations (e.g., Kₐ commonly 10⁶ M⁻¹, often 10⁹ M⁻¹ and up to 10¹⁷ M⁻¹ in select cases) and their stimuli responsive host⋅guest binding properties. CB[n] hosts are thereby well suited as components of functional systems (e.g., sensing ensembles, drug delivery systems, and supramolecular materials). In recent years, we and others, have been exploring the synthesis and molecular recognition properties of acyclic CB[n]-type receptors (e.g., M1, FIG. 1) which retain the essential binding properties of macrocyclic CB[n] but are more easily functionalized. For example, M1 has been used as a solubilizing excipient for insoluble drugs and as in vivo sequestration agents for neuromuscular blockers (rocuronium, vecuronium, and cisatracurium) and drugs of abuse (e.g., methamphetamine and fentanyl). These applications require hosts with maximal binding affinities to allow them to outcompete the cognate biological receptors.

### SUMMARY OF THE DISCLOSURE

The present disclosure provides acyclic sulfated cucurbit[n]uril with sulfate substituent(s). Also described herein are compositions comprising acyclic sulfated cucurbit[n]uril with sulfate substituent(s), methods of making acyclic sulfated cucurbit[n]uril with sulfate substituent(s), and methods of using acyclic sulfated cucurbit[n]uril with sulfate substituent(s).

In an aspect, the present disclosure provides compounds. The compounds are acyclic sulfated cucurbit[n]urils having one or more sulfate substituents. The compounds comprise a plurality of linked glycoluril groups.

In various examples, a compound has the following structure:

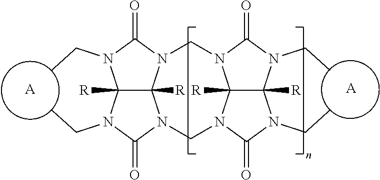

where each R is independently a hydrogen, a C₁ to C₂₀ alkyl group, a C₃ to C₂₀ carbocyclic group, a C₁ to C₂₀ heterocyclic group, a carboxylic acid group, a ester group, an amide group, a hydroxy, or an ether group. Optionally, adjacent R groups form a C₃ to C₂₀ carbocyclic ring or heterocyclic ring.

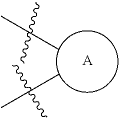

(or simply “A groups”)
is independently a C₅ to C₂₀ carbocyclic ring system or C₂ to C₂₀ heterocyclic ring system, where the ring system comprises one or more rings. At least one ring system has at least one solubilizing group selected from —OS(O)₂O⁻M⁺ and —OS(O)₂OH, where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and tris(hydroxymethyl)aminomethane (TRIS), and n is 0 to 6 (e.g., 1, 2, 3, 4, 5, 6). The compound may be a stereoisomer or mixture thereof, a salt, a partial salt, a hydrate, a polymorph or a mixture thereof of the above structure. In various embodiments, n is 3. In various embodiments, each R is independently hydrogen or methyl.

In an aspect, the present disclosure provides compositions comprising one or more compound(s). Non-limiting examples of compositions are described herein.

In an aspect, the present disclosure provides uses of acyclic sulfated cucurbit[n]urils. Non-limiting examples of uses of acyclic sulfated cucurbit[n]urils are provided herein, for example, non-limiting examples of uses of acyclic sulfated cucurbit[n]urils are described in the Statements and Examples.

In an aspect, the present disclosure provides articles comprising compounds of the present disclosure.

### BRIEF DESCRIPTION OF THE FIGURES

For a fuller understanding of the nature and objects of the disclosure, reference should be made to the following detailed description taken in conjunction with the accompanying figures.

FIG. 1 shows an acyclic CB[n]-type host 1.

FIG. 2 shows synthesis of host 1. Conditions: a) TFA, 25° C., N₂, 16 h; b) pyridine sulfur trioxide, pyridine, 90° C., N₂, 18 h.

FIG. 3 shows structures of guests 5-23 used herein.

FIG. 4 shows ¹H NMR spectra (D₂O, 600 MHz) recorded for: a) 1 (1 mM), b) a mixture of 1 (1 mM) and 6d (1 mM), c) a mixture of 1 (1 mM) and 6d (2 mM), and d) 6d (1 mM). Resonances for bound guests as marked with an asterisk (*).

FIG. 5 shows cross eyed stereoviews of the x-ray crystal structures of: a) 1⋅6d, and b) 1⋅6a. Carbon, hydrogen, nitrogen, oxygen, and hydrogen bonds are shown.

FIG. 6 shows a) thermogram recorded during the titration of a mixture of 1 (100 μM) and 13 (2 mM) in the cell with a solution of 6d (1.0 mM) in the syringe, and b) fitting of the data to a competition binding model to extract Kₐ=6.79×10⁹ M⁻¹ and ΔH=−12.1 kcal mol⁻¹.

FIG. 7 shows ¹H NMR spectra (600 MHz, D₂O, RT) recorded for compound 1.

FIG. 8 shows ¹H, ¹H DQCOSY NMR spectra (600 MHz, D₂O, RT) recorded for compound 1.

FIG. 9 shows ¹³C NMR spectra (600 MHz, D₂O, RT) recorded for compound 1.

FIG. 10 shows ¹³C DEPT135 NMR spectra (600 MHz, D₂O, RT) recorded for compound 1.

FIG. 11 shows ¹H NMR spectra (600 MHz, DMSO-d₆, RT) recorded for compound 4.

FIG. 12 shows ¹³C NMR spectra (125 MHz, DMSO-d₆, RT) recorded for compound 4.

FIG. 13 shows ¹H NMR spectra (600 MHz, D₂O, RT) recorded for 1 as a function of concentration.

FIG. 14 shows ¹H NMR spectra (600 MHz, D₂O, RT) recorded for a) host 1 (1.0 mM), b) host 1 (1.0 mM) and guest 5a (1.0 mM), c) host 1 (1.0 mM) and guest 5a (2.0 mM), d) guest 5a (1.0 mM).

FIG. 15 shows ¹H NMR spectra (600 MHz, D₂O, RT) recorded for a) host 1 (1.0 mM), b) host 1 (1.0 mM) and guest 6Q (1.0 mM), c) host 1 (1.0 mM) and guest 6Q (2.0 mM), d) guest 6Q (1.0 mM).

FIG. 16 shows ¹H NMR spectra (600 MHz, D₂O, RT) recorded for a) host 1 (1.0 mM), b) host 1 (1.0 mM) and guest 11d (1.0 mM), c) host 1 (1.0 mM) and guest 11d (2.0 mM), d) guest lid (1.0 mM).

FIG. 17 shows ¹H NMR spectra (600 MHz, D₂O, RT) recorded for a) host 1 (1.0 mM), b) host 1 (1.0 mM) and guest 11a (1.0 mM), c) host 1 (1.0 mM) and guest 11a (2.0 mM), d) 11a (1.0 mM).

FIG. 18 shows ¹H NMR spectra (600 MHz, D₂O, RT) recorded for a) host 1 (1.0 mM), b) host 1 (1.0 mM) and guest 6d (1.0 mM), c) host 1 (1.0 mM) and guest 6d (2.0 mM), d) guest 6d (1.0 mM).

FIG. 19 shows ¹H NMR spectra (600 MHz, D₂O, RT) recorded for a) host 1 (1.0 mM), b) host 1 (1.0 mM) and guest 6c (1.0 mM), c) host 1 (1.0 mM) and guest 6c (2.0 mM), d) guest 6c (1.0 mM).

FIG. 20 shows ¹H NMR spectra (600 MHz, D₂O, RT) recorded for a) host 1 (1.0 mM), b) host 1 (1.0 mM) and guest 6b (1.0 mM), c) host 1 (1.0 mM) and guest 6b (2.0 mM), d) guest 6b (1.0 mM).

FIG. 21 shows ¹H NMR spectra (600 MHz, D₂O, RT) recorded for a) host 1 (1.0 mM), b) host 1 (1.0 mM) and guest 6a (1.0 mM), c) host 1 (1.0 mM) and guest 6a (2.0 mM), d) guest 6a (1.0 mM).

FIG. 22 shows ¹H NMR spectra (600 MHz, D₂O, RT) recorded for a) host 1 (1.0 mM), b) host 1 (1.0 mM) and guest 7d (1.0 mM), c) host 1 (1.0 mM) and guest 7d (2.0 mM), d) guest 7d (1.0 mM).

FIG. 23 shows ¹H NMR spectra (600 MHz, D₂O, RT) recorded for a) host 1 (1.0 mM), b) host 1 (1.0 mM) and guest 8d (1.0 mM), c) host 1 (1.0 mM) and guest 8d (2.0 mM), d) guest 8d (1.0 mM).

FIG. 24 shows ¹H NMR spectra (600 MHz, D₂O, RT) recorded for a) host 1 (1.0 mM), b) host 1 (1.0 mM) and guest 9d (1.0 mM), c) host 1 (1.0 mM) and guest 9d (2.0 mM), d) guest 9d (1.0 mM).

FIG. 25 shows ¹H NMR spectra (600 MHz, D₂O, RT) recorded for a) host 1 (1.0 mM), b) host 1 (1.0 mM) and guest 10d (1.0 mM), c) host 1 (1.0 mM) and guest 10d (2.0 mM), d) guest 10d (1.0 mM).

FIG. 26 shows ¹H NMR spectra (600 MHz, D₂O, RT) recorded for a) host 1 (1.0 mM), b) host 1 (1.0 mM) and guest 12d (1.0 mM), c) host 1 (1.0 mM) and guest 12d (2.0 mM), d) guest 12d (1.0 mM).

FIG. 27 shows ¹H NMR spectra (600 MHz, D₂O, RT) recorded for a) host 1 (1.0 mM), b) host 1 (1.0 mM) and guest 12a (1.0 mM), c) host 1 (1.0 mM) and guest 12a (2.0 mM), d) guest 12a (1.0 mM).

FIG. 28 shows ¹H NMR spectra (600 MHz, D₂O, RT) recorded for a) host 1 (1.0 mM), b) host 1 (1.0 mM) and guest 13a (1.0 mM), c) host 1 (1.0 mM) and guest 13a (2.0 mM), d) guest 13a (1.0 mM).

FIG. 29 shows ¹H NMR spectra (600 MHz, D₂O, RT) recorded for a) host 1 (1.0 mM), b) host 1 (250 μM) and guest 19 (250 μM), c) host 1 (250 μM) and guest 19 (500 μM), d) guest 19 (500 μM).

FIG. 30 shows ¹H NMR spectra (600 MHz, D₂O, RT) recorded for a) host 1 (1.0 mM), b) host 1 (250 μM) and guest 20 (250 μM), c) host 1 (250 μM) and guest 20 (500 μM), d) guest 20 (500 μM).

FIG. 31 shows ¹H NMR spectra (600 MHz, D₂O, RT) recorded for a) host 1 (1.0 mM), b) host 1 (1.0 mM) and guest 22 (1.0 mM), c) host 1 (1.0 mM) and guest 22 (2.0 mM), d) guest 22 (1.0 mM).

FIG. 32 shows ¹H NMR spectra (600 MHz, D₂O, RT) recorded for a) host 1 (1.0 mM), b) host 1 (1.0 mM) and guest 23 (1.0 mM), c) host 1 (1.0 mM) and guest 23 (2.0 mM), d) guest 23 (1.0 mM).

FIG. 33 shows isothermal titration calorimetry (ITC) curve obtained through direct binding titration studies. A solution of 1 (100 μM) in the cell was titrated with 5a (1.00 mM) in the syringe at 298.0 K in 20 mM sodium phosphate buffered water at pH 7.4. Kₐ=1.68×10⁶ M⁻¹.

FIG. 34 shows isothermal titration calorimetry (ITC) curve obtained through competition binding studies. A solution of 1 (100 μM) and 5a (500 μM) in the cell was titrated with 6a (1.00 mM) in the syringe at 298.0 K in 20 mM sodium phosphate buffered water at pH 7.4. Kₐ=3.70×10⁸ M⁻¹.

FIG. 35 shows isothermal titration calorimetry (ITC) curve obtained through competition binding studies. A solution of 1 (100 μM) and 13a (500 μM) in the cell was titrated with 6b (1.00 mM) in the syringe at 298.0 K in 20 mM sodium phosphate buffered water at pH 7.4. Kₐ=5.26×10⁸ M⁻¹.

FIG. 36 shows isothermal titration calorimetry (ITC) curve obtained through competition binding studies. A solution of 1 (100 μM) and 13a (500 μM) in the cell was titrated with 6c (1.00 mM) in the syringe at 298.0 K in 20 mM sodium phosphate buffered water at pH 7.4. Kₐ=5.74×10⁸ M⁻¹.

FIG. 37 shows isothermal titration calorimetry (ITC) curve obtained through competition binding studies. A solution of 1 (100 μM) and 13a (2000 μM) in the cell was titrated with 6d (1.00 mM) in the syringe at 298.0 K in 20 mM sodium phosphate buffered water at pH 7.4. Kₐ=6.71×10⁹ M⁻¹.

FIG. 38 shows isothermal titration calorimetry (ITC) curve obtained through direct binding titration studies. A solution of 1 (10 μM) in the cell was titrated with 6Q (100 μM) in the syringe at 298.0 K in 20 mM sodium phosphate buffered water at pH 7.4. Kₐ=7.57×10⁶ M⁻¹.

FIG. 39 shows isothermal titration calorimetry (ITC) curve obtained through competition binding studies. A solution of 1 (100 μM) and 13a (2000 μM) in the cell was titrated with 7d (1.00 mM) in the syringe at 298.0 K in 20 mM sodium phosphate buffered water at pH 7.4. Kₐ=6.06×10⁹ M⁻¹.

FIG. 40 shows isothermal titration calorimetry (ITC) curve obtained through competition binding studies. A solution of 1 (100 μM) and 13a (500 μM) in the cell was titrated with 8d (1.00 mM) in the syringe at 298.0 K in 20 mM sodium phosphate buffered water at pH 7.4. Kₐ=1.75×10⁹ M⁻¹.

FIG. 41 shows isothermal titration calorimetry (ITC) curve obtained through competition binding studies. A solution of 1 (100 μM) and 13a (500 μM) in the cell was titrated with 9d (1.00 mM) in the syringe at 298.0 K in 20 mM sodium phosphate buffered water at pH 7.4. Kₐ=7.57×10⁸ M⁻¹.

FIG. 42 shows isothermal titration calorimetry (ITC) curve obtained through competition binding studies. A solution of 1 (100 μM) and 13a (500 μM) in the cell was titrated with 10d (1.00 mM) in the syringe at 298.0 K in 20 mM sodium phosphate buffered water at pH 7.4. Kₐ=5.43×10⁸ M⁻¹.

FIG. 43 shows isothermal titration calorimetry (ITC) curve obtained through direct binding studies. A solution of 1 (100 μM) in the cell was titrated with 12a (1.00 mM) in the syringe at 298.0 K in 20 mM sodium phosphate buffered water at pH 7.4. Kₐ=9.90×10⁵ M⁻¹.

FIG. 44 shows isothermal titration calorimetry (ITC) curve obtained through competition binding studies. A solution of 1 (50 μM) and 12d (1500 μM) in the cell was titrated with 6d (500 μM) in the syringe at 298.0 K in 20 mM sodium phosphate buffered water at pH 7.4. Kₐ=6.66×10⁶ M⁻¹.

FIG. 45 shows isothermal titration calorimetry (ITC) curve obtained through direct binding studies. A solution of 1 (10.0 μM) in the cell was titrated with 13a (100 μM) in the syringe at 298.0 K in 20 mM sodium phosphate buffered water at pH 7.4. Kₐ=3.41×10⁶ M⁻¹.

FIG. 46 shows isothermal titration calorimetry (ITC) curve obtained through competition binding studies. A solution of 1 (100 μM) and 5a (500 μM) in the cell was titrated with 11a (1.00 mM) in the syringe at 298.0 K in 20 mM sodium phosphate buffered water at pH 7.4. Kₐ=9.71×10⁸ M⁻¹.

FIG. 47 shows isothermal titration calorimetry (ITC) curve obtained through competition binding studies. A solution of 1 (100 μM) and 5a (500 μM) in the cell was titrated with 11d (1.00 mM) in the syringe at 298.0 K in 20 mM sodium phosphate buffered water at pH 7.4. Kₐ=1.05×10⁹ M⁻¹.

FIG. 48 shows isothermal titration calorimetry (ITC) curve obtained through direct binding studies. A solution of 1 (100.0 μM) in the cell was titrated with 23 (1.00 mM) in the syringe at 298.0 K in 20 mM sodium phosphate buffered water at pH 7.4. Kₐ=2.31×10⁵ M⁻¹.

FIG. 49 shows isothermal titration calorimetry (ITC) curve obtained through direct binding studies. A solution of 1 (100.0 μM) in the cell was titrated with 22 (1.00 mM) in the syringe at 298.0 K in 20 mM sodium phosphate buffered water at pH 7.4. Kₐ=2.41×10⁴ M⁻¹.

FIG. 50 shows isothermal titration calorimetry (ITC) curve obtained through competition binding studies. A solution of 1 (100 μM) and 5a (500 μM) in the cell was titrated with 19 (1.00 mM) in the syringe at 298.0 K in 20 mM sodium phosphate buffered water at pH 7.4. Kₐ=6.29×10⁸ M⁻¹.

FIG. 51 shows isothermal titration calorimetry (ITC) curve obtained through competition binding studies. A solution of 1 (100 μM) and 13a (500 μM) in the cell was titrated with 20 (1.00 mM) in the syringe at 298.0 K in 20 mM sodium phosphate buffered water at pH 7.4. Kₐ=1.00×10⁹ M⁻¹.

FIG. 52 shows isothermal titration calorimetry (ITC) curve obtained through direct binding studies. A solution of 1 (80.0 μM) in the cell was titrated with 21 (500.0 μM) in the syringe at 298.0 K in 20 mM sodium phosphate buffered water at pH 7.4. Kₐ=5.32×10⁵ M⁻¹.

FIG. 53 shows isothermal titration calorimetry (ITC) curve obtained through direct binding studies. A solution of 1 (30.0 μM) in the cell was titrated with 14 (300.0 μM) in the syringe at 298.0 K in 20 mM sodium phosphate buffered water at pH 7.4. Kₐ=3.02×10⁶ M⁻¹.

FIG. 54 shows isothermal titration calorimetry (ITC) curve obtained through direct binding studies. A solution of 1 (30.0 μM) in the cell was titrated with 15 (300.0 μM) in the syringe at 298.0 K in 20 mM sodium phosphate buffered water at pH 7.4. Kₐ=3.64×10⁶ M⁻¹.

FIG. 55 shows isothermal titration calorimetry (ITC) curve obtained through direct binding studies. A solution of 1 (100.0 μM) in the cell was titrated with 17 (1.00 mM) in the syringe at 298.0 K in 20 mM sodium phosphate buffered water at pH 7.4. Kₐ=7.69×10⁵ M⁻¹.

FIG. 56 shows isothermal titration calorimetry (ITC) curve obtained through direct binding studies. A solution of 1 (100.0 μM) in the cell was titrated with 16 (1.00 mM) in the syringe at 298.0 K in 20 mM sodium phosphate buffered water at pH 7.4. Kₐ=1.89×10⁵ M⁻¹.

FIG. 57 shows isothermal titration calorimetry (ITC) curve obtained through direct binding studies. A solution of 1 (30.0 μM) in the cell was titrated with 18 (300.00 μM) in the syringe at 298.0 K in 20 mM sodium phosphate buffered water at pH 7.4. Kₐ=4.85×10⁶ M⁻¹.

FIG. 58 shows top view and side view of X-ray single crystal structure of host 1 and guest 6d (capped sticks of guest 6d and space fill model of host 1).

FIG. 59 shows top view and side view of X-ray single crystal structure of host 1 and guest 6a (capped sticks of guest 6a and space fill model of host 1).

FIG. 60 shows HepG2 cell death assay performed after incubation with M0. Adenylate kinase (AK) assay was performed using supernatant from cells seeded for MTS assay (UT=untreated). This figure is the average and SEM values representative of two replicate experiments. Statistical analysis is one way ANOVA with Dunnett's multiple comparisons test. ****P<0.0001.

FIG. 61 shows HepG2 cell viability assay performed after incubation with M0. MTS assay was performed after the cells were incubated with container for 24 h (UT=Untreated). This figure is the average and SEM values representative of two replicate experiments. Statistical analysis is one-way ANOVA with Dunnett's multiple comparison test. ****P<0.0001.

FIG. 62 shows HEK293 cell death assay performed after incubation with M0. AK assay was performed using supernatant from cells seeded for MTS assay (UT=untreated). This figure is the average and SEM values representative of two replicate experiments. Statistical analysis is one way ANOVA with Dunnett's multiple comparisons test. ****P<0.0001.

FIG. 63 shows HEK293 cell viability assay performed after incubation with M0. MTS assay was performed after the cells were incubated with container for 24 h (UT=Untreated). This figure is the average and SEM values representative of two replicate experiments. Statistical analysis is one way ANOVA with Dunnett's multiple comparisons test. *P=0.01-0.05; ***P=0.0001-0.0005.

FIG. 64 shows M0 does not inhibit the hERG channel. The hERG assay was conducted using HEK-293 stably transfected with hERG cDNA in an automated QPatch HTX patch clamp study. Plot of mean hERG ion channel inhibition (%, n=3-4) versus log concentration for E-4031 (⋅) and M0 (∘).

FIG. 65 shows MTD study performed for M0. Female Swiss Webster mice (n=5 per group) were dosed via tail vein on days 0 and 2 (denoted by *) with different concentrations of M0 or 5% aqueous dextrose (D5W). The normalized average weight change per study group is indicated. Error bars represent SEM.

FIG. 66 shows ¹H NMR (400 MHz, DMSO) spectra recorded for compound 31.

FIG. 67 shows ¹H NMR (400 MHz, D₂O) spectra recorded for compound 32.

FIG. 68 shows ¹H NMR (400 MHz, DMSO) spectra recorded for compound 33.

FIG. 69 shows ¹H NMR (400 MHz, D₂O) spectra recorded for compound 34.

FIG. 70 shows ¹H NMR DMSO-d₆ spectra (400 MHz, DMSO-d₆, RT) recorded for compound 35.

FIG. 71 shows ¹³C NMR DMSO-d₆ spectra (100 MHz, DMSO-d₆, RT) recorded for compound 35.

FIG. 72 shows ¹H NMR D₂O spectra (400 MHz, D₂O, RT) recorded for 36.

FIG. 73 shows ¹³C NMR spectra (100 MHz, D₂O, RT) recorded for 36.

FIG. 74 shows ¹H NMR DMSO-d₆ spectra (400 MHz, DMSO-d₆, RT) recorded for compound 37.

FIG. 75 shows ¹³C NMR DMSO-d₆ spectra (100 MHz, DMSO-d₆, RT) recorded for compound 37.

FIG. 76 shows ¹H NMR D₂O spectra (400 MHz, D₂O, RT) recorded for 38.

FIG. 77 shows ¹³C NMR spectra (100 MHz, D₂O, RT) recorded for 38.

FIG. 78 shows ¹H NMR spectrum (400 MHz, DMSO-d₆, RT) recorded for compound 39.

FIG. 79 shows ¹H NMR spectrum (600 MHz, D₂O, RT) recorded for 40.

FIG. 80 shows ¹³C NMR spectrum (150 MHz, D₂O, RT) recorded for 40.

FIG. 81 shows ¹H NMR DMSO-d₆ spectra (400 MHz, DMSO-d₆, RT) recorded for compound 41

FIG. 82 shows ¹³C NMR DMSO-d₆ spectra (100 MHz, DMSO-d₆, RT) recorded for compound 41.

FIG. 83 shows ¹H NMR DMSO-d₆ spectra (400 MHz, DMSO-d₆, RT) recorded for compound 42.

FIG. 84 shows ¹³C NMR spectra (100 MHz, D₂O, RT) recorded for 42.

FIG. 85 shows in vivo reversal of methamphetamine-induced hyperlocomotion by Motor0. Average locomotion counts for male Swiss Webster mice (n=15; avg weight (g)±SD: 33.27±1.44) are plotted as a function of treatment. All mice underwent an initial habituation to determine baseline locomotion levels before treatment. Following this baseline measure, treatment order was counterbalanced across days, and mice only received one treatment per day. Over six consecutive days of testing mice each received a single treatment of 5% aqueous dextrose (D5W; 0.2 mL infused), Motor0 only (Motor0; 6 mM in D5W; 0.178 mL infused), methamphetamine only (4.24 mM Meth in D5W; 0.5 mg/kg; 0.022 mL infused), a premixed solution of Motor0 and methamphetamine (Premix; ˜11.6:1 Motor0:Meth; 0.2 mL infused), Motor0 followed by methamphetamine administered 30 s later (30s Blocking; 0.178 mL of 6 mM Motor0 in D5W, 0.022 mL of 4.24 mM Meth in D5W infused), and methamphetamine followed by Motor0 administered 30 s later (30 s Reversal; 0.022 mL of 4.24 mM Meth in D5W, 0.178 mL of 6 mM Motor0 in D5W infused). Bars represent average locomotion counts. Error bars represent the standard error of the mean (SEM). Dots represent counts for each mouse (n=15). Presented p-values are only for significant (p<0.05) Tukey-corrected post-hoc comparisons.

FIG. 86 shows in vivo reversal of methamphetamine-induced hyperlocomotion by Motor0 following 5-minute inter-injection interval. Average locomotion counts for male Swiss Webster mice (n=15; avg weight (g)±SD: 33.27±1.44) are plotted as a function of treatment. Mice receive either methamphetamine (4.24 mM Meth in D5W; 0.5 mg/kg; 0.022 mL infused) followed by D5W (0.178 mL infused) or Motor0 (6 mM in D5W; 0.178 mL infused) administered 5 minutes apart before being placed into the behavioral box. Bars represent average locomotion counts. Error bars represent the standard error of the mean (SEM). Dots represent counts for each mouse (n=15). Data analyzed using a paired t-test.

FIG. 87 shows the structure for M2.

FIG. 88 shows the chemical structures of the compounds used in the binding assays described in sample Example 3.

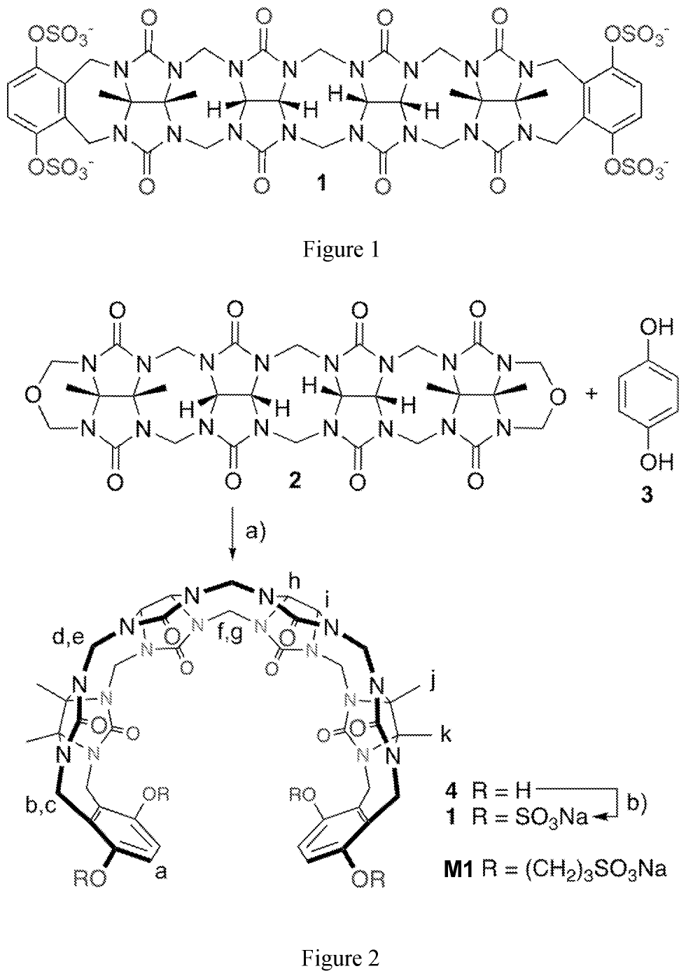

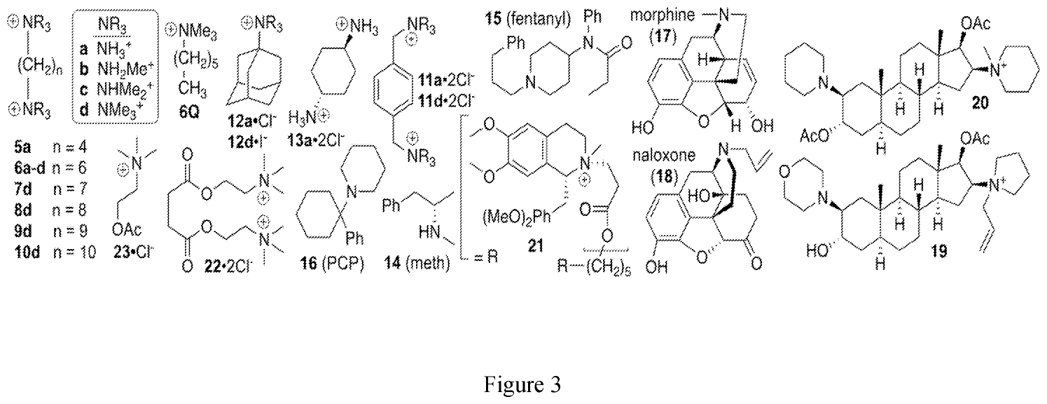

### DETAILED DESCRIPTION OF THE DISCLOSURE

Although claimed subject matter will be described in terms of certain embodiments and examples, other embodiments and examples, including embodiments and examples that do not provide all of the benefits and features set forth herein, are also within the scope of this disclosure. Various structural, logical, and process step changes may be made without departing from the scope of the disclosure.

All ranges provided herein include all values that fall within the ranges to the tenth decimal place, unless indicated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

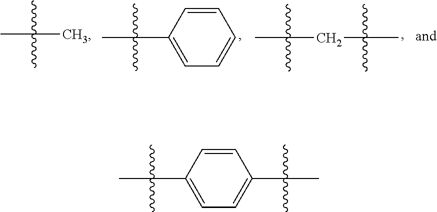

As used herein, unless otherwise indicated, the term “aryl group” refers to C₅ to C₁₈, including all integer numbers of carbons and ranges of numbers of carbons therebetween, aromatic or partially aromatic carbocyclic groups (e.g., C₁, C₂, C₃, C₄, C₅, C₆, C₇, C₈, C₉, C₁₀, C₁₁, C₁₂, C₁₃, C₁₄, C₁₅, C₁₆, C₁₇, and C₁₈). An aryl group may also be referred to as an aromatic group. The aryl groups can comprise polyaryl groups such as, for example, fused ring or biaryl groups. The aryl group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, thioether groups, thioester groups, and the like, and combinations thereof. A substituent may be or further comprise a sulfonate group or a sulfate group. Examples of aryl groups include, but are not limited to, phenyl groups, biaryl groups (e.g., biphenyl groups and the like), and fused ring groups (e.g., naphthyl groups, anthracene groups, pyrenyl groups, and the like), which may be unsubstituted or substituted.

As used herein, unless otherwise indicated, the term “heteroaryl group” refers to a C₁ to C₁₈ monocyclic, polycyclic, or bicyclic ring groups (e.g., aryl groups) comprising one or two aromatic rings containing at least one heteroatom (e.g., nitrogen, oxygen, sulfur, and the like) in the aromatic ring(s), including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, and C₁₈). The heteroaryl groups may be substituted or unsubstituted. Examples of heteroaryl groups include, but are not limited to, benzofuranyl groups, thienyl groups, furyl groups, pyridyl groups, pyrimidyl groups, oxazolyl groups, quinolyl groups, thiophenyl groups, isoquinolyl groups, indolyl groups, triazinyl groups, triazolyl groups, isothiazolyl groups, isoxazolyl groups, imidazolyl groups, benzothiazolyl groups, pyrazinyl groups, pyrimidinyl groups, thiazolyl groups, and thiadiazolyl groups, and the like. Examples of substituents include, but are not limited to, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof.

As used herein, unless otherwise indicated, the term “aliphatic” refers to branched or unbranched hydrocarbon groups that, optionally, contain one or more degree(s) of unsaturation. Degrees of unsaturation can arise from, but are not limited to, cyclic aliphatic groups. For example, the aliphatic groups/moieties are a C₁ to C₄₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). Aliphatic groups include, but are not limited to, alkyl groups, alkenyl groups, and alkynyl groups. The aliphatic group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, thioether groups, thioester groups, and the like, and combinations thereof.

As used herein, “carbocyclic group” refers to a cyclic compound having a ring or multiple rings in which all of the atoms forming the ring(s) are carbon atoms. The rings of the carbocyclic group can be aromatic or nonaromatic, and include compounds that are saturated and partially unsaturated, and fully unsaturated. Examples of such groups include benzene, naphthalene, 1,2-dihydronaphthalene, cyclohexane, cyclopentene, and the like. For example, the carbocyclic group can be a C₃ to C₂₀ carbocyclic group, including all integer numbers of carbons and ranges of numbers of carbons therebetween (e.g., C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). Aliphatic groups may be carbocyclic groups.

As used herein, “heterocyclic group” refers to a cyclic compound having a ring or multiple rings where at least one of the atoms forming the ring(s) is a heteroatom (e.g., oxygen, nitrogen, sulfur, etc.). The rings of the heterocyclic group can be aromatic or nonaromatic, and include compounds that are saturated, partially unsaturated, and fully unsaturated. Examples of such groups include imidazolidin-2-one, pyridine, quinoline, decahydroquinoline, tetrahydrofuran, pyrrolidine, pyrrolidone, and the like. For example, the heterocyclic group can be a C₁ to C₂₀ heterocyclic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀).

As used herein, “carbocyclic ring system” refers to a cyclic compound having a ring or multiple rings in which all of the atoms forming the ring(s) are carbon atoms. Examples of such groups include benzene, naphthalene, 1,2-dihydronaphthalene, cyclohexane, cyclopentene, and the like. The rings of the carbocyclic ring system or heterocyclic ring system can be aromatic or nonaromatic, and include compounds that are saturated, partially unsaturated, and fully unsaturated. For example, the carbocyclic ring system can be a C₃ to C₂₀ carbocyclic group, including all integer numbers of carbons and ranges of numbers of carbons therebetween (e.g., C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). In another example, the carbocyclic ring system can be a phenyl group or naphthyl group. The phenyl group or naphthyl group is attached to the compound via adjacent carbons of the phenyl group or naphthyl group.

As used herein, “heterocyclic ring system” refers to a cyclic compound having a ring or multiple rings in which at least one of the atoms forming the ring(s) is a heteroatom (e.g., oxygen, nitrogen, sulfur, etc.). The rings of the carbocyclic ring system or heterocyclic ring system can be aromatic or nonaromatic, and include compounds that are saturated, and fully unsaturated. Examples of the heterocyclic ring system include imidazolidin-2-one, pyridine, quinoline, decahydroquinoline, tetrahydrofuran, pyrrolidine, pyrrolidone, and the like. For example, the heterocyclic ring system can be a C₁ to C₂₀ heterocyclic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀).

As used herein, unless otherwise indicated, the term “alkyl group” refers to branched or unbranched saturated hydrocarbon groups. Examples of alkyl groups include, but are not limited to, methyl groups, ethyl groups, n- and isopropyl groups, n-, iso-, sec-, and tert-butyl groups, and the like. For example, the alkyl group can be a C₁ to C₁₂, including all integer numbers of carbons and ranges of numbers of carbons therebetween (e.g., C₁, C₂, C₃, C₄, C₅, C₆, C₇, C₈, C₉, C₁₀, C₁₁, and C₁₂). The alkyl group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups (—OR, where R is an alkyl group), carboxylate groups, carboxylic acid groups, ether groups, ester groups, amide groups, thioether groups, thioester groups, and the like, and combinations thereof.

The present disclosure provides acyclic sulfated cucurbit[n]uril with sulfate substituent(s). Also described herein are compositions comprising acyclic sulfated cucurbit[n]uril with sulfate substituent(s), methods of making acyclic sulfated cucurbit[n]uril with sulfate substituent(s), and methods of using acyclic sulfated cucurbit[n]uril with sulfate substituent(s).

In an aspect, the present disclosure provides compounds. The compounds are acyclic sulfated cucurbit[n]urils having one or more sulfate substituents. The compounds comprise a plurality of linked glycoluril groups.

In various examples, a compound has the following structure:

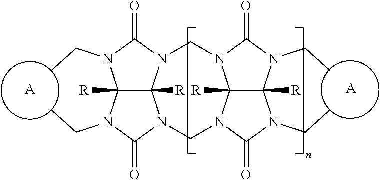

where each R is independently a hydrogen, a C₁ to C₂₀ alkyl group, a C₃ to C₂₀ carbocyclic group, a C₁ to C₂₀ heterocyclic group, a carboxylic acid group, a ester group, an amide group, a hydroxy, or an ether group. Optionally, adjacent R groups form a C₃ to C₂₀ carbocyclic ring or heterocyclic ring.

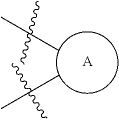

(or simply “A groups”)
is independently a C₅ to C₂₀ carbocyclic ring system or C₂ to C₂₀ heterocyclic ring system, where the ring system comprises one or more rings. At least one ring system has at least one ionizable group (e.g., solubilizing group) chosen from —OS(O)₂O⁻M⁺ and —OS(O)₂OH, where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and tris(hydroxymethyl)aminomethane (TRIS), and n is 0 to 6 (e.g., 1, 2, 3, 4, 5, 6). The compound may be a stereoisomer or mixture thereof, a salt, a partial salt, a hydrate, a polymorph or a mixture thereof of the above structure. In various embodiments, n is 3. In various embodiments, each R is independently hydrogen or methyl. Without intending to be bound by any particular theory, it is considered the ionizable group increases the solubility and/or binding affinity of the compound.

In various examples, M⁺is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and tris(hydroxymethyl)aminomethane (TRIS). In certain embodiments, M⁺ is Na⁺, K⁺, or H₄N⁺. In certain embodiments, M⁺ is Na⁺.

Various A groups may be used. For example, each A group is independently a C₅ to C₂₀ carbocyclic ring. Examples of A groups include, but are not limited to,

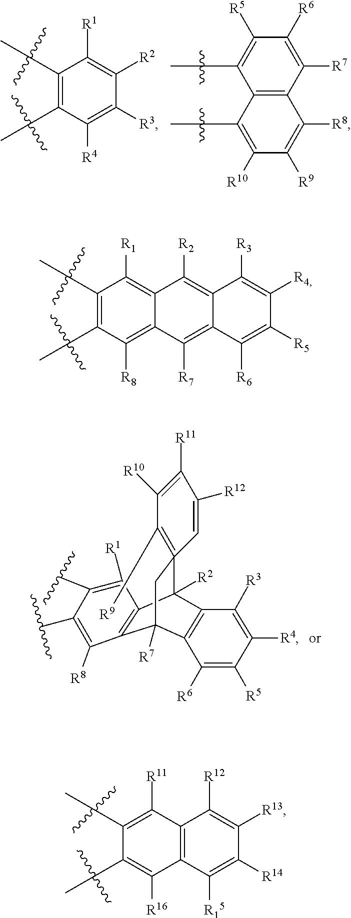

where at each occurrence of an A group, R¹ to R¹⁶ is independently chosen from hydrogen, —OS(O)₂O⁻M⁺ (wherein M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and trishydroxymethyl aminomethane (TRIS)), and —OS(O)₂OH, non-sulfate anionic groups (such as, for example, sulfonate (and corresponding acid) groups (e.g., —O(CH₂)ₘS(O)₂O⁻M⁺, or —O(CH₂)ₘS(O)₂OH, wherein m is 1 to 8, —C₆H₅S(O)₂OH, and the like and such groups where the terminal O is removed), carboxylate (and corresponding acid) groups (e.g., —O(CH₂)ₙC(O)O⁻ M⁺, —O(CH₂)ₘC(O)OH, wherein m is 1 to 8, and the like, such as for example, —OCH₂CO₂⁻M⁺, —OCH₂CO₂H groups and the like and such groups where the terminal O is removed), phosphonate (and corresponding acid) groups (e.g., —O(CH₂)ₘP(O)(OH)₂⁻M⁺ or —O(CH₂)ₘP(O)(OH)₂, wherein m is 1 to 8, and the like, such as for example, —O(CH₂)₂P(O)(OH)₂ and the like and such groups where the terminal O is removed), phosphate groups —OP(O)(OH)₂, and the like), substituted or unsubstituted aryl groups, substituted or unsubstituted heteroaryl groups, substituted or unsubstituted aliphatic groups (e.g., alkyl groups, alkenyl groups, alkynyl groups), O-alkyl groups (comprising an alkyl group), and azide groups, where at least one of R¹ to R¹⁶ is —OS(O)₂O⁻M⁺ or —OS(O)₂OH, and wherein M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and trishydroxymethyl aminomethane (TRIS).

In various embodiments, all the A groups are the same. In various other embodiments, all the A groups are

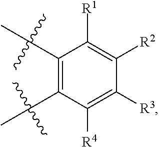

where R¹ and R⁴ are OS(O)₂O⁻M⁺ and R² and R³ are hydrogen.

In various embodiments, a compound of the present disclosure has the following structure:

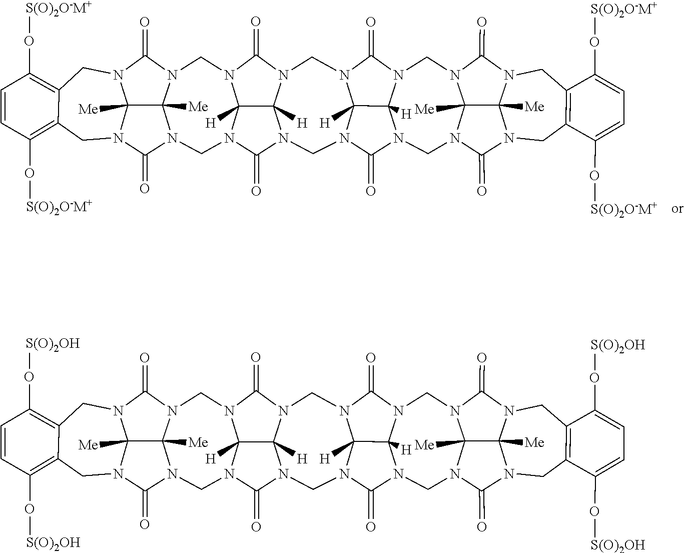

or a stereoisomer or mixtures thereof, a salt, a partial salt, a hydrate, a polymorph or a mixture thereof. In various examples, M⁺ is Na⁺, K⁺, H₄N⁺, Et₃NH⁺, Me₄N⁺, (HOCH₂CH₂)₃NH⁺, or a combination thereof. In various examples, M⁺ is Na⁺.

In an aspect, the present disclosure provides compositions comprising one or more compound(s). Non-limiting examples of compositions are described herein.

A composition may comprise one or more compound(s) and one or more pharmaceutical agent(s). In various examples, a pharmaceutical agent comprises one or more positively charged nitrogen atom(s) (e.g., ammonium ions, primary ammonium ions, secondary ammonium ions, tertiary ammonium ions, quaternary ammonium ions, or a combination thereof, where the non-hydrogen group(s) on the ammonium are chosen from aliphatic groups, alkyl groups, aryl groups, and combinations thereof).

A composition may comprise one or more compound (s), one or more pharmaceutical carrier(s), and, optionally, one or more pharmaceutical agent(s). The compositions described herein can be with one or more pharmaceutically acceptable carrier(s). Suitable pharmaceutically acceptable carriers are known in the art. Some non-limiting examples of pharmaceutically acceptable carriers can be found in: Remington: The Science and Practice of Pharmacy (2005) 21st Edition, Philadelphia, Pa. Lippincott Williams & Wilkins. In various examples, the pharmaceutical carrier is pure water or a buffer, such as PBS buffer or the like.

Compositions comprising one or more compound (s) combined with one or more pharmaceutical agent(s), which may form guest-host complexes, can be prepared at any point prior to use of the composition using any suitable technique. The compound-pharmaceutical agent complexes can be formed, for example, by mixing the compound and the pharmaceutical agent in a suitable solvent. It is desirable that the compound and pharmaceutical agent be soluble in the solvent such that the compound and agent form a non-covalent complex. Any suitable solvent can be used. In certain examples, the solvent is an aqueous solution, which includes, but is not necessarily limited to, water and various buffers (e.g., PBS buffer and the like). Non-aqueous solvents could also be used (e.g., MeOH, EtOH, DMSO, and other organic solvents, and combinations thereof), and then removed and the compositions if desired can be re-dissolved in an aqueous solution for administration. In general, a solution of a compound(s) can be provided at a known concentration, examples of which include but are not limited to from 0.1 to 90 mM, inclusive and including all integers to the tenth decimal place there between, and a pharmaceutical agent for which enhanced solubility is desired is added to the solution. The agent(s) can be provided, for example, in a solid form. The combination can be shaken or stirred for a period of time and the amount of pharmaceutical agent that is dissolved is monitored. If all added agent goes into solution, more agent can be added until some detectable portion of it remains undissolved (e.g., a solid). The soluble compound-agent complex can then be isolated and analyzed by any suitable technique, such as by recovering a centrifuged portion and analyzing it by NMR, to determine the concentration of pharmaceutical agent in solution. In various examples, a compound is provided in a composition comprising the drug at a ratio of at least 1 to 1 as pertains to the compound-agent stoichiometry (e.g., compound to drug ratio). In various examples, the compound (e.g., acyclic sulfated cucurbit[n]uril) to drug ratio is 100:1 to 1:5, including all ratio values and ranges therebetween (e.g., 100:1, 5:1, 1:2, 1:3, 1:4, or 1:5).

Compositions may be prepared at a patient's bedside or by a pharmaceutical manufacture. In the latter case, the compositions can be provided in any suitable container, such as, for example, a sealed sterile vial, ampoule, or the like, and may be further packaged (the combination of which may be referred to as a kit) to include instruction documents for use by a pharmacist, physician, other health care provider, or the like. The compositions can be provided as a liquid, or as a lyophilized or powder form that can be reconstituted if necessary when ready for use. In particular, the compositions can be provided in combination with any suitable delivery form or vehicle, examples of which include, but are not limited to, liquids, caplets, capsules, tablets, inhalants or aerosol, and the like. The delivery devices may comprise components that facilitate release of the pharmaceutical agents over certain time periods and/or intervals, and can include compositions that enhance delivery of the pharmaceuticals, such as nanoparticle, microsphere or liposome formulations, a variety of which are known in the art and are commercially available. Further, each composition described herein can comprise one or more pharmaceutical agent(s).

Compositions of the present disclosure may comprise more than one pharmaceutical agent. Likewise, the compositions can comprise distinct host-guest complexes. For example, a first composition comprising one or more acyclic sulfated cucurbit[n]urils and a first pharmaceutical agent can be separately prepared from a composition which comprises the same compound and a second pharmaceutical agent, and such preparations can be mixed to provide a two-pronged (or more) approach to achieving the desired prophylaxis or therapy in an individual. Further, compositions can be prepared using mixed preparations of any of the acyclic sulfated cucurbit[n]uril compounds disclosed herein.

A solid substrate may comprise one or more acyclic sulfated cucurbit[n]uril(s) disposed on (e.g., chemically bonded to) at least a portion of a surface of the substrate. At least a portion or all of the acyclic sulfated cucurbit[n]uril(s) may be chemically bonded to at least a portion of a surface by covalent bonds, non-covalent bonds, or a combination thereof. Methods of conjugating acyclic sulfated cucurbit[n]uril(s) to solid surfaces are known in the art. In various examples, acyclic sulfated cucurbit[n]uril(s) are conjugated to a surface by covalent bond- and/or non-covalent bond forming reactions including, but not limited to, amide bond formation, azide alkyne cycloaddition, gold thiol interactions, silicon alcohol condensations, and the like, and combinations thereof.

A solid substrate may comprise (or be) various materials. In various non-limiting examples, a solid substrate comprises or is silica (such as, for example, silica particles), polymer beads, polymer resins (such as, for example, polystyrene, poly NIPAM, polyacrylic acid), metal nanoparticles (e.g., gold nanoparticles, silver nanoparticles, magnetic nanoparticles), a metal (such as, for example, gold and the like), or the like, or a combination thereof.

In an aspect, the present disclosure provides uses of acyclic sulfated cucurbit[n]urils. Non-limiting examples of uses of acyclic sulfated cucurbit[n]urils are provided herein, for example, non-limiting examples of uses of acyclic sulfated cucurbit[n]urils are described in the Statements and Examples.

Acyclic sulfated cucurbit[n]urils can be used to sequester various materials, which may be chemical compounds. In various non-limiting examples, one or more acyclic sulfated cucurbit[n]urils(s) is/are used to sequester one or more neuromuscular blocking agent(s) (such as, for example, rocuronium, tubocurarine, atracurium, (cis)atracurium besylate, mivacurium, gallamine, pancuronium, vecuronium, and rapacuronium, and the like); one or more anesthesia agent(s) (such as, for example, N-methyl D-aspartate (NMDA) receptor antagonists (e.g., ketamine and the like), short-acting anesthetic agents (e.g., etomidate and the like), and the like); one or more pharmaceutical agent(s) (such as, for example, a drug (e.g., anticoagulants, such as, for example, hexadimethrine and the like), drugs of abuse (e.g., methamphetamine, cocaine, fentanyl, carfentanil, PCP, MDMA, heroin, and the like), and the like); one or more pesticide(s) (such as, for example, paraquat, diquat, organochlorines (e.g., DDT, aldrin, and the like), neonicotinoids (e.g., permethrin and the like), organophosphates (e.g., malathion, glyphosate, and the like), pyrethroids, triazines (e.g., atrazine and the like), and the like); one or more dyestuff(s) (such as, for example, methylene blue, nile red, crystal violet, thioflavin T, thiazole orange, proflavin, acridine orange, methylene violet, azure A, neutral red, cyanines, Direct orange 26, disperse dyes (e.g., disperse yellow 3, disperse blue 27, and the like), coumarins, congo red, and the like); one or more malodorous compound(s) (such as, for example, low molecular weight thiols (e.g., C₁-C₄ thiols), low molecular weight amines (e.g., triethylamine, putrescine, cadaverine, and the like), and the like); or one or more chemical warfare agent(s) (such as for example, nitrogen and sulfur mustards (e.g., bis(2-chloroethyl)ethylamine, bis(2-chloroethyl)methylamine, tris(2-chloroethyl)amine, bis(2-chloroethyl) sulfide, bis(2-chloroethylthioethyl) ether, and the like), nerve agents (such as, for example, those from the G, GV, and V series of nerve agents (e.g., tabun, sarin, soman, cyclosarin, 2-(dimethylamino)ethyl N,N-dimethylphosphoramidofluoridate (GV), novichok agents, VE, VG, VM, VX, and the like), and the like); one or more hallucinogen(s) (e.g., ergolines, lysergic acid diethylamide (LSD), psilocybin, tryptamines, dimethyltryptamine (DMT), phenethylamines, mescaline, ayahuasca, dextromethorphan, and the like); one or more toxin(s) (e.g., dioxins, perfluoralkylsulfonates (PFAS), perfluorooctanoic acid (PFOA), decabromobiphenyl ether (DECA), heavy metals (e.g., mercury), muscarine, tyramine, strychnine, tetrodotoxin, saxitoxin and the like, cholesterol, deoxycholic acid, N-Methyl-4-phenyl-1,2,3,6-tetrahydropyridine, phenylalanine, tyrosine, arginine, histamine); one or more metabolite(s) (e.g., toxic metabolites, such as, for example, N-methyl-4-phenylpyridine, spermine, spermidine, N-nitroso compounds e.g., 4-(methylnitrosoamino)-1-(3-pyridyl)-1-butanone); or the like, or a combination thereof.

A material, which may be a chemical compound, may comprise one or more cationic group. In various examples, a material, which may be a chemical compound, comprises one or more positively charged nitrogen atom(s) (e.g., ammonium ions, primary ammonium ions, secondary ammonium ions, tertiary ammonium ions, quaternary ammonium ions, or a combination thereof, where the non-hydrogen group(s) on the ammonium are chosen from aliphatic groups, alkyl groups, aryl groups, and combinations thereof).

In various examples, a method for sequestering one or more neuromuscular blocking agent(s), one or more anesthesia agent(s), one or more pharmaceutical agent(s), one or more pesticide(s), one or more dyestuff(s), one or more malodorous compound(s), one or more chemical warfare agent(s), one or more hallucinogen(s), one or more toxin(s), one or more metabolite(s) or the like, or a combination thereof comprises contacting the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof with one or more acyclic sulfated cucurbit[n]uril(s) and/or one or more composition(s), where the neuromuscular blocking agent(s), the anesthesia agent(s), the pharmaceutical agent(s), the pesticide(s), the dyestuff(s), the malodorous compound(s), the chemical warfare agent(s), or a combination thereof are sequestered by the one or more acyclic sulfated cucurbit[n]uril(s) and/or one or more composition(s).

The neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof may be present in an aqueous sample, in a solid sample (such as, for example, a soil sample), in a gas sample, or the like. An aqueous sample may be derived (e.g., via extraction or other methods to isolate the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof from the solid sample). The aqueous sample may be a wastewater sample (e.g., a municipal wastewater sample, industrial wastewater sample, and the like), an industrial water sample (e.g., water used to make a commercial product, such as, for example, a reagent, a solvent, or the like), a municipal water sample, or the like.

A composition may comprise one or more pharmaceutically active agent(s). In various non-limiting examples, at least a portion (or all) of the one or more compound(s) have a pharmaceutically active agent(s) disposed in the cavity of the one or more compound(s). Without intending to be bound by any particular theory, it is considered that a complex (which may be referred to as a guest-host complex) is formed from (e.g., one or more interaction(s) between (e.g., one or more non-covalent interactions, such as, for example, one or more non-covalent bond(s), is formed between) the compound(s), which may be referred to as hosts, and the neuromuscular blocking agent(s), the anesthesia agent(s), the pharmaceutical agent(s), which may be pharmaceutical agent(s) with undesirable (e.g., low) water solubility, the pesticide(s), the dyestuff(s), the malodorous compound(s), the chemical warfare agent(s), the hallucinogen(s), the toxin(s), the metabolite(s), or a combination thereof, which may be referred to a guest or guests. A guest-host complex can therefore be considered to be an organized chemical entity resulting from the association of the pharmaceutical agent(s) (guest(s)) and the host held together, for example, by non-covalent intermolecular forces.

A composition can comprise various pharmaceutically active agents. Non-limiting examples of pharmaceutical agents include drugs. The pharmaceutically active agent(s) may have various aqueous solubility. A pharmaceutically active agent may have hydrophobic, hydrophilic, or amphiphilic character.

The complexes may be removed from the aqueous sample, the solid sample, the gas sample, or the like. In various examples, the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof are removed from the aqueous sample, the solid sample, the gas sample, or the like using a solid surface with one or more acyclic sulfated cucurbit[n]uril(s) disposed thereon.

Acyclic sulfated cucurbit[n]urils can be used to sequester various materials in an individual. In various non-limiting examples, the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof is present in an individual and the contacting comprises administration of the one or more compound(s) and/or one or more composition(s) to the individual.

Acyclic sulfated cucurbit[n]urils can be used to reverse drug-induced neuromuscular block and/or anesthesia and/or the effects of one or more drug(s), which may be drugs of abuse in an individual.

In various non-limiting examples, a method for reversing drug-induced neuromuscular block and/or anesthesia and/or the effects of one or more pharmaceutical agent(s) (e.g., one or more drug(s) of abuse) in or on an individual comprising administering to an individual in need of reversal of neuromuscular block and/or reversal of anesthesia and/or reversal of the effects of the one or more pharmaceutical agent(s) (e.g., one or more drug(s) of abuse), one or more acyclic sulfated cucurbit[n]urils, and/or one or more composition(s). The individual may be in need of reversal of drug-induced neuromuscular block. The individual may be in need of reversal of anesthesia. The individual may be in need of reversal of drug-induced neuromuscular block and anesthesia. The individual may be in need of reversal of the effects of one or more pharmaceutical agent(s), such as, for example, one or more drug(s), which may be drug(s) of abuse. The individual may have been exposed to the drug(s) of abuse (e.g., carfentanil and the like) in a terrorist attack.

The acyclic sulfated cucurbit[n]uril compounds may be used as containers to solubilize chemical compounds. Improvement of solubility for compounds in, for example, aqueous solutions, is desirable for studying drug compounds and for improvement of drug bioavailability for purposes such as, for example, therapeutic and/or prophylactic purposes. For example, the acyclic sulfated cucurbit[n]urils are be used to enhance the stability (e.g., decrease degradation, increase shelf life, and the like) of drugs in water, the solid state, or both.

In certain examples, the acyclic sulfated cucurbit[n]uril compounds can be used to rescue promising drug candidates, which have undesirable solubility and bioavailability, and thus alleviate the attrition in the drug development process for anti-cancer agents and agents intended to treat other diseases. The containers may be used for targeted delivery of drugs to particular cell types, such as, for example, tumor cells and the like, to increase the effectiveness of existing drugs, reduce their toxic side effect(s), or both.

In various examples, a composition comprises one or more acyclic sulfated cucurbit[n]uril(s) and one or more pharmaceutical agent(s). Such compositions may be provided as pharmaceutical preparations as described herein.

It is important to emphasize that the pharmaceutical agent(s) that can be included in compositions comprising one or more acyclic sulfated cucurbit[n]uril(s) and one or more pharmaceutical agent(s) is not particularly limited. In certain examples, the pharmaceutical agent(s) combined with one or more acyclic sulfated cucurbit[n]uril(s) is/are a pharmaceutical agent or agents that is/are poorly water-soluble. In certain other examples, the pharmaceutical agent(s) combined with one or more acyclic sulfated cucurbit[n]uril(s) is/are a pharmaceutical agent or agents that is/are water soluble.

Solubility of any particular pharmaceutical agent can be determined, if desired, using any of a variety of techniques that are well known to those skilled in the art. Solubility can be ascertained if desired at any pH, such as a physiological pH, and/or at any desired temperature. Suitable temperatures include, but are not necessarily limited to, from 4° C. to 70° C., inclusive, and including all integer ° C. values therebetween.

In connection with poorly soluble or low solubility pharmaceutical agents suitable for use in the present disclosure, in various examples, such agents are considered to be those which have a solubility of less than 100 μM in water or an aqueous buffer.

In various other examples, poorly soluble pharmaceutical agents are considered to include compounds, which are Biopharmaceutics Classification System (BCS) class 2 or class 4 drugs. The BCS is well known to those skilled in the art and is based on the aqueous solubility of drugs reported in readily available reference literature, and for drugs that are administered orally it includes a correlation of human intestinal membrane permeability. (See, for example, Takagi et al., (2006) Molecular Pharmaceutics, Vol. 3, No. 6, pp. 631-643.) The skilled artisan will therefore readily be able to recognize a drug as a member of BCS class 2 or class 4 from published literature, or can test a drug with an unknown BCS or other solubility value to determine whether it has properties consistent with either of those classifications, or for otherwise being suitable for use in the present disclosure. In an example, solubility is determined according to the parameters set forth in this matrix:

| | Parts of solvent required for | Solubility Range |
|---|---|---|
| Solubility | 1 part of solute | (mg/mL) |
| very soluble | <1 | ≥1000 |
| freely soluble | from 1 to 10 | 100-1000 |
| soluble | from 10 to 30 | 33-100 |
| sparingly soluble | from 30 to 100 | 10-33 |
| slightly soluble | from 100 to 1000 | 1-10 |
| very slightly soluble | form 1000 to 10000 | 0.1-1 |
| practically insoluble | >10000 | <0.1 |

Thus, for the purposes of the present disclosure, a poorly soluble pharmaceutical agent that can be combined with one or more acyclic sulfated cucurbit[n]uril(s) can be any pharmaceutical agent that falls into the categories sparingly soluble, slightly soluble, very slightly soluble, and practically insoluble as set forth in the above matrix.

Again, it should be emphasized that other than being characterized as having low solubility in aqueous solution, the pharmaceutical agent with which one or more acyclic sulfated cucurbit[n]uril(s), which a compound can be combined is not limited. In this regard, at least one utility of the present disclosure is combination of one or more of a wide variety of distinct pharmaceutical agents with one or more acyclic sulfated cucurbit[n]uril(s), and as a consequence of combining these compounds with the pharmaceutical agent(s), solubility of the agent(s) is/are increased. In various examples, types of pharmaceutical agents suitable for solubilization include, but are not limited to, mitotic inhibitors (e.g., taxol, a mitotic inhibitor used in cancer chemotherapy, and the like); nitrogen mustard alkylating agents (e.g., Melphalan, trade name Alkeran used for chemotherapy, and the like); benzimidazoles (e.g., Albendazole, marketed as Albenza, Eskazole, Zentel and Andazol, for treatment of a variety of worm infestations, and the like); antagonists of the estrogen receptor in breast tissue which is used to treat breast cancers (e.g., Tamoxifen, which is an estrogen receptor antagonist when metabolized to its active form of hydroxytamoxifen, and the like); antihistamines (e.g., Cinnarizine, marketed as Stugeron and Stunarone for control of symptoms of motion sickness, and the like); thienopyridine class antiplatelet agents (e.g., Clopidogrel, marketed as Plavix for inhibiting blood clots in coronary artery disease and for other conditions, and the like); and antiarrhythmic agents (e.g., Amiodarone, used for treatment of tachyarrhythmias, and the like). Other pharmaceutical agents not expressly listed here are also included within the scope of the disclosure. Some examples of such agents include, but are not limited to, adjuvants for use in enhancing immunological responses, analgesic agents, detectably labeled agents used for diagnostic imaging, and the like. Combinations of any of these example pharmaceutical agents may be used. Acyclic sulfated cucurbit[n]urils may be combined with and improve solubility of pharmaceutical agents that are members of vastly different classes of compounds which are characterized by disparate chemical structures and biological activities.

Compositions of the present disclosure can be administered to any human or non-human animal in need of therapy or prophylaxis for one or more condition(s) for which the pharmaceutical agent is intended to provide a prophylactic of therapeutic benefit. Thus, the individual can be diagnosed with, suspected of having, or be at risk for developing any of a variety of conditions for which a reduction in severity would be desirable. Non-limiting examples of such conditions include cancer, including solid tumors, blood cancers (e.g., leukemia, lymphoma, myeloma, and the like). Specific examples of cancers include, but are not limited to, fibrosarcoma, myxosarcoma, liposarcoma, chondrosarcoma, osteogenic sarcoma, chordoma, angiosarcoma, endotheliosarcoma, lymphangiosarcoma, pseudomyxoma peritonei, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, head and neck cancer, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, thymoma, Waldenstrom's macroglobulinemia, heavy chain disease, and the like.

In addition to various malignancies, compounds of the present disclosure are also suitable for providing a benefit for cardiovascular related disorders, examples of which include, but are not limited to, angina, arrhythmia, atherosclerosis, cardiomyopathy, congestive heart failure, coronary artery disease, carotid artery disease, endocarditis, coronary thrombosis, myocardial infarction, hypertension, hypercholesterolemia/hyperlipidemia, mitral valve prolapse, peripheral artery disease, stroke, thrombosis, embolism, other forms of ischemic damage, and the like.

In addition, the compositions of the present disclosure can be used in connection with treating a variety of infectious diseases. It is expected that a variety of agents used to treat and/or inhibit infectious diseases caused by, for example, bacterial, protozoal, helminthic, fungal origins, viral origins, or the like can be aided by use of compositions of the present disclosure.

Various methods known to those skilled in the art can be used to introduce the compounds and/or compositions of the present disclosure to an individual. These methods include, but are not limited to, intravenous, intramuscular, intracranial, intrathecal, intradermal, subcutaneous, oral routes, and the like, and combinations thereof. The dose of the composition comprising a compound and a pharmaceutical agent will necessarily be dependent upon the needs of the individual to whom the composition is to be administered. These factors include, but are not necessarily limited to, the weight, age, sex, medical history, and nature and stage of the disease for which a therapeutic or prophylactic effect is desired. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using ordinary skill in the art, and given the benefit of the present disclosure.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like.

The steps of the method described in the various examples disclosed herein are sufficient to carry out the methods of the present disclosure. Thus, in an example, the method consists essentially of a combination of the steps of the methods disclosed herein. In another example, the method consists of such steps.

In an aspect, the present disclosure provides articles comprising compounds of the present disclosure.

The articles may be articles of manufacture. Non-limiting examples of articles include wipes impregnated with one or more compounds of the present disclosure. For example, such a wipe is used to decontaminate a surface from any material capable of being sequestered by a compound (e.g., acyclic sulfated cucurbit[n]uril of the present disclosure). For example, the wipe is used to decontaminate a surface that has or was previously exposed to a toxin, abused drug, or the like, or a combination thereof.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

The following Statements describe various embodiments of the present disclosure.

Statement 1. A compound having the following structure:

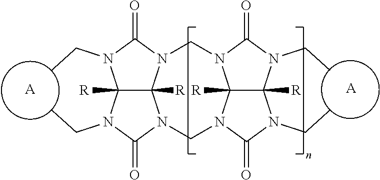

where each R is independently a hydrogen, a C₁ to C₂₀ alkyl group, a C₃ to C₂₀ carbocyclic group, a C₁ to C₂₀ heterocyclic group, a carboxylic acid group, a ester group, an amide group, a hydroxy, or an ether group; where, optionally, adjacent R groups form a C₃ to C₂₀ carbocyclic ring or heterocyclic ring; where each

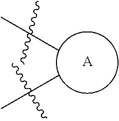

is independently a C₅ to C₂₀ carbocyclic ring system or C₂ to C₂₀ heterocyclic ring system, where the ring system comprises one or more rings; where at least one ring system has at least one ionizable group (e.g., solubilizing group) chosen from —OS(O)₂O⁻M⁺ and —OS(O)₂OH, where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and trishydroxymethyl aminomethane (TRIS), and where n is 0 to 6, or a stereoisomer or mixtures thereof, a salt, a partial salt, a hydrate, a polymorph or a mixture thereof.
Statement 2. A compound according to Statement 1, where each

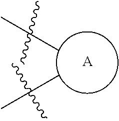

is independently a C₅ to C₂₀ carbocyclic ring having one of the following structures:

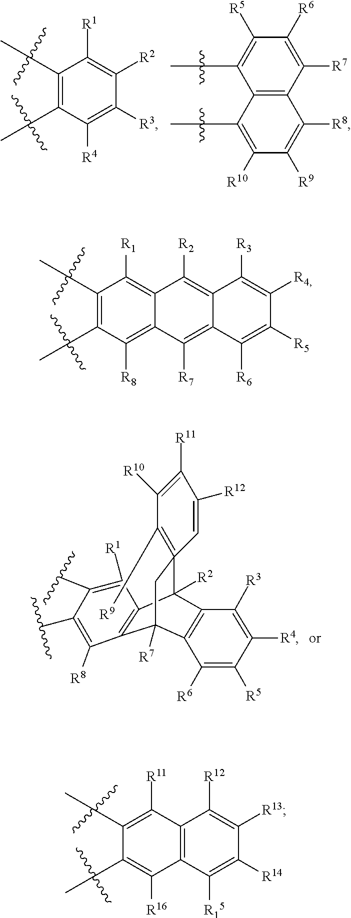

where at each occurrence of

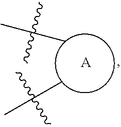

R¹ to R¹⁶ is independently chosen from hydrogen, —OS(O)₂O⁻M⁺ (where M⁺ is Na⁺, K⁺, Ca²⁺, Mg²⁺, Zn²⁺, H₄N⁺, Et₃NH⁺, Me₄N⁺, HOCH₂CH₂)₃NH⁺, or a cationic form of ethylenediamine, piperazine, and trishydroxymethyl aminomethane (TRIS)), and —OS(O)₂OH, non-sulfate anionic groups (such as, for example, sulfonate (and corresponding acid) groups (e.g., —O(CH₂)ₘS(O)₂O⁻M⁺, or —O(CH₂)ₘS(O)₂OH, where m is 1 to 8, —C₆H₅S(O)₂OH, and the like and such groups where the terminal O is removed), carboxylate (and corresponding acid) groups (e.g., —O(CH₂)ₙC(O)O⁻M⁺, —O(CH₂)ₘC(O)OH, where m is 1 to 8, and the like, such as for example, —OCH₂CO₂⁻M⁺, —OCH₂CO₂H groups and the like and such groups where the terminal O is removed), phosphonate (and corresponding acid) groups (e.g., —O(CH₂)ₘP(O)(OH)₂⁻M⁺ or —O(CH₂)ₘP(O)(OH)₂, where m is 1 to 8, and the like, such as for example, —O(CH₂)₂P(O)(OH)₂ and the like and such groups where the terminal O is removed), phosphate groups —OP(O)(OH)₂, and the like), substituted or unsubstituted aryl groups, substituted or unsubstituted heteroaryl groups, substituted or unsubstituted aliphatic groups (e.g., alkynyl groups, alkenyl groups, and alkyl groups), O-alkyl groups (comprising an alkyl group), and azide groups, where at least one of R¹ to R¹⁶ is —OS(O)₂O⁻M⁺ or —OS(O)₂OH, and where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and trishydroxymethyl aminomethane (TRIS).
Statement 3. A compound according to Statement 2, where the

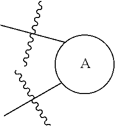

groups are the same.
Statement 4. A compound according to any one of the preceding Statements, where the

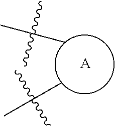

groups are

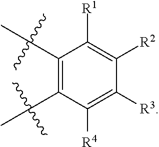

Statement 5. A compound according to any one of the preceding Statements, where R¹ and R⁴ is —OS(O)₂O⁻M⁺.
Statement 6. A compound according to any one of the preceding Statements, where n is 3.
Statement 7. A compound according to any one of the preceding Statements, where each R is independently hydrogen or methyl.
Statement 8. A compound according to any one of the preceding Statements, where R² and R³ are hydrogen.
Statement 9. A compound according to any one of the preceding Statements, where the compound has the following structure:

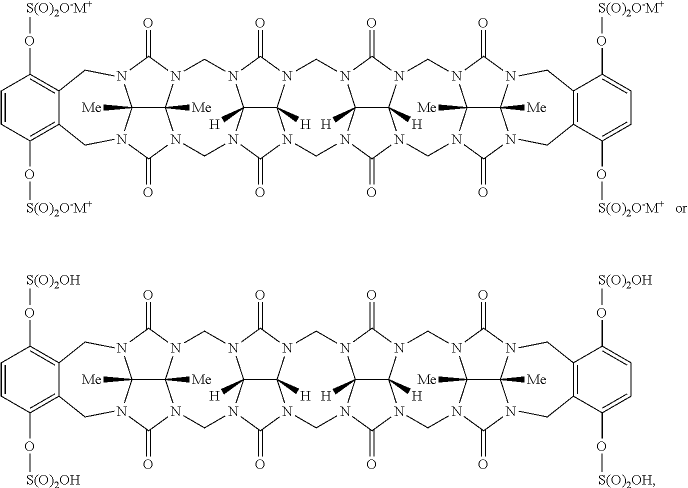

where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and tris(hydroxymethyl)aminomethane (TRIS), or a stereoisomer or mixtures thereof, a salt, a partial salt, a hydrate, a polymorph or a mixture thereof.
Statement 10. A composition comprising one or more compound(s) according to any one of the preceding Statements.
Statement 11. A composition according to Statement 10, further comprising a pharmaceutical carrier.
Statement 12. A composition according to Statements 10 or 11, further comprising a pharmaceutical agent.
Statement 13. A composition according to Statement 12, where the pharmaceutical agent is non-covalently complexed to the compound.
Statement 14. The composition according to Statements 12 or 13, where the pharmaceutical agent has a solubility of less than 100 μM in an aqueous solvent.
Statement 15. A composition according to Statements 10 or 11, where the one or more compound(s) is disposed (e.g., chemically bonded) to at least a portion of a solid substrate.
Statement 16. A composition according to Statement 15, where the solid substrate comprises (or is) silica (such as, for example, silica particles), polymer beads, polymer resins (such as, for example, polystyrene, poly NIPAM, polyacrylic acid, metal nanoparticles (e.g., gold nanoparticles, silver nanoparticles, magnetic nanoparticles), a metal (such as, for example, gold and the like), and the like.
Statement 17. A composition according to any one of Statements 15 and 16, where at least a portion (or all) of the one or more compound(s) have a pharmaceutically active agent(s) is non-covalently bound thereof (e.g., disposed in the cavity of the one or more compound(s)).
Statement 18. A method for sequestering: one or more neuromuscular blocking agent(s) (such as, for example, rocuronium, tubocurarine, atracurium, (cis)atracurium besylate, mivacurium, gallamine, pancuronium, vecuronium, and rapacuronium, and the like); one or more anesthesia agent(s) (such as, for example, N-methyl D-aspartate (NMDA) receptor antagonists (e.g., ketamine and the like), short-acting anesthetic agents (e.g., etomidate and the like), and the like); one or more pharmaceutical agent(s) (such as, for example, a drug (e.g., anticoagulants, such as, for example, hexadimethrine and the like), drugs of abuse (e.g., methamphetamine, cocaine, fentanyl, carfentanil, PCP, MDMA, heroin, and the like), and the like); one or more pesticide(s) (such as, for example, paraquat, diquat, organochlorines (e.g., DDT, aldrin, and the like), neonicotinoids (e.g., permethrin and the like), organophosphates (e.g., malathion, glyphosate, and the like), pyrethroids, triazines (e.g., atrazine and the like), and the like); one or more dyestuff(s) (such as, for example, methylene blue, nile red, crystal violet, thioflavin T, thiazole orange, proflavin, acridine orange, methylene violet, azure A, neutral red, cyanines, Direct orange 26, disperse dyes (e.g., disperse yellow 3, disperse blue 27, and the like), coumarins, congo red, and the like); one or more malodorous compound(s) (such as, for example, low molecular weight thiols (e.g., C₁-C₄ thiols), low molecular weight amines (e.g., triethylamine, putrescein, cadaverine, and the like), and the like); or one or more chemical warfare agent(s) (such as for example, nitrogen and sulfur mustards (e.g., bis(2-chloroethyl)ethylamine, bis(2-chloroethyl)methylamine, tris(2-chloroethyl)amine, bis(2-chloroethyl) sulfide, bis(2-chloroethylthioethyl) ether, and the like), nerve agents (such as, for example, those from the G, GV, and V series of nerve agents (e.g., tabun, sarin, soman, cyclosarin, 2-(dimethylamino)ethyl N,N-dimethylphosphoramidofluoridate (GV), novichok agents, VE, VG, VM, VX, and the like), and the like); one or more hallucinogen(s) (e.g., ergolines, lysergic acid diethylamide (LSD), psilocybin, tryptamines, dimethyltryptamine (DMT), phenethylamines, mescaline, ayahuasca, dextromethorphan, and the like); one or more toxin(s) (e.g., dioxins, perfluoralkylsulfonates (PFAS), perfluorooctanoic acid (PFOA), decabromobiphenyl ether (DECA), heavy metals (e.g., mercury), muscarine, tyramine, strychnine, tetrodotoxin, saxitoxin and the like, cholesterol, deoxycholic acid, N-Methyl-4-phenyl-1,2,3,6-tetrahydropyridine, phenylalanine, tyrosine, arginine, histamine); one or more metabolite(s) (e.g., toxic metabolites, such as, for example, N-methyl-4-phenylpyridine, spermine, spermidine, N-nitroso compounds e.g., 4-(methylnitrosoamino)-1-(3-pyridyl)-1-butanone); or the like, or a combination thereof are sequestered by the one or more compound(s) according to any one of Statements 1-10 and/or one or more composition(s) according to any one of Statements 11-17.
Statement 19. A method according to Statement 18, neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof is present in an aqueous sample, in a solid sample (such as, for example, a soil sample), in a gas sample, on a solid surface, or the like.
Statement 20. A method according to 19, where the aqueous sample is a wastewater sample (e.g., a municipal wastewater sample, industrial wastewater sample, and the like), an industrial water sample (e.g., water used to make a commercial product, such as, for example, a reagent, a solvent, or the like), a municipal water sample, or the like.
Statement 21. A method according to any one of Statements 18-20, where a complex is formed from (e.g., one or more interaction(s) between (e.g., one or more non-covalent bond(s) is formed between) the compound(s) and the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof.
Statement 22. A method of any one of Statements 18-21, where the complex is removed from the aqueous sample, the solid sample, the gas sample, or the like.
Statement 23. A method according to any one of Statements 18-22, where the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof is present in and/or on an individual and the contacting comprises administration of the one or more compound(s) and/or one or more composition(s) to the individual.
Statement 24. A method according to Statement 23, where the individual is a human or a non-human mammal.
Statement 25. A method for reversing drug-induced neuromuscular block and/or anesthesia and/or the effects of one or more drug(s) of abuse in an individual comprising administering to an individual in need of reversal of neuromuscular block and/or reversal of anesthesia and/or reversal of the effects of one or more pharmaceutical agent(s) (e.g., one or more drug(s) of abuse) one or more compound(s) according to any one of Statements 1-9 and/or one or more composition(s) according to any one of Statements 10-17.
Statement 26. A method according to Statement 25, where the individual is in need of reversal of drug-induced neuromuscular block.
Statement 27. A method according to Statement 25, where the individual is in need of reversal of anesthesia.
Statement 28. A method according to Statement 25, where the individual is in need of reversal of drug-induced neuromuscular block and anesthesia.
Statement 29. A method according to Statement 25, where the individual is in need of reversal of the effects of one or more pharmaceutical agent(s) chosen from one or more drug(s) of abuse, one or more pesticide(s), one or more chemical warfare agent(s), one or more nerve agent(s), one or more hallucinogen(s), one or more toxin(s), and/or one or more metabolite(s). In an example, the individual was exposed to the one or more drug(s) of abuse (e.g., carfentanil and the like), one or more pesticide(s), one or more chemical warfare agent(s), one or more nerve agent(s), one or more hallucinogen(s), one or more toxin(s), one or more metabolite(s) in a terrorist attack, and combinations thereof. In an example, the individual was exposed to the drug(s) of abuse (e.g., carfentanil and the like) in a terrorist attack.
Statement 30. A method according to any one of Statements 25-29, where the individual in need is a human.
Statement 31. A method according to any one of Statements 25-29, where the individual in need is a non-human mammal.
Statement 32. A method for prophylaxis and/or therapy of a condition in an individual comprising administering to an individual in need of the prophylaxis and/or the therapy a composition comprising a compound according to any one of Statements 1-9 or a composition according to any one of Statements 10-17, where subsequent to the administration the therapy and/or the prophylaxis of the condition in the individual occurs.
Statement 33. A method for prophylaxis and/or therapy of a condition in an individual comprising administering to an individual in need of the prophylaxis and/or the therapy (i) one or more compound(s) according to any one of Statements 1-9 or one or more composition(s) according to any one of Statements 10-17, and (ii) one or more pharmaceutical agent(s), where the compound(s) and the pharmaceutical agent(s) are present as complex (or a composition, which may be a pharmaceutical composition, comprising the complex(es)), where subsequent to the administration the therapy and/or the prophylaxis of the condition in the individual occurs.
Statement 34. A method according to Statement 33, where one or more of the pharmaceutical agent(s) has/have a solubility of less than 100 μM in an aqueous solvent.
Statement 35. A compound according to any one of Statements 1-9, the composition according to any one of Statements 10-17, or the method according to any one of Statements 18-34, where M⁺ is Na⁺, K⁺, H₄N⁺, Et₃NH⁺, Me₄N⁺, (HOCH₂CH₂)₃NH⁺.
Statement 36. A compound according to any one of Statements 1-9, the composition according to any one of Statements 10-17, or the method according to any one of Statements 18-34, where M⁺ is Na⁺.

The following examples are presented to illustrate the present disclosure. They are not intended to be limiting in any matter.

### Example 1

This example provides a description of compounds of the present disclosure and methods of making and using same.

It was reasoned that alkylene linkers result in the anionic sulfonate groups being positioned away from the ureidyl carbonyl portal of M1 (FIG. 2), for example, and thereby reduce the electrostatic driving force toward guest complexation. Accordingly, it was hypothesized that complete removal of the linker would position the negatively charged group closer to the cation binding site at the ureidyl carbonyl portal and thereby increase binding affinity toward cationic guests. Described herein is the synthesis of host 1 whose anionic sulfate groups are positioned at the ureidyl C═O portals to complement cationic guests.

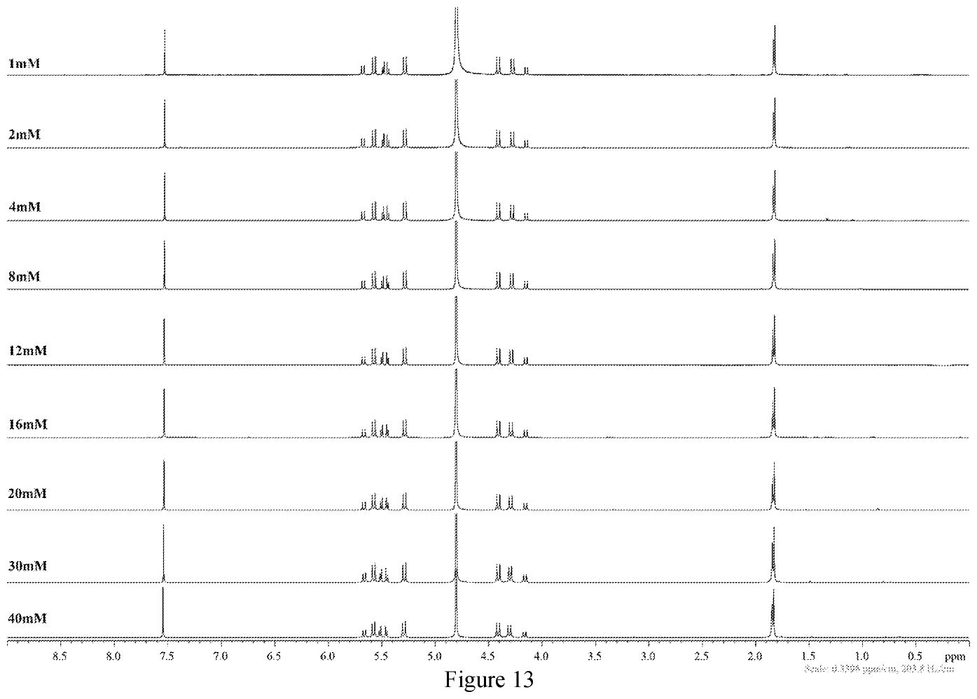

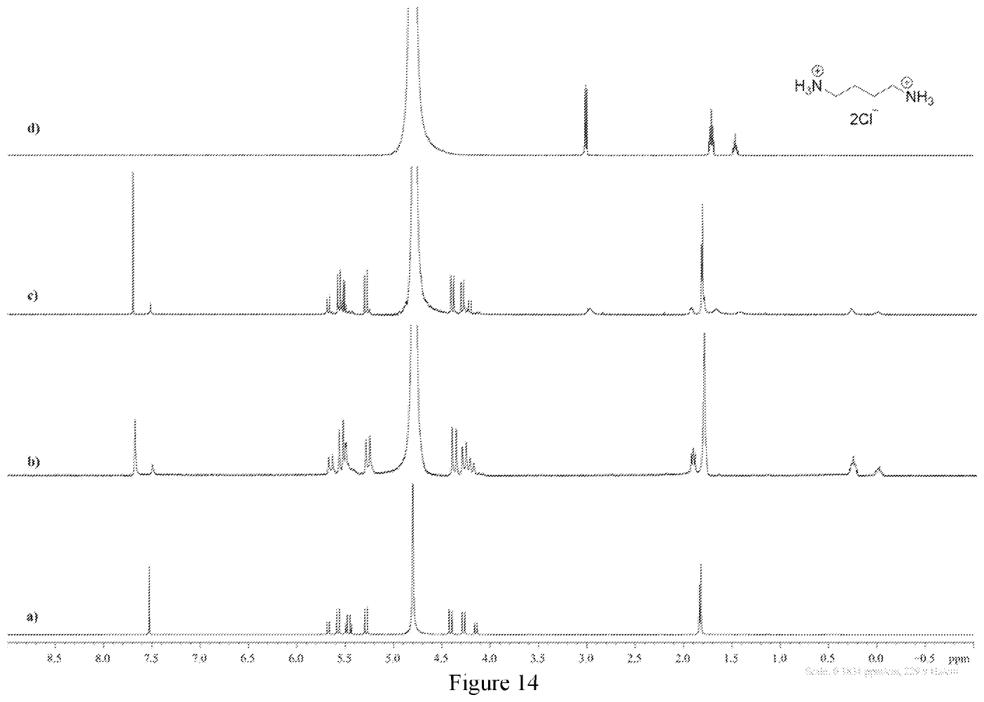

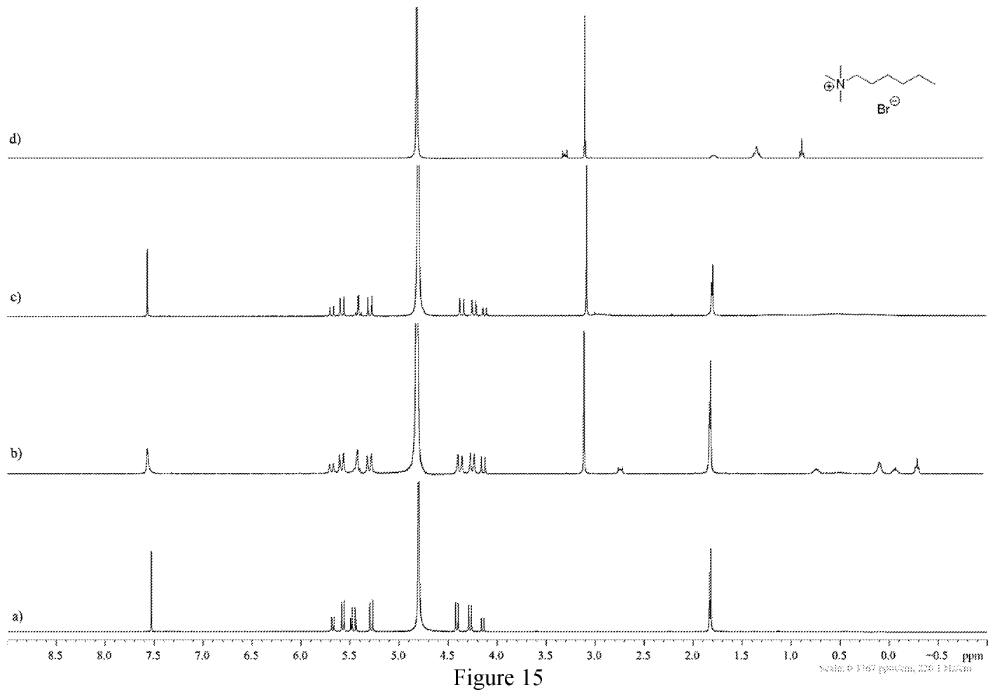

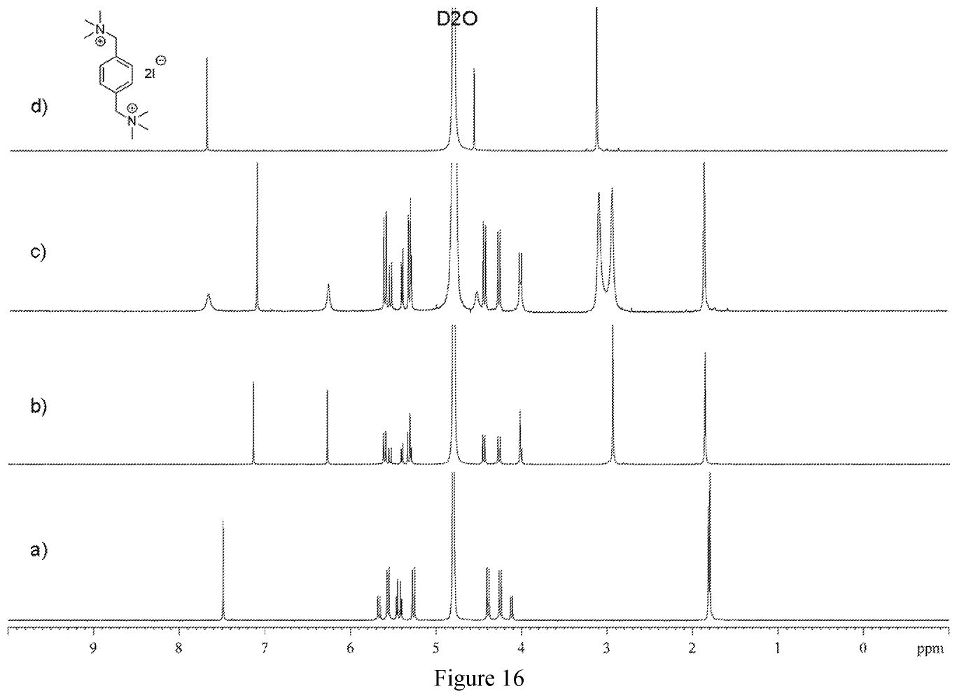

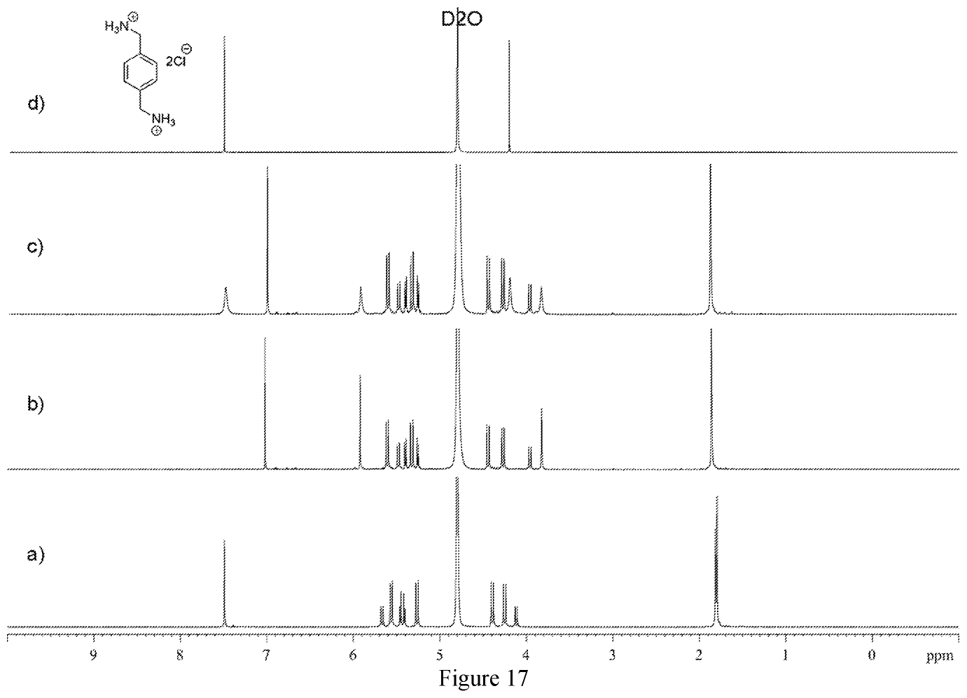

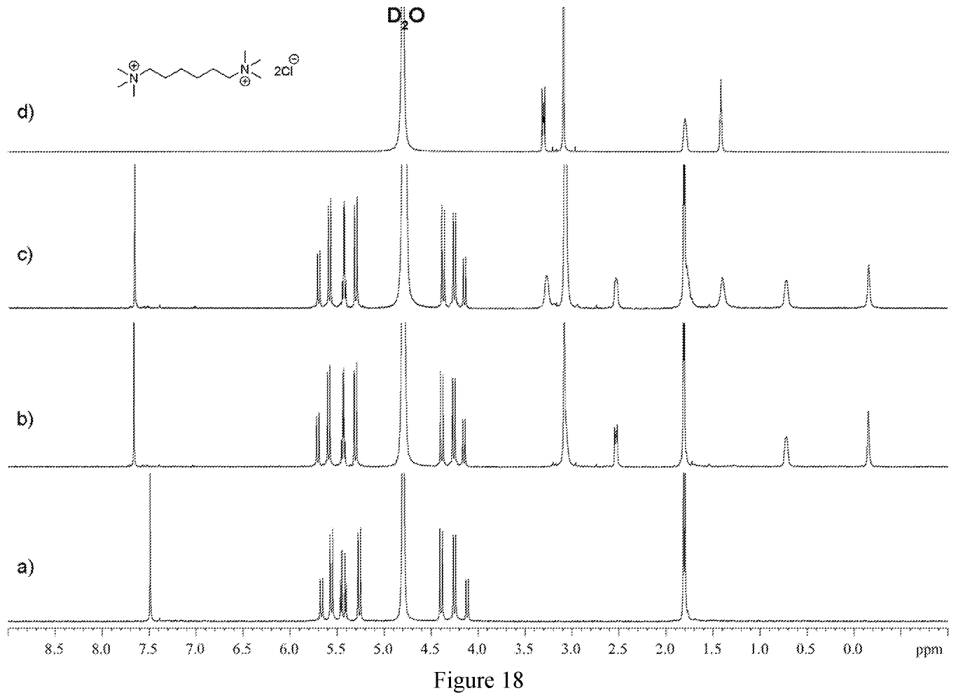

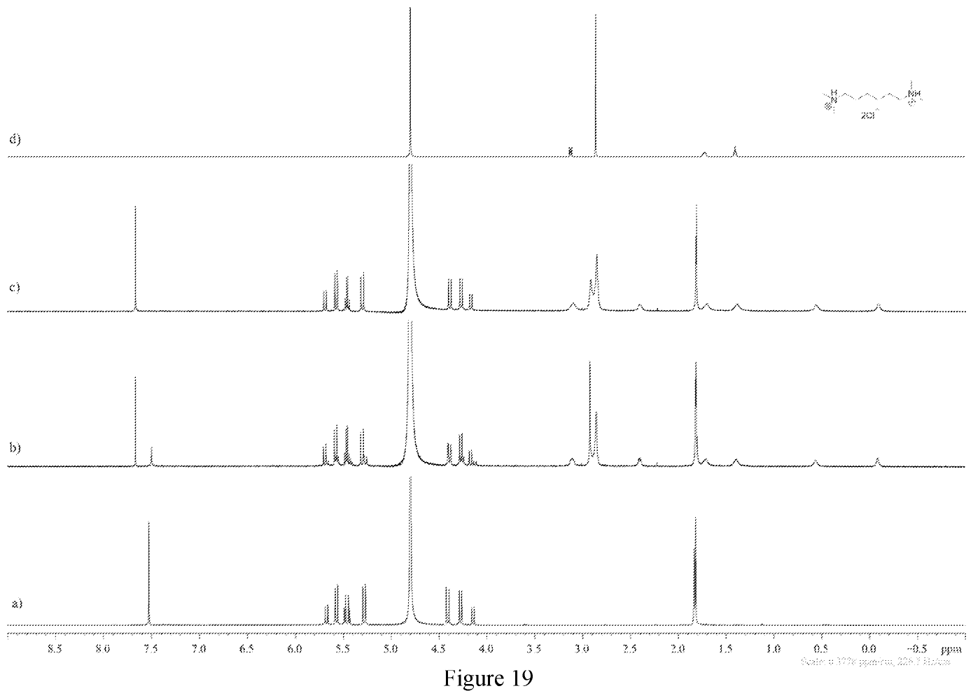

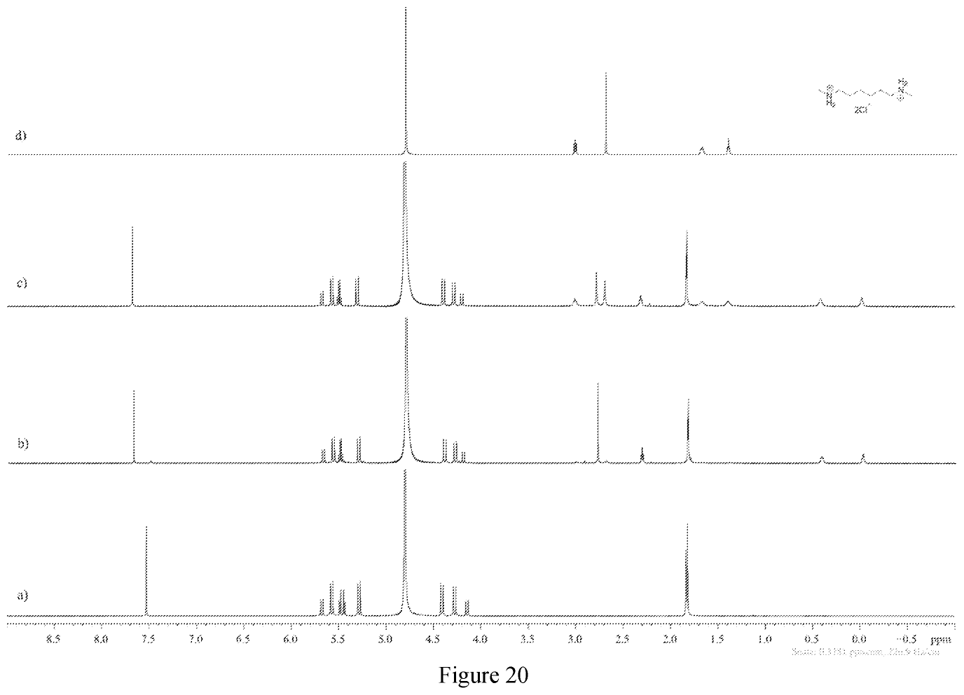

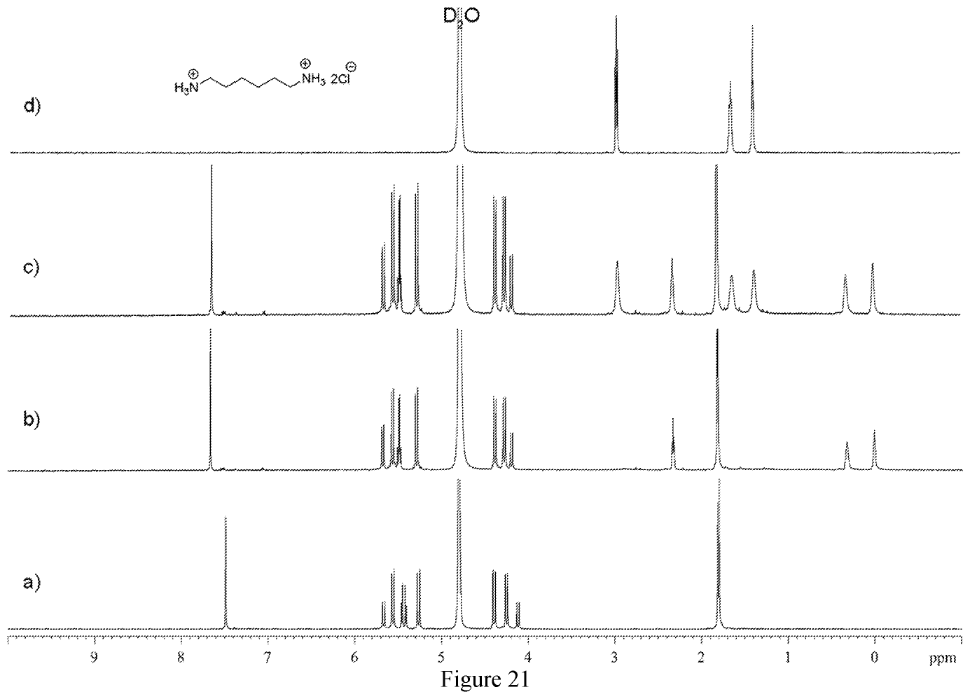

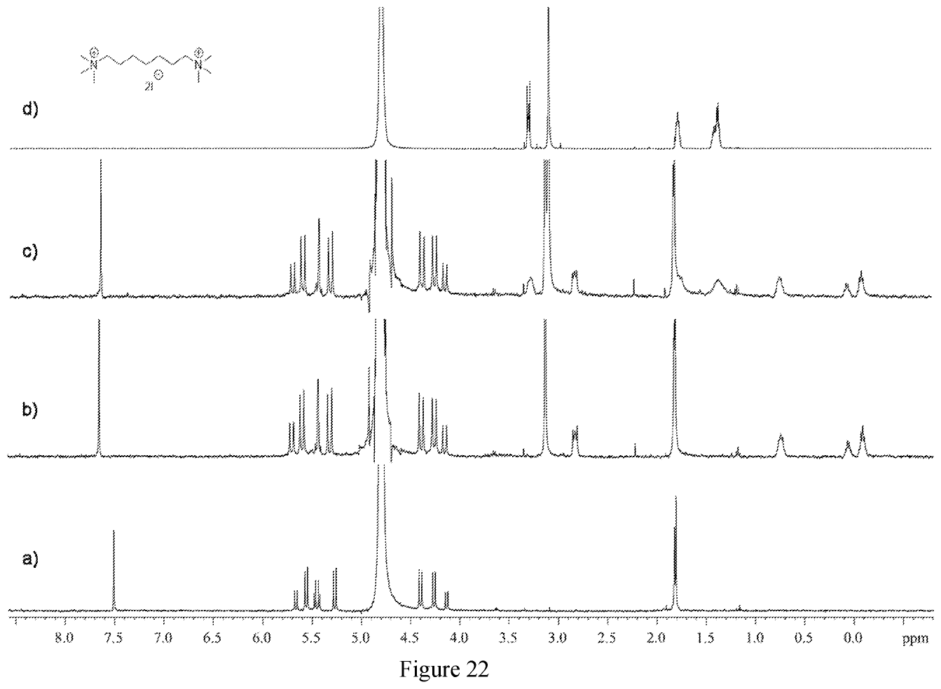

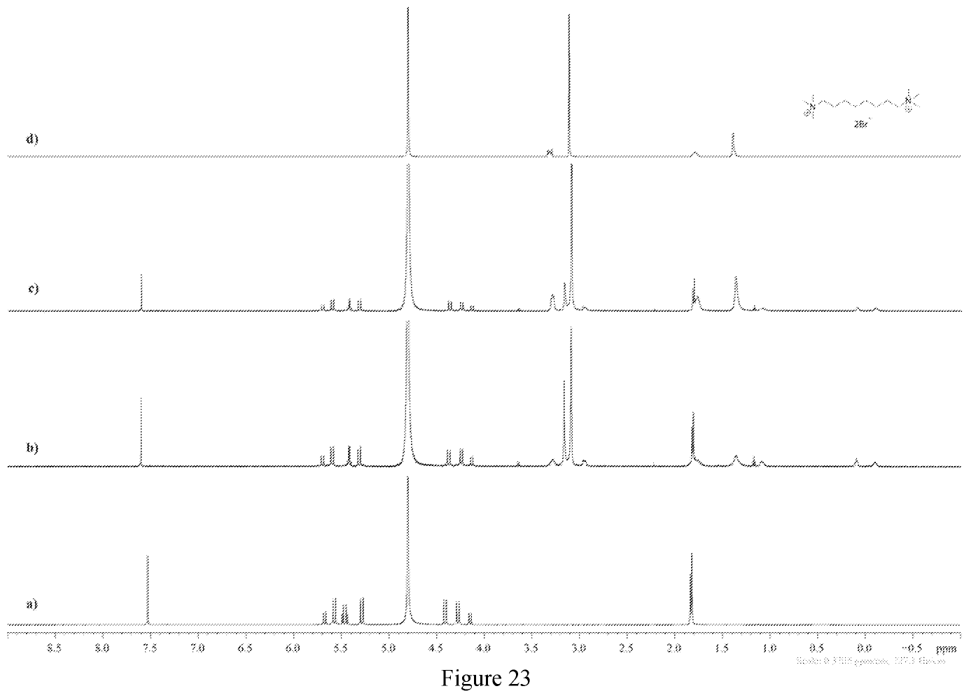

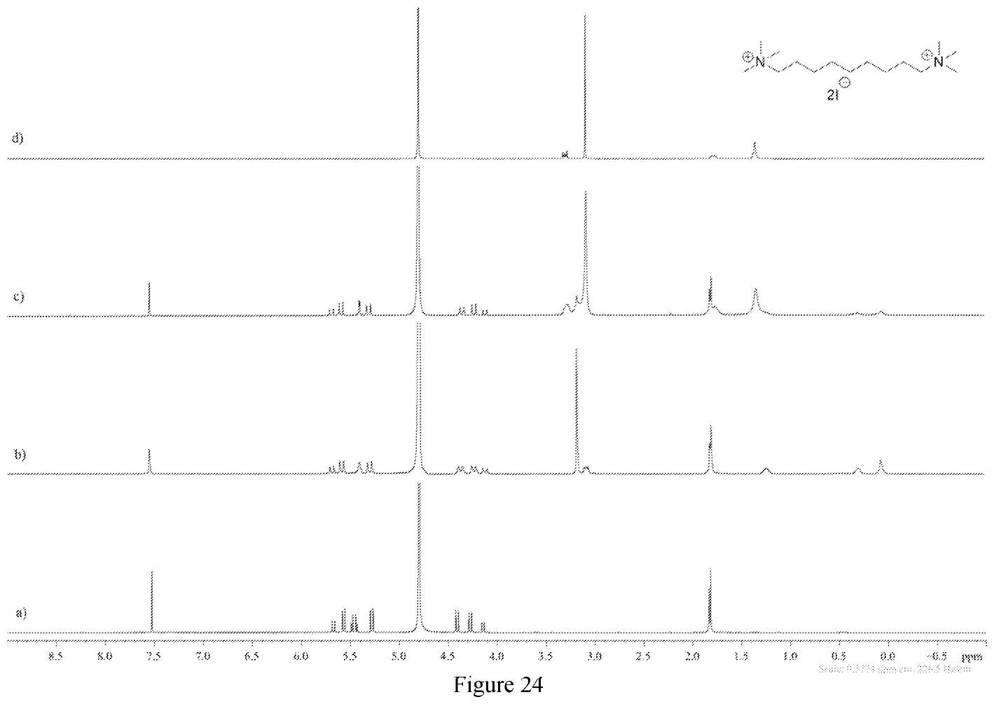

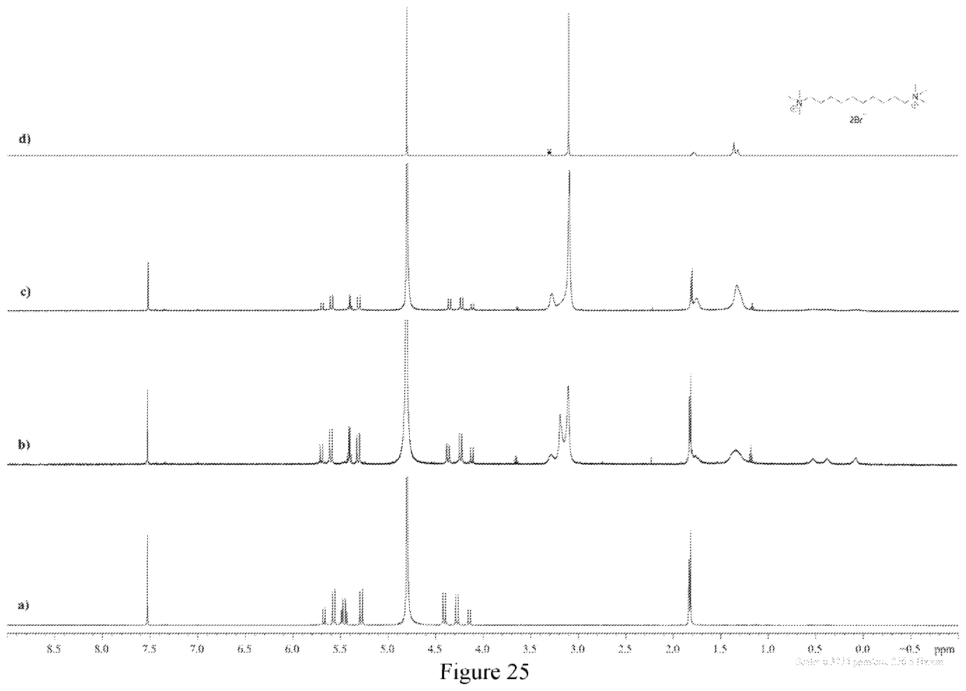

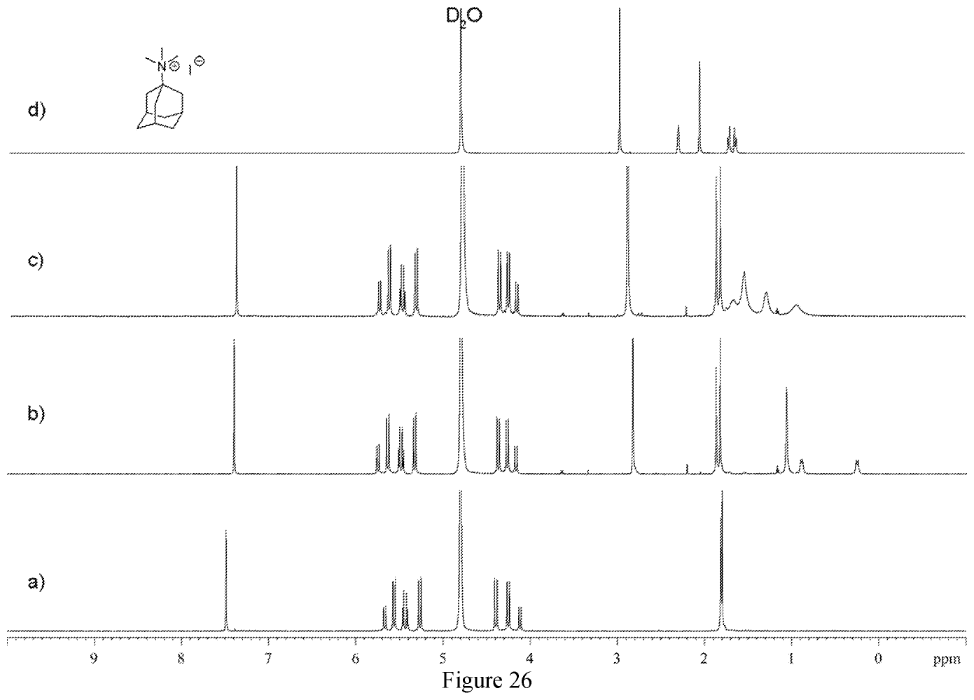

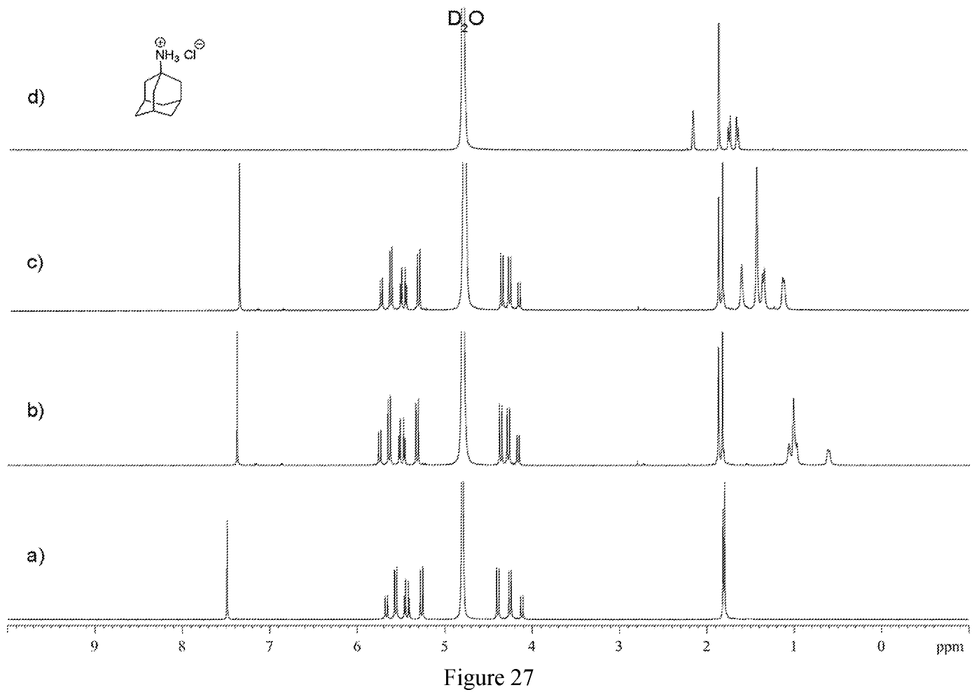

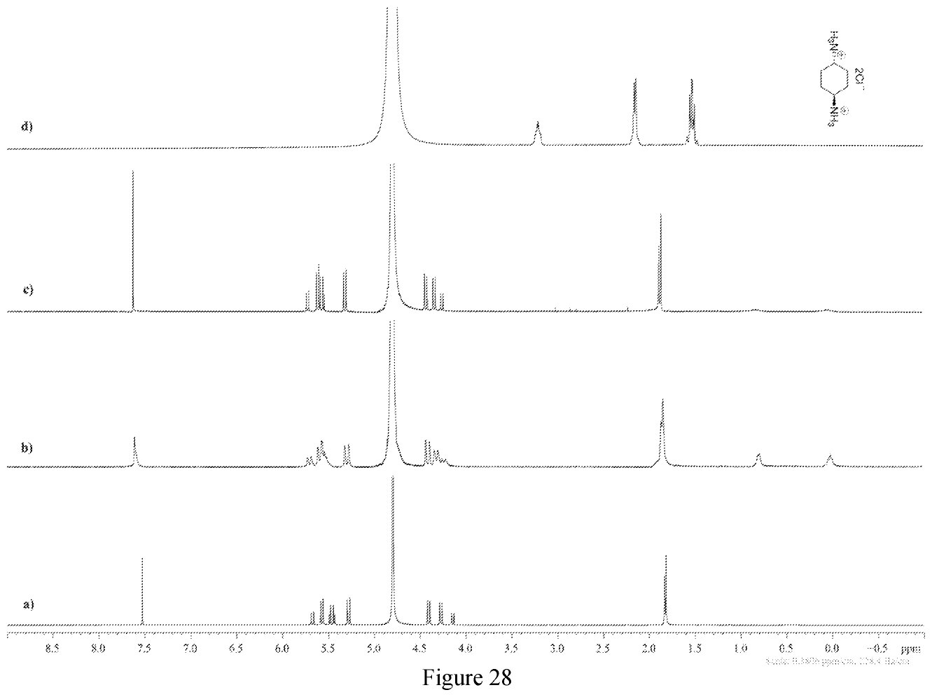

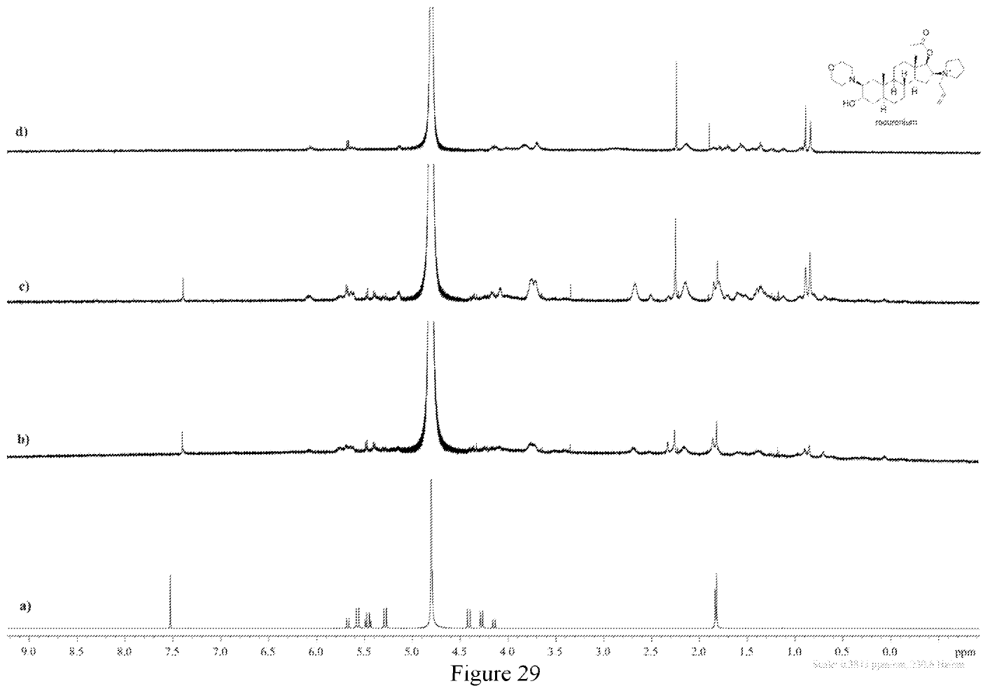

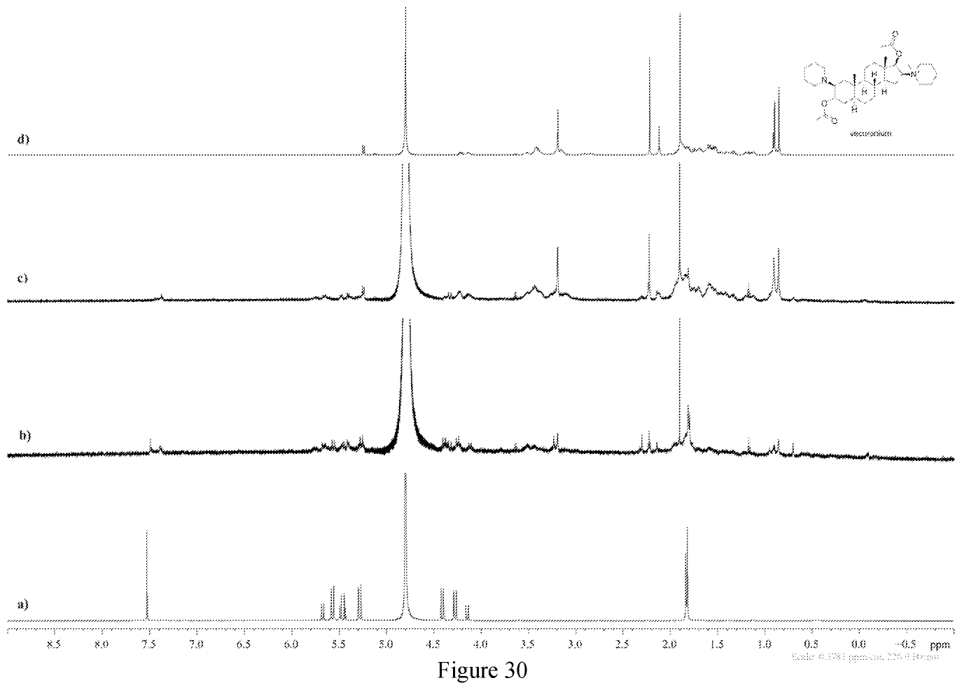

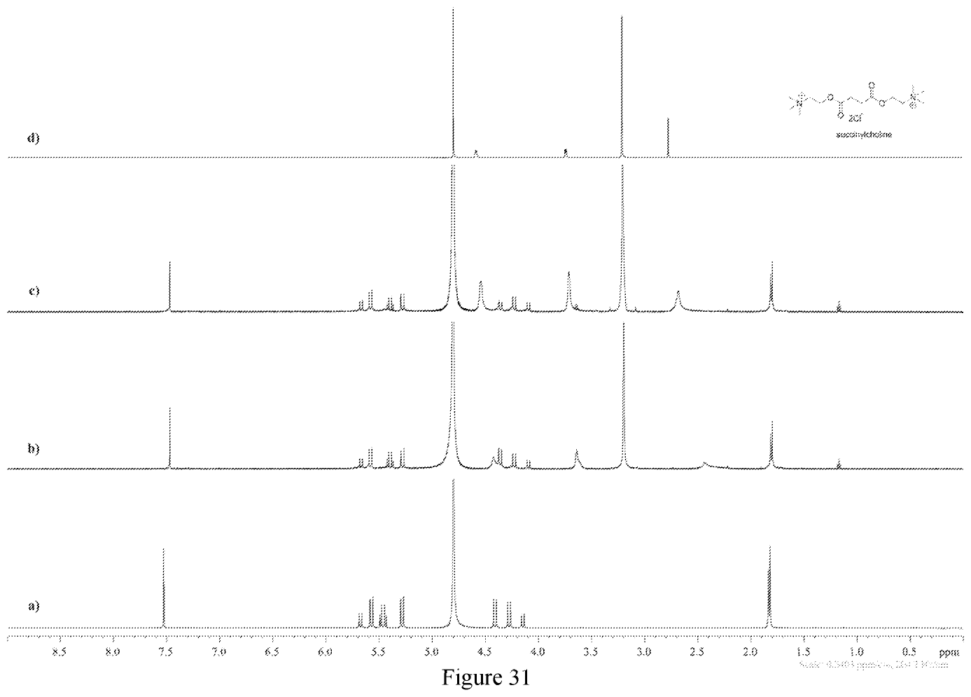

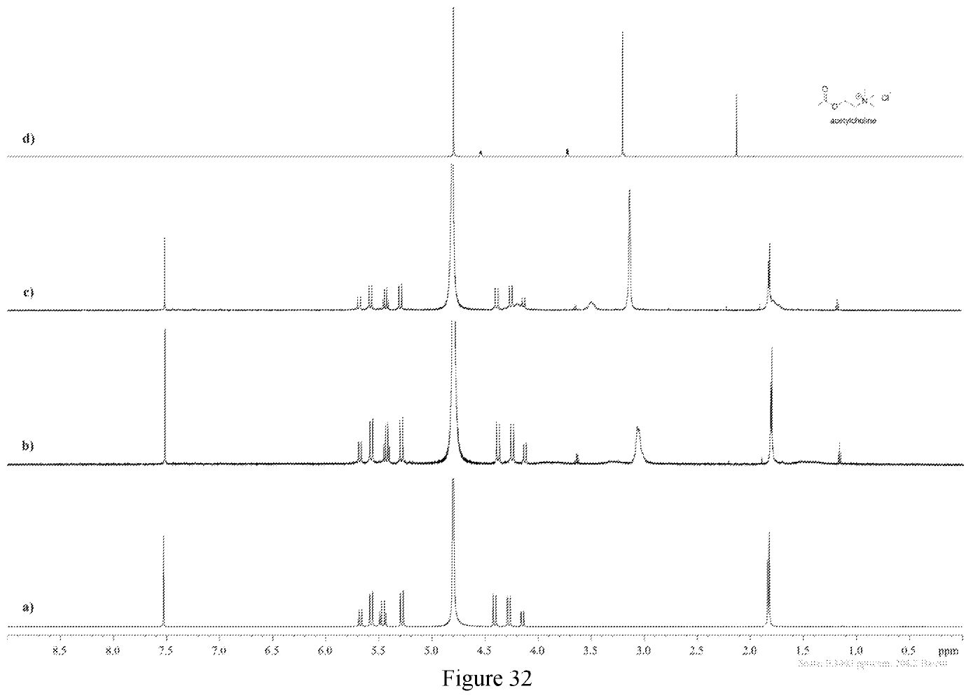

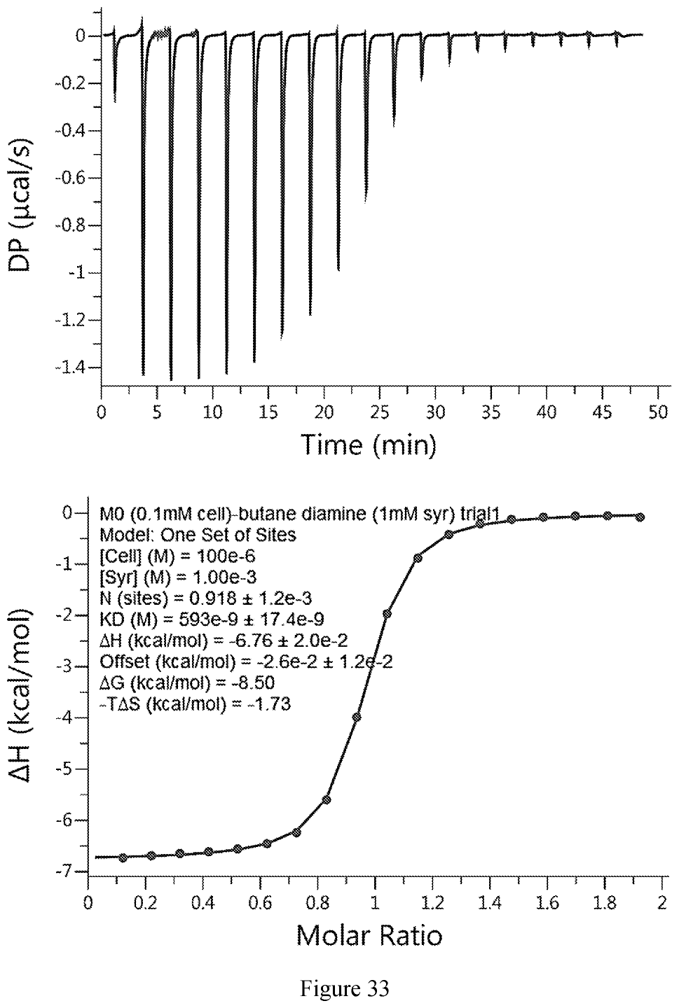

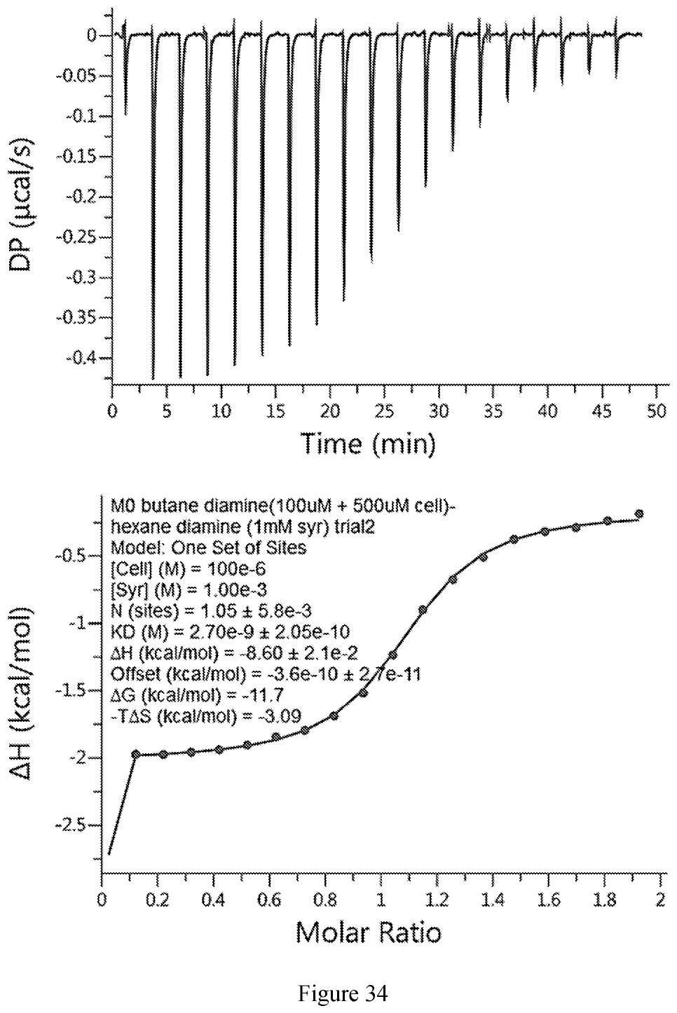

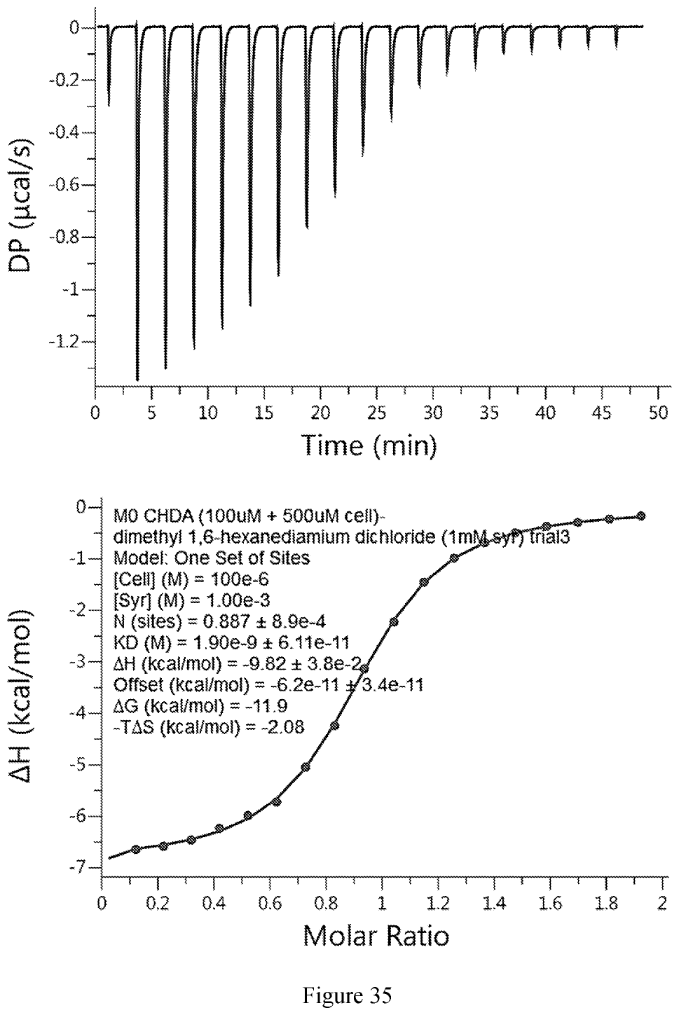

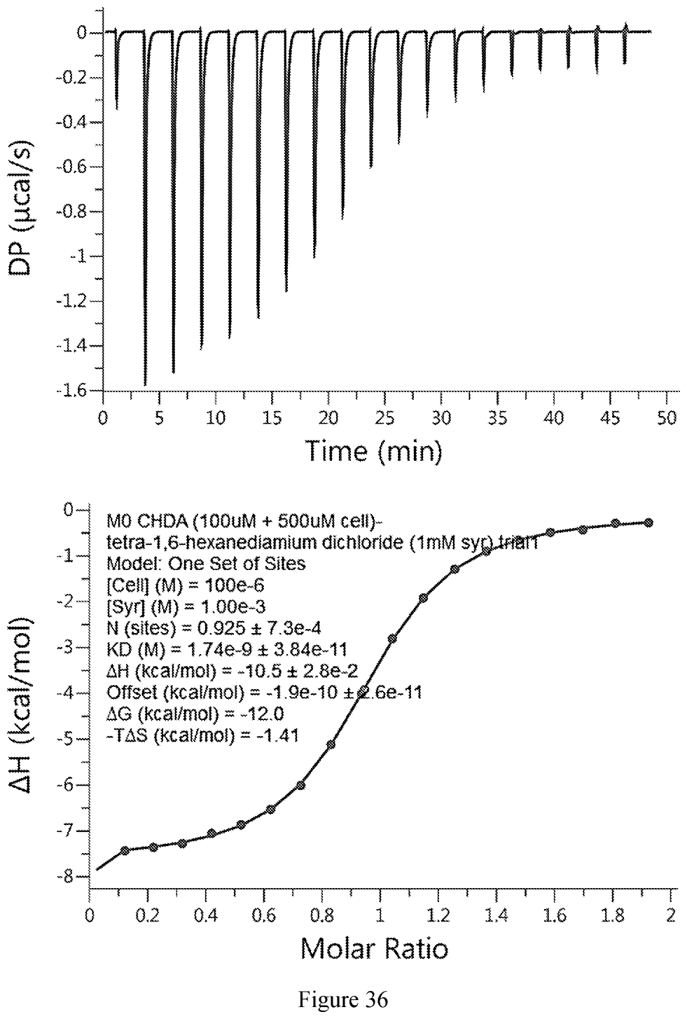

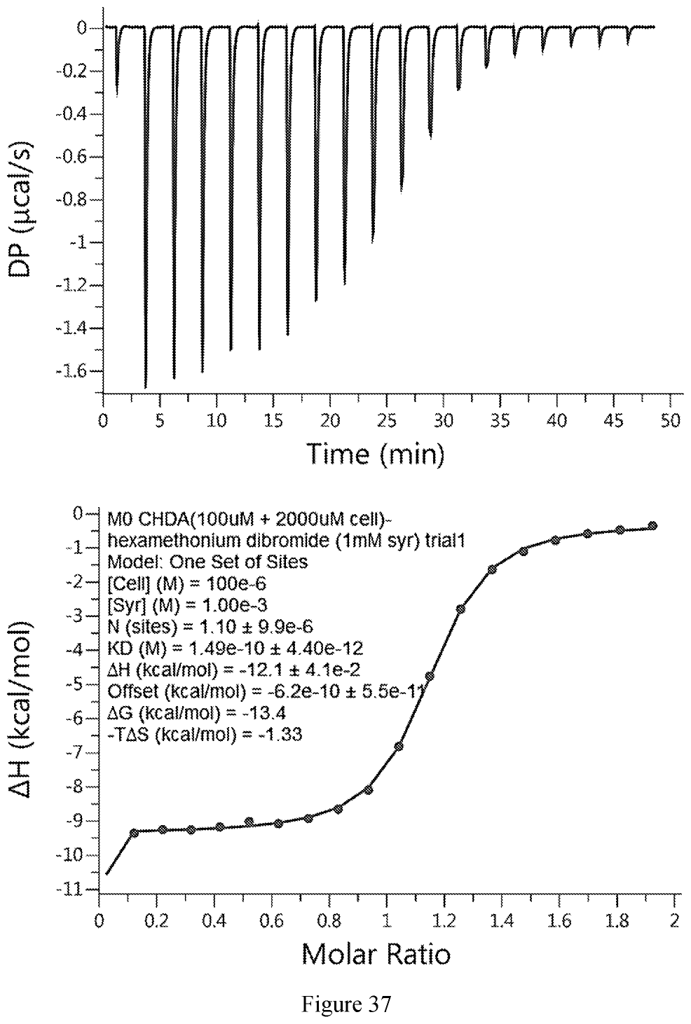

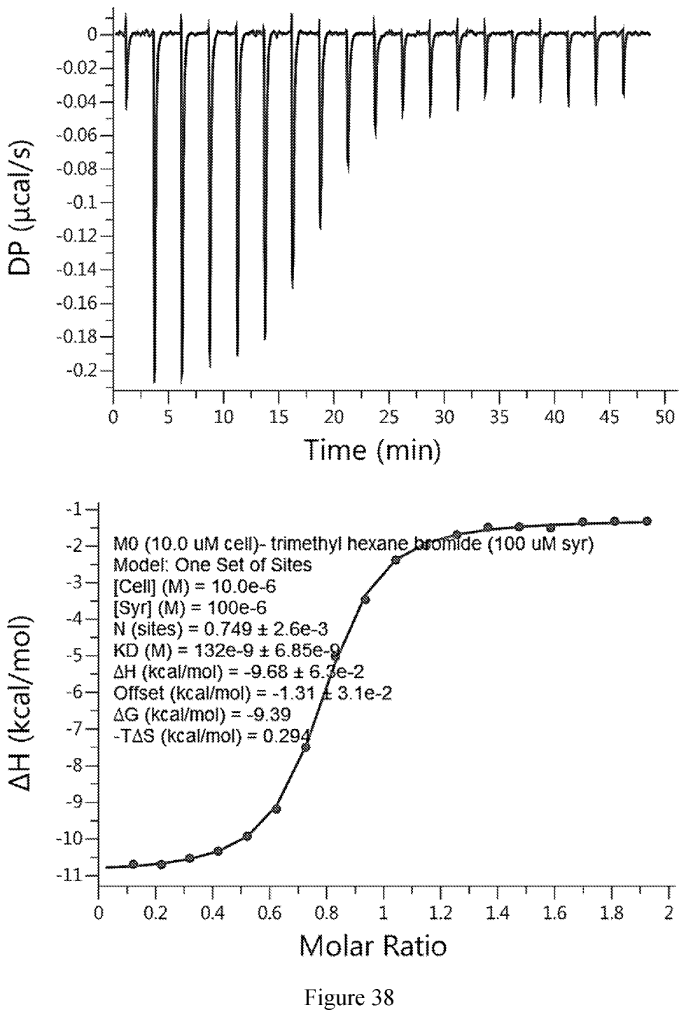

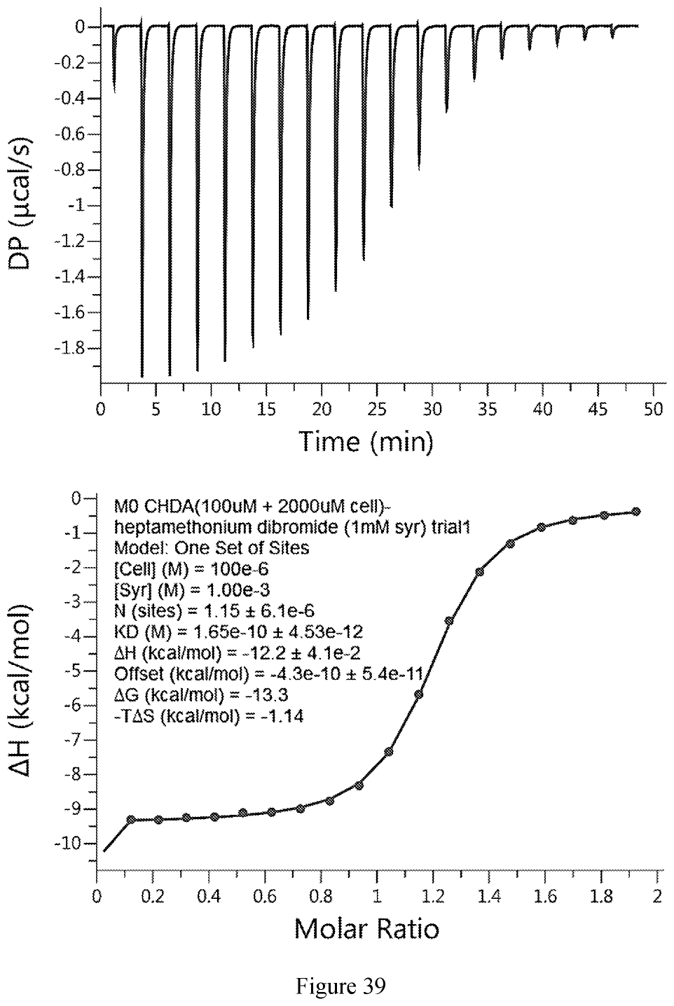

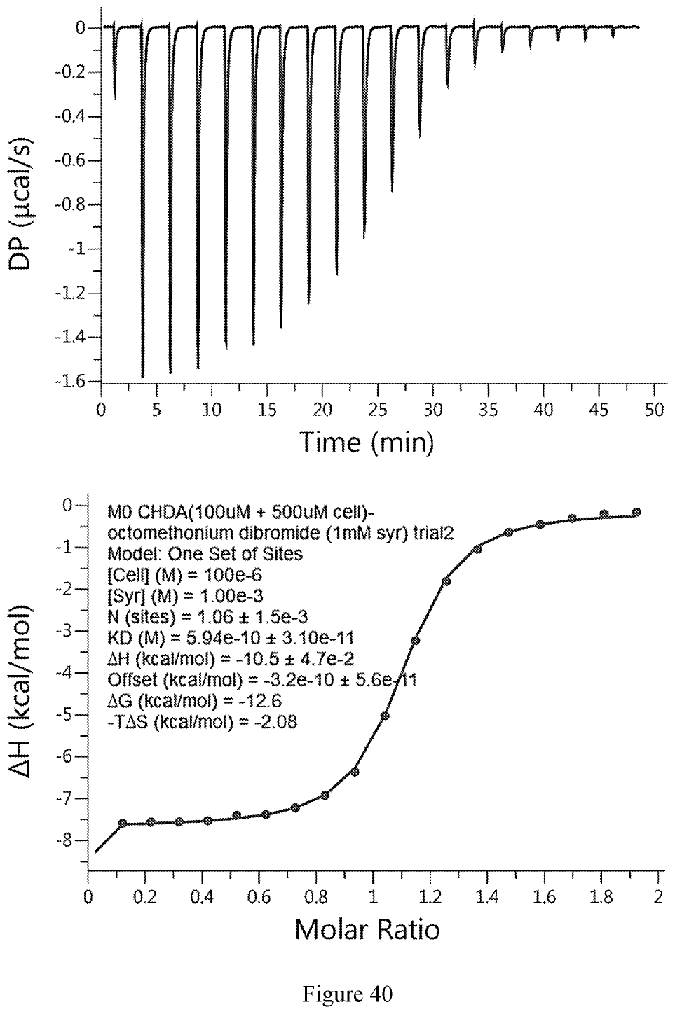

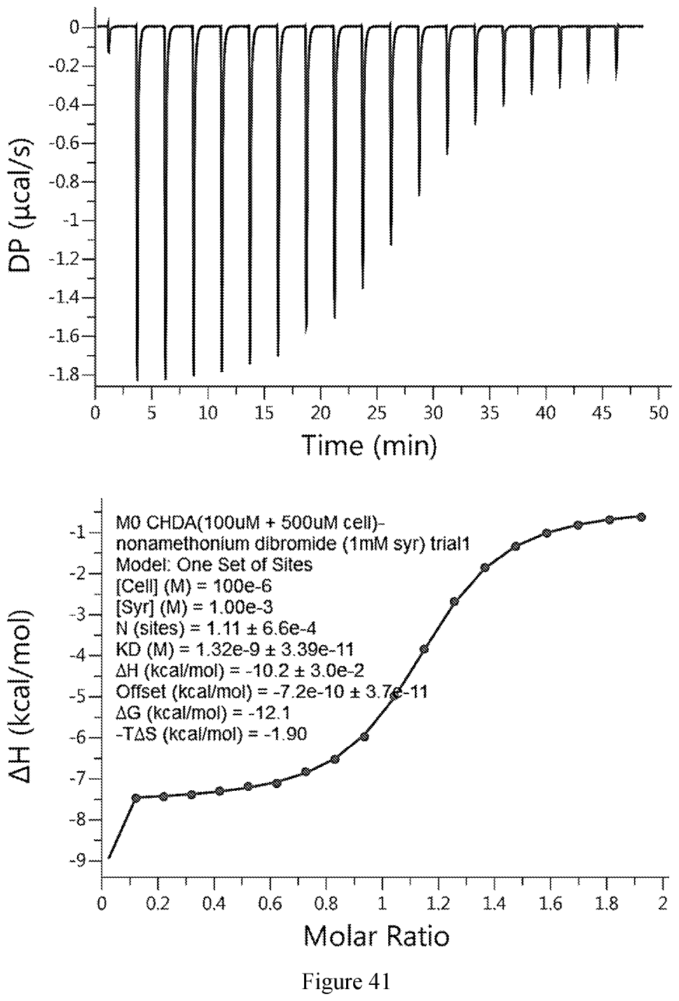

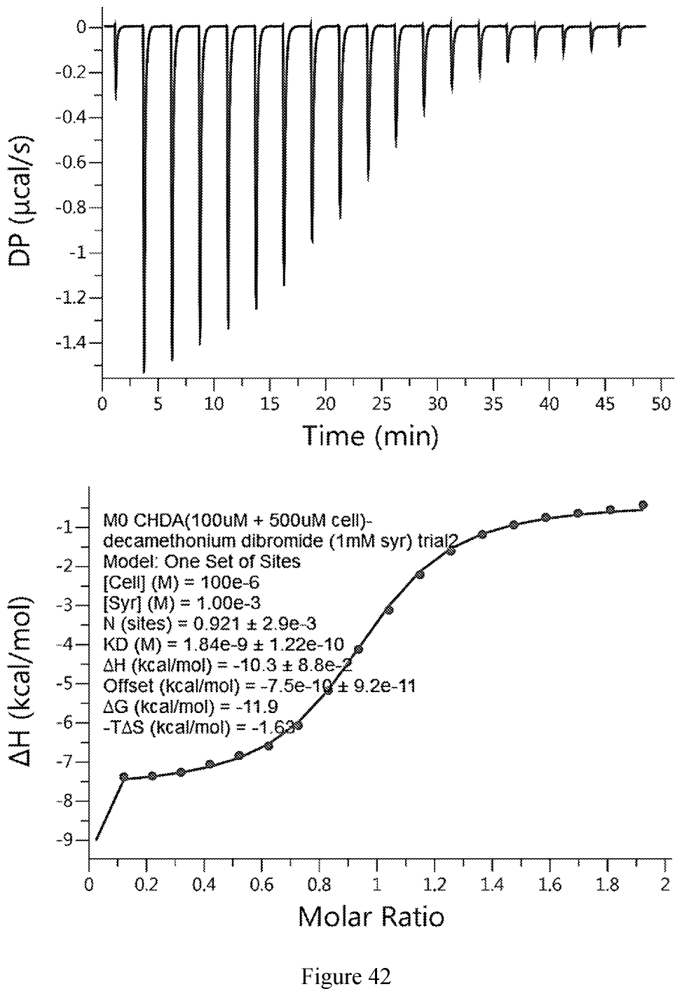

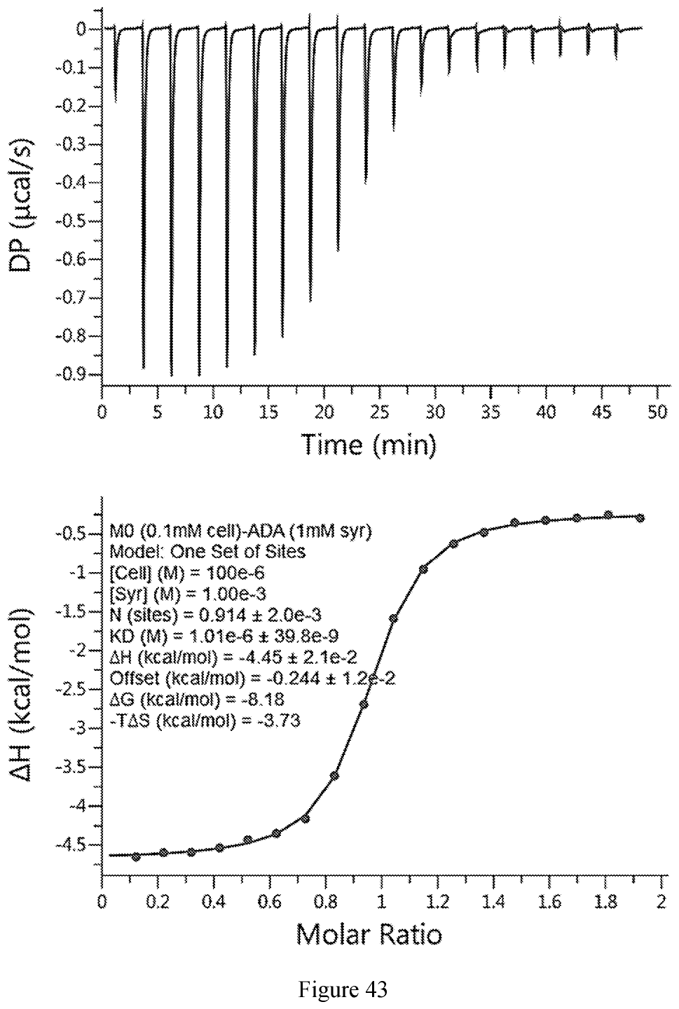

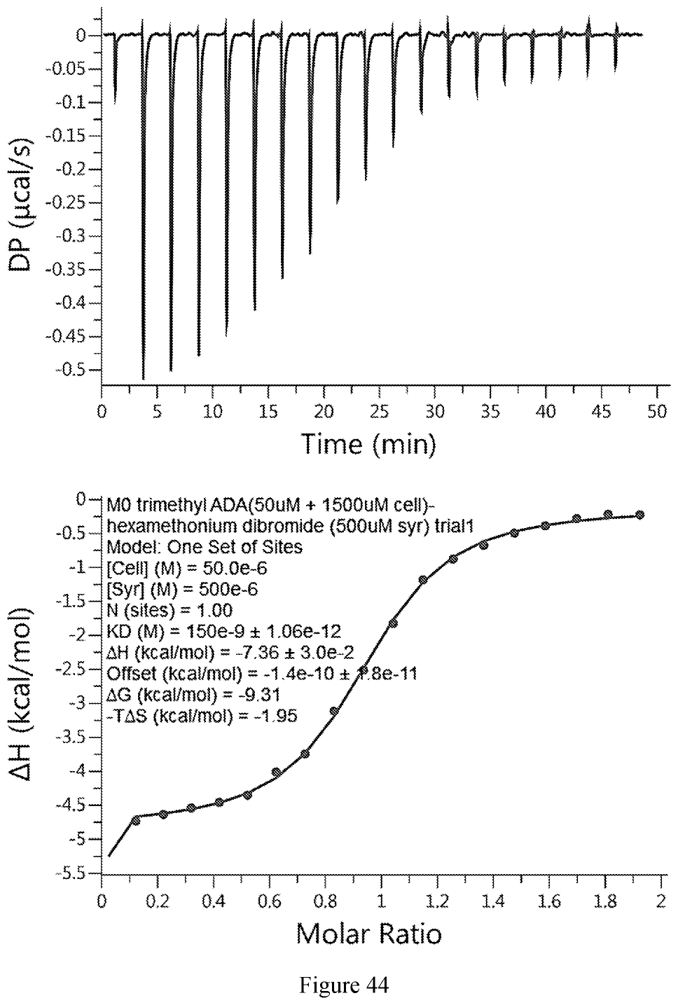

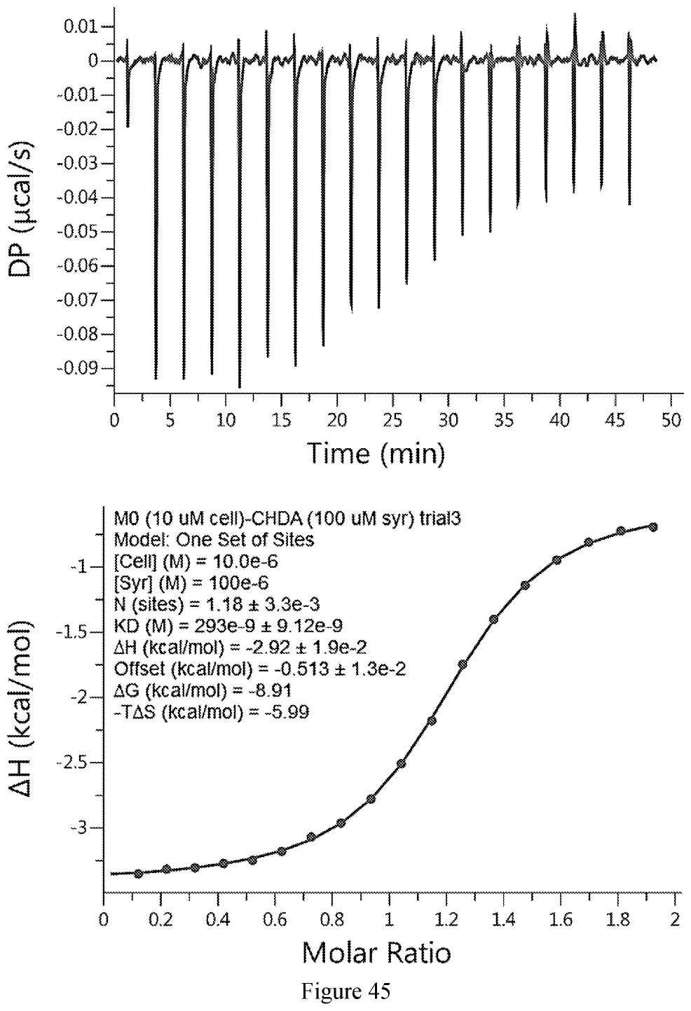

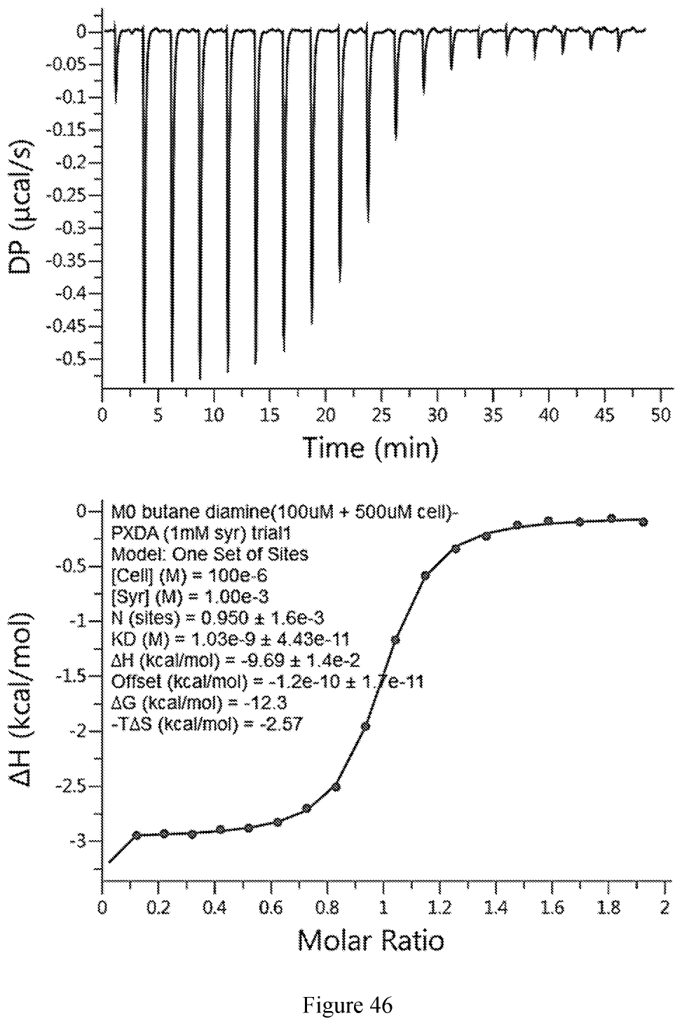

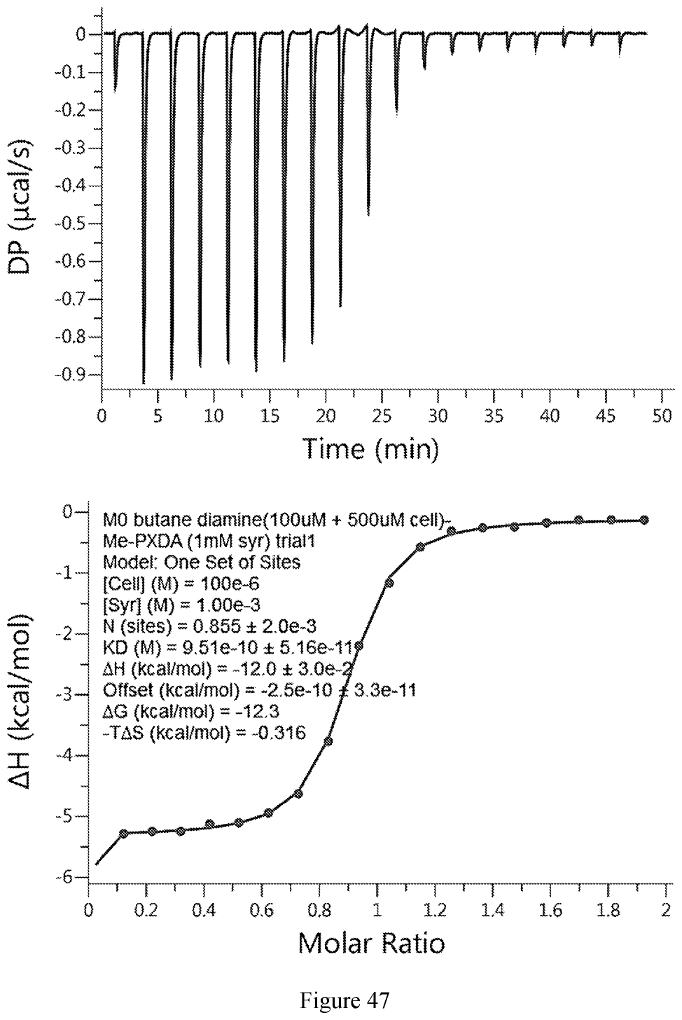

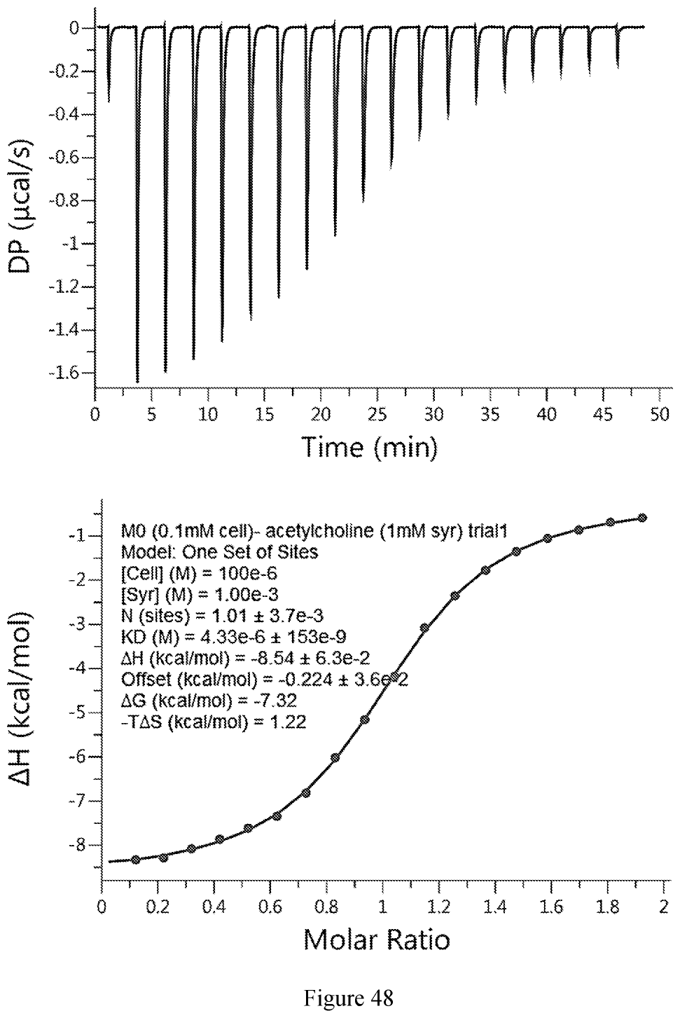

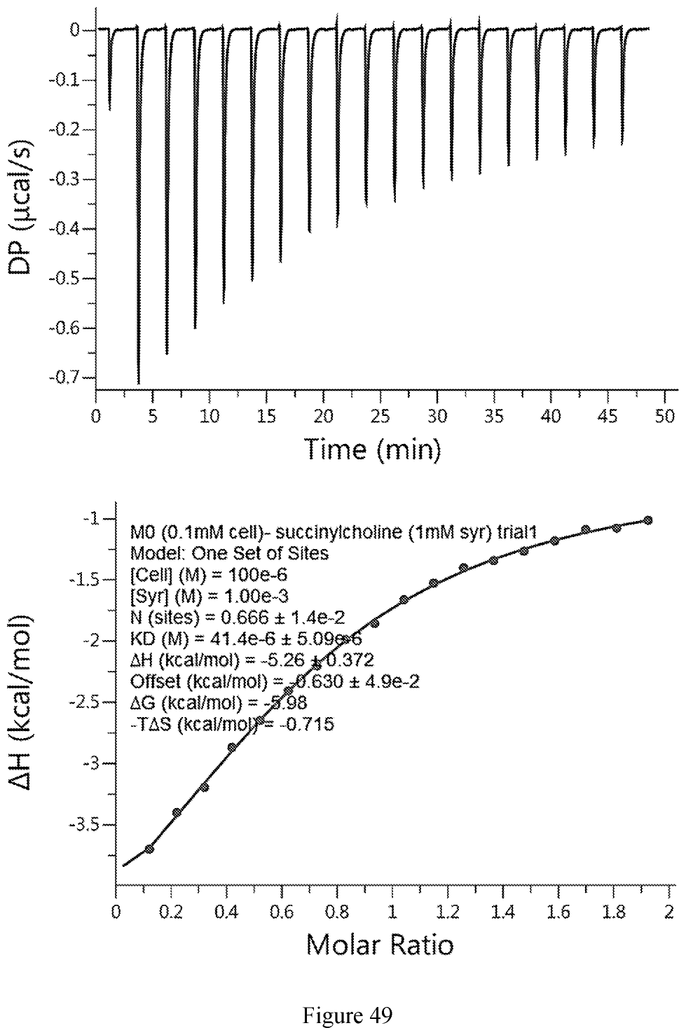

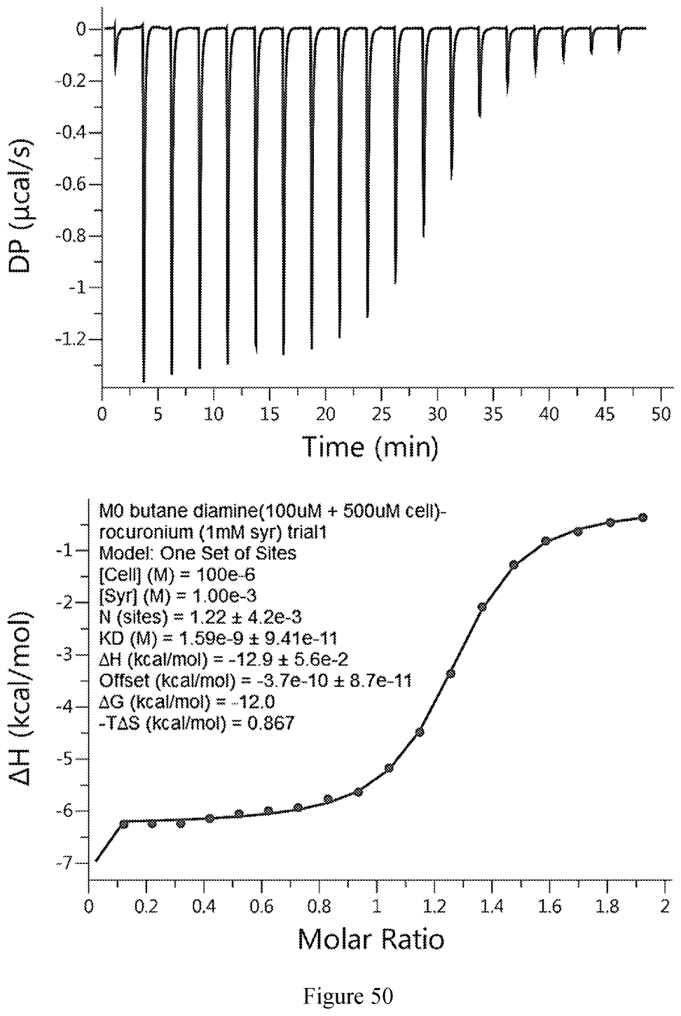

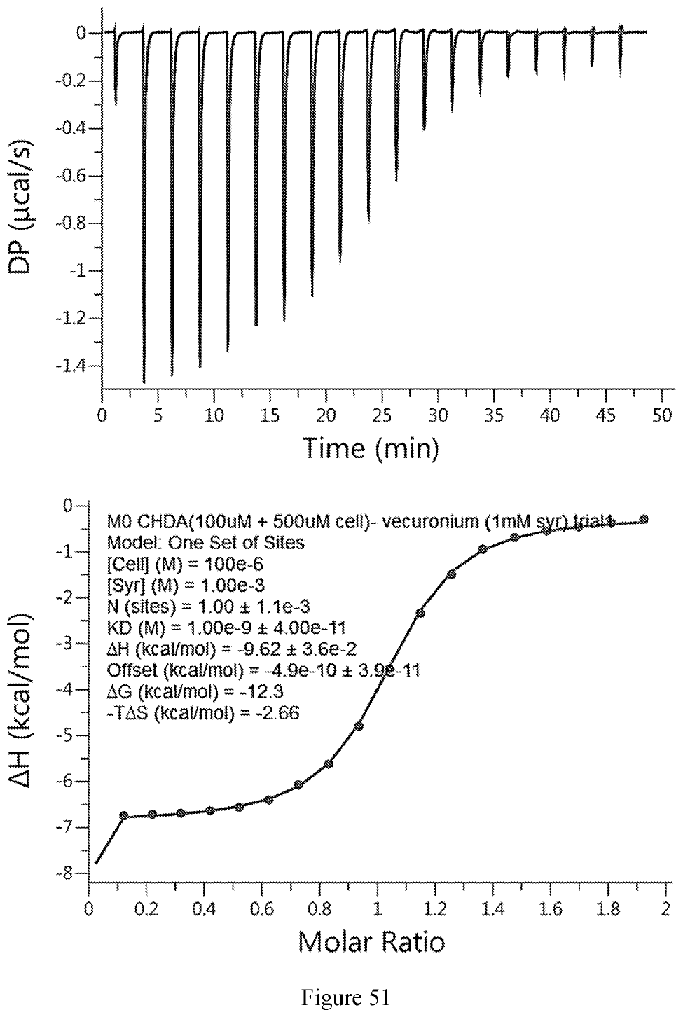

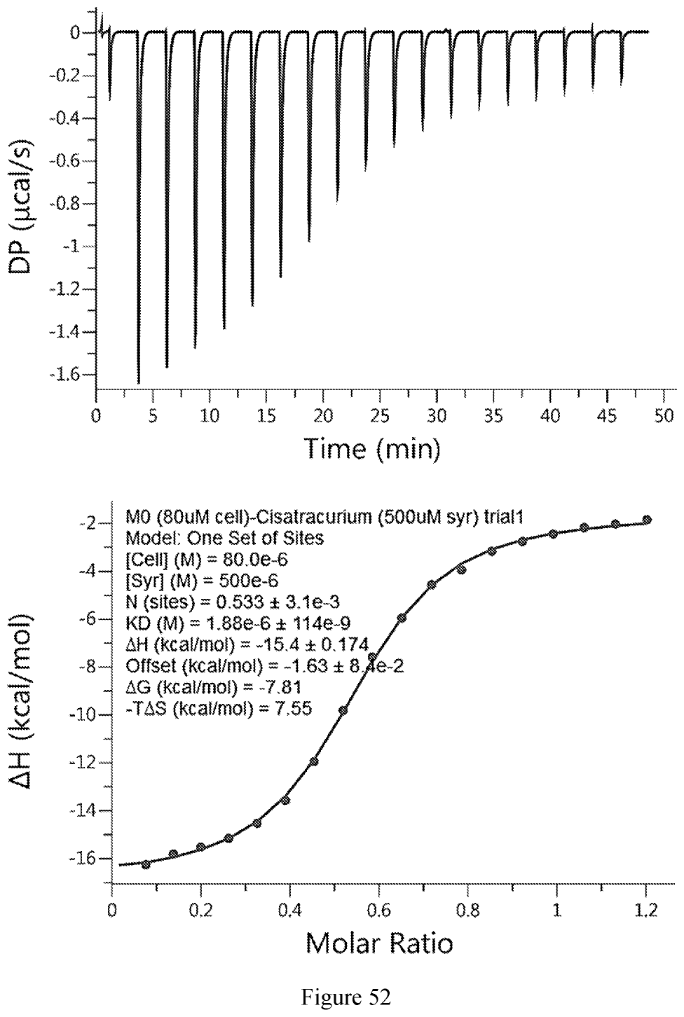

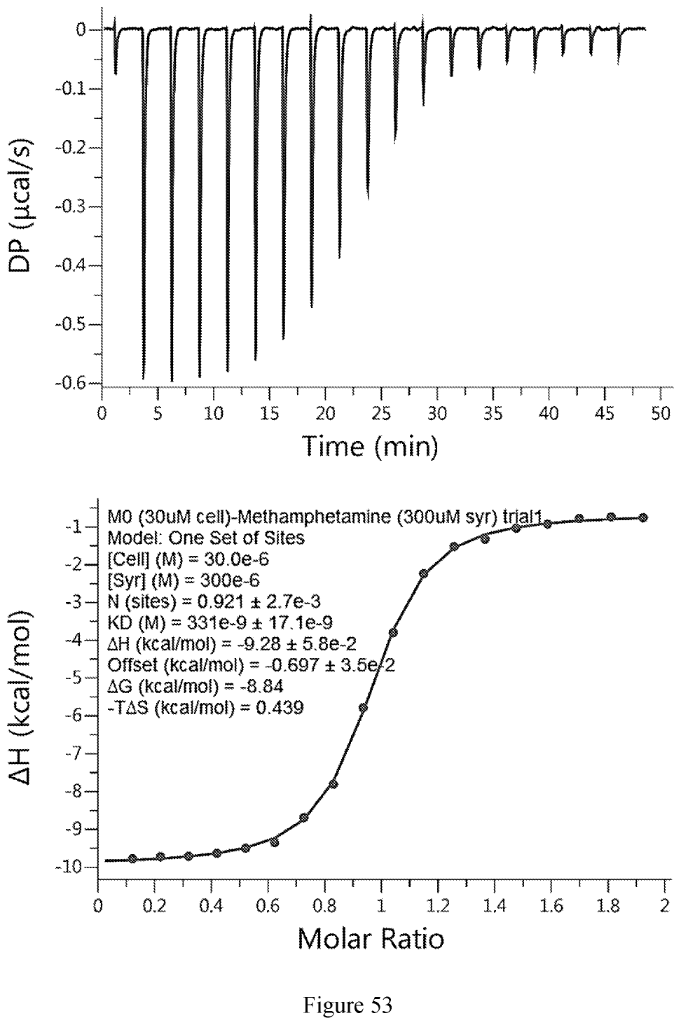

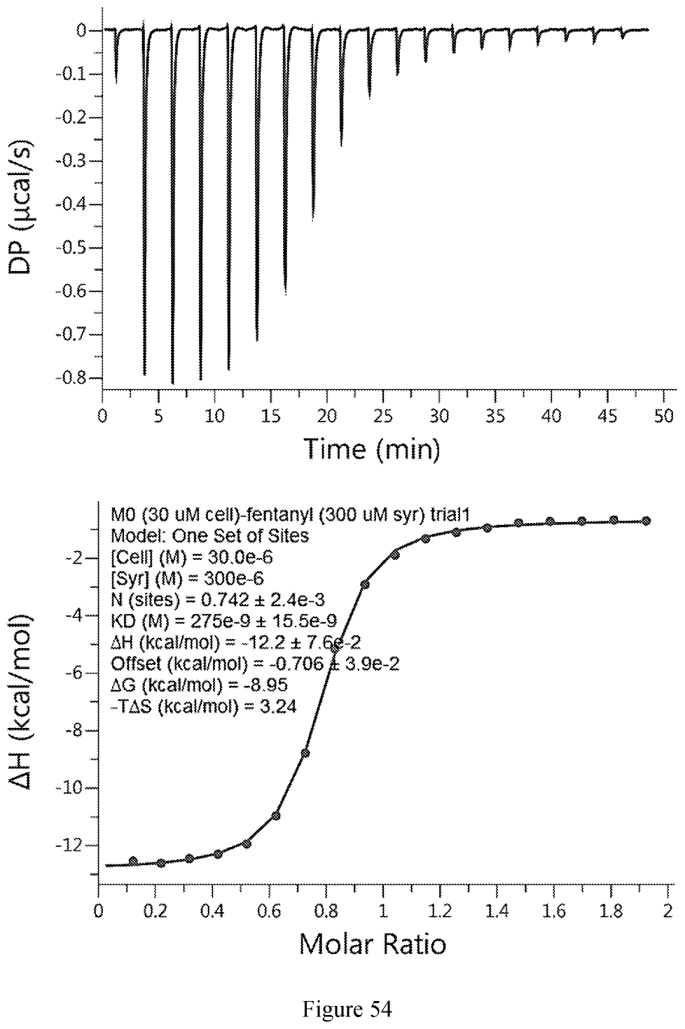

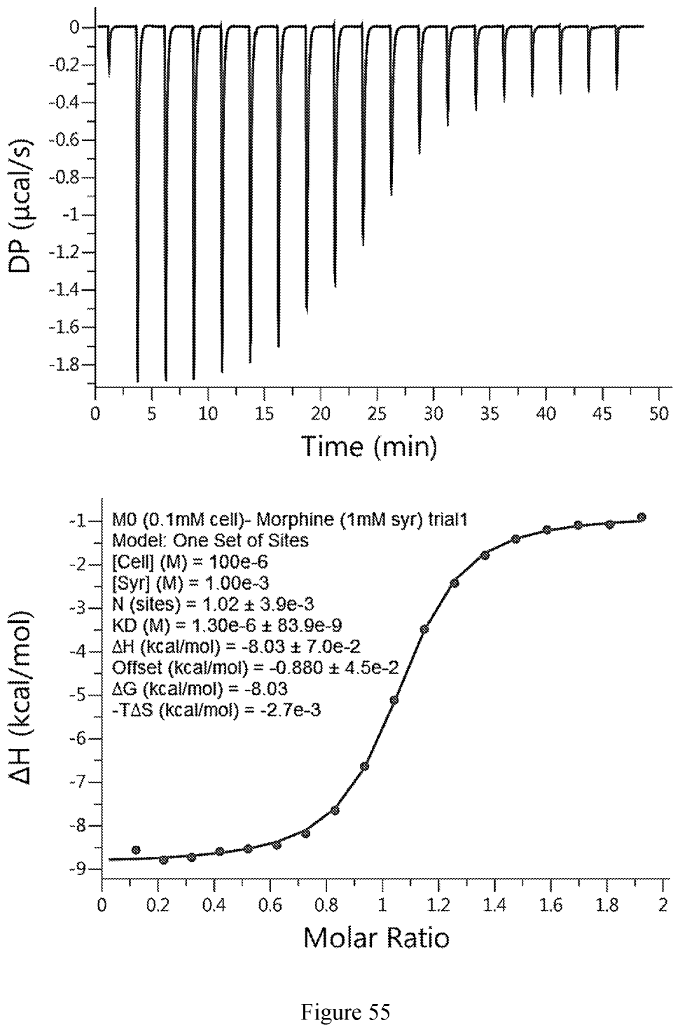

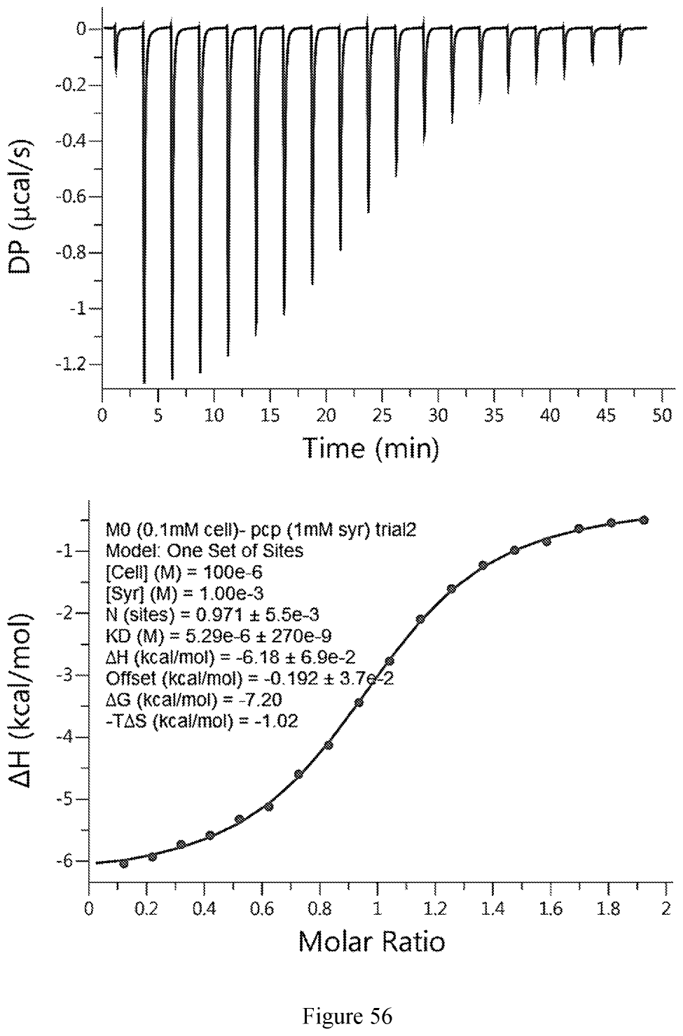

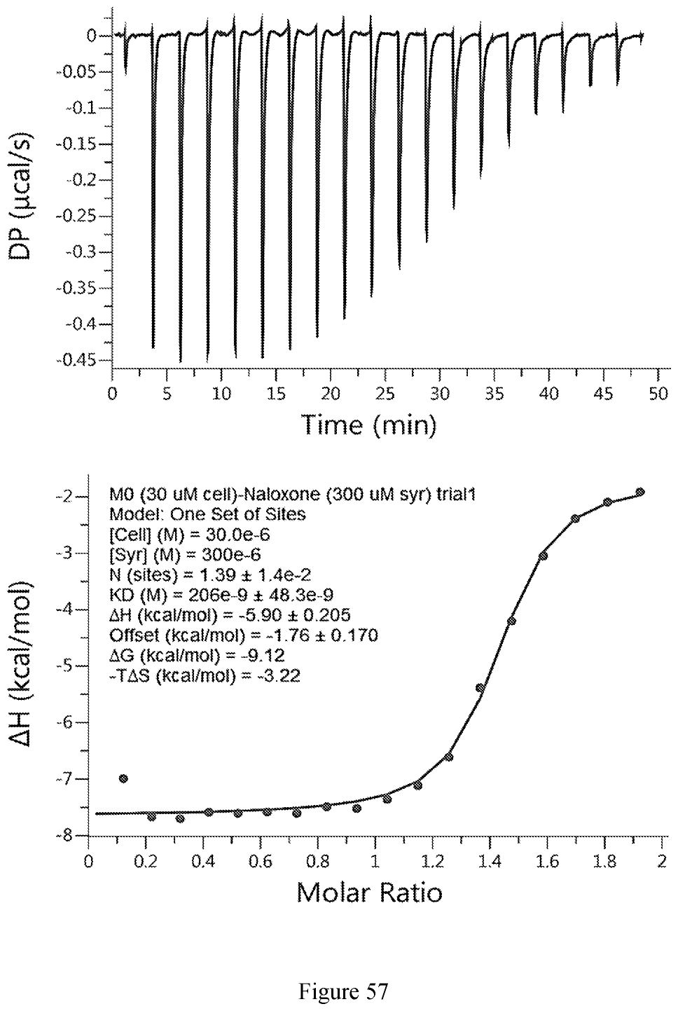

Synthetically, the key glycoluril tetramer building block (2) was allowed to react with hydroquinone (3) in TFA at room temperature to deliver hydroxylated host 4 in 99% yield on a 20 gram scale. Subsequently, 4 was allowed to react with pyridine-SO₃ complex in pyridine at 90° C. to deliver host 1 in 60% yield on a 0.5 gram scale after purification by gel permeation chromatography. Host 1 was fully characterized by spectroscopic means but its structure was also confirmed by x-ray crystallographic measurements of its host⋅guest complexes (vide infra). For example, the ¹H NMR spectrum of 1 shows six resonances for the diastereotopic methylene groups on the glycoluril oligomer backbone in the expected 4:4:4:4:2:2 ratio (H_{b}, H_{c}, H_{d}, Hₑ, H_{f}, H_{g}) along with a singlet for the aromatic H-atoms (Hₐ), two Me resonances (j and k), and two glycoluril methine resonances (Hₕ, Hᵢ) which is consistent with the depicted C₂ᵥ-symmetric structure of 1. In the ¹³C NMR we observe all 14 resonances expected on the basis of the C₂ᵥ-symmetric structure depicted for 1. Finally, the electrospray ionization mass spectrum for 1 as its complex with guest 6d exhibits a doubly charged ion at m/z 787.1679 ([M+6d-2Cl⁻]²⁺), calculated for C₅₄H₇₀N₁₈O₂₄Na₄²⁺ 787.1642. Host 1 exhibits desirable solubility in water (>40 mM). Before proceeding to investigate the host⋅guest properties of 1 its self-association properties were investigated to ensure that self-association does not impinge upon the planned binding constant measurements. Accordingly, the ¹H NMR spectrum was measured for aqueous solutions of 1 upon dilution from 40 mM to 1 mM (FIG. 13). Significant changes in chemical shifts (Δδ<0.02 ppm) were not observed over this concentration range and therefore it was concluded that host 1 is monomeric in aqueous solution.

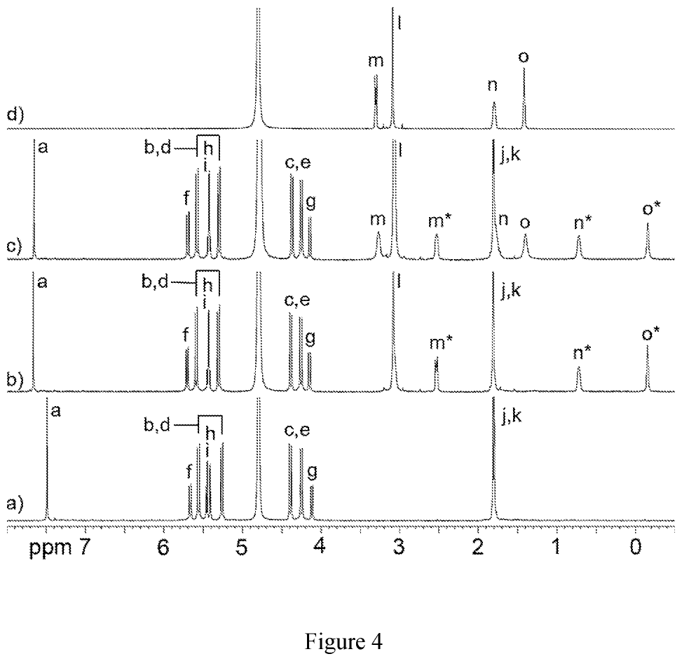

Next, qualitative host⋅guest binding studies of 1 with guests 5-13 (Chart 1) were performed and monitored by ¹H NMR spectroscopy. FIG. 4 shows the ¹H NMR spectra recorded for 1, 6d, and 1:1 and 1:2 mixtures of 1:6d. The methylene resonances for guest 6d (Hₘ, Hₙ, Hₒ) within the 1⋅6d complex (FIG. 4b) experienced a sizable upfield shift upon complexation due to the anisotropic shielding effects of the aromatic walls and the glycoluril concavity. At a 1:2 1:6d ratio, resonances were observed for both free 6d and 1⋅6d, which indicates slow exchange on the ¹H NMR chemical shift time scale which is usually observed only for tight host⋅guest complexes. Similar ¹H NMR measurements were made for the remainder of the guests. The narrow guests (e.g., 11a,d and 6a-d) displayed slow exchange kinetics, whereas the bulkier guests 12a,d displayed intermediate to fast exchange on the chemical shift timescale. This is attributed to their lower binding constants (vide infra) as a result of the expansion of the cavity of 1 required to accommodate the larger adamantane framework.

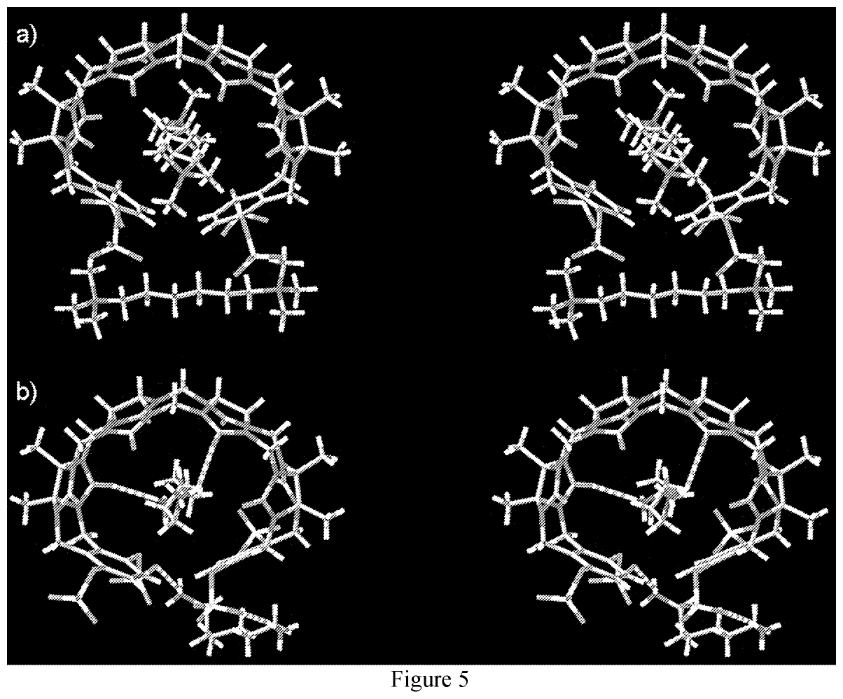

The x-ray crystal structures were obtained for 1⋅6d and 1⋅6a complexes (FIG. 5). FIG. 5a shows the 1⋅6d complex adopts a geometry that optimizes Me₃N⁺⋅⋅⋅O═C electrostatic interactions at both portals and displays only small out-of-plane skewing of the terminal aromatic rings. The geometry of 1⋅6d is reminiscent of those of CB[n]⋅guest complexes where the Me₃N⁺⋅⋅⋅O═C distances cluster in the 3.810-4.690 Å range to spread the positive charge to the carbonyl portals. Quite interestingly, a second molecule of dicationic guest 6d fits nicely into a cleft created by the aromatic sidewalls and the outward pointing OSO₃⁻ groups to balance the overall 4-charge of host 1. These OSO₃⁻ groups also engage in electrostatic interactions with 6d with the Me₃N⁺⋅⋅⋅O—S shortest distances in the 3.808-4.722 Å range. The crystal structure of 1⋅6a (FIG. 5b) also showed one intracavity and one extracavity molecule of 6a but displays significant out-of-plane twisting of the aromatic termini. Interestingly, one of the four OSO₃⁻ groups turned inward toward the ammonium ion guest which established that this group can directly participate in the guest recognition process.

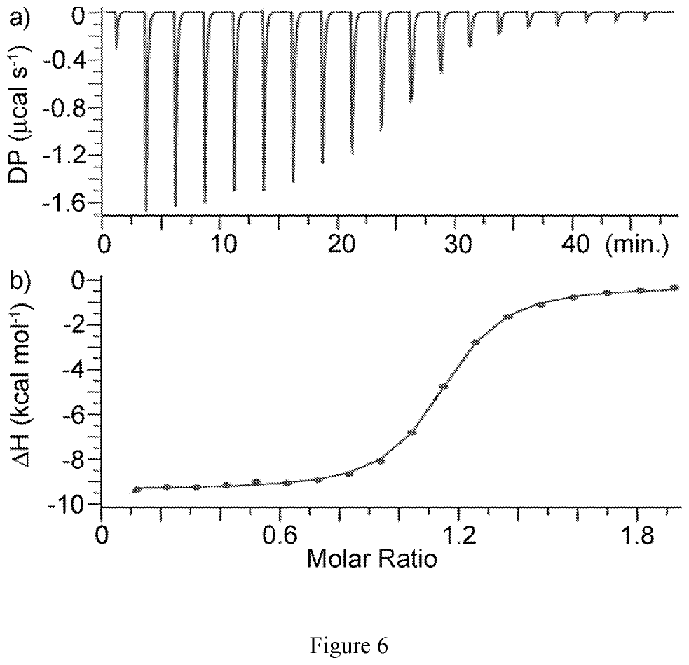

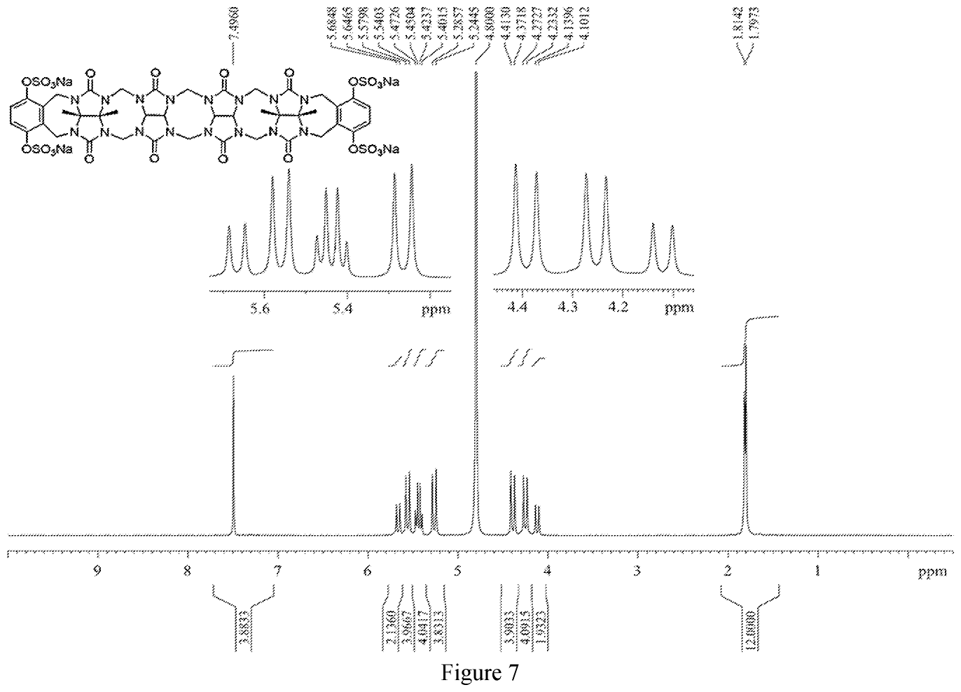

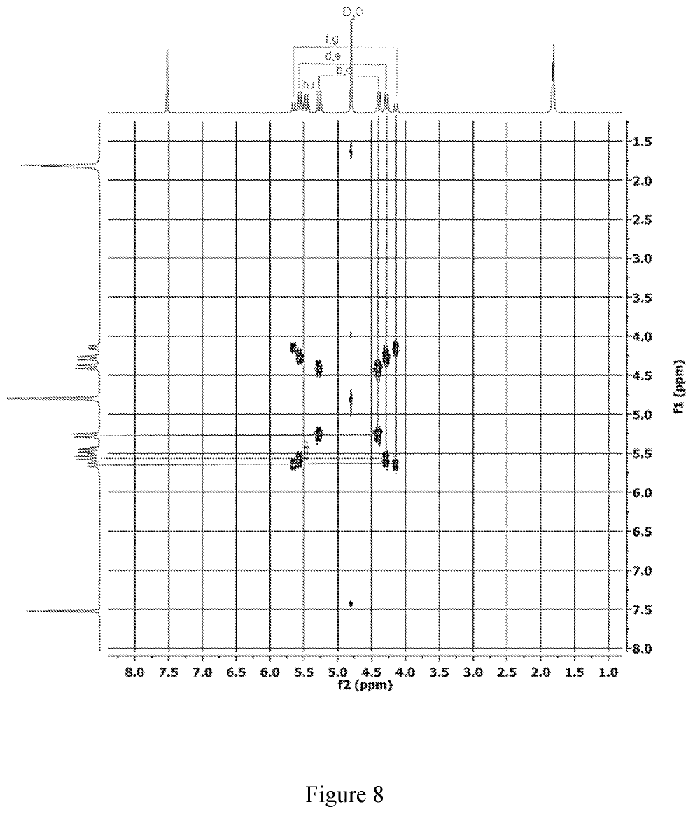

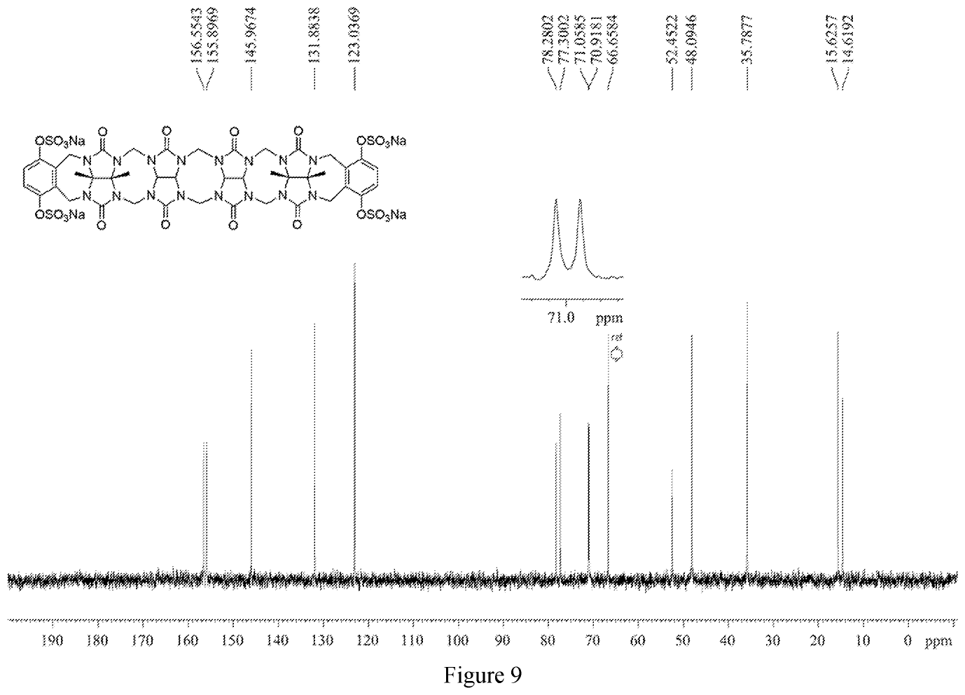

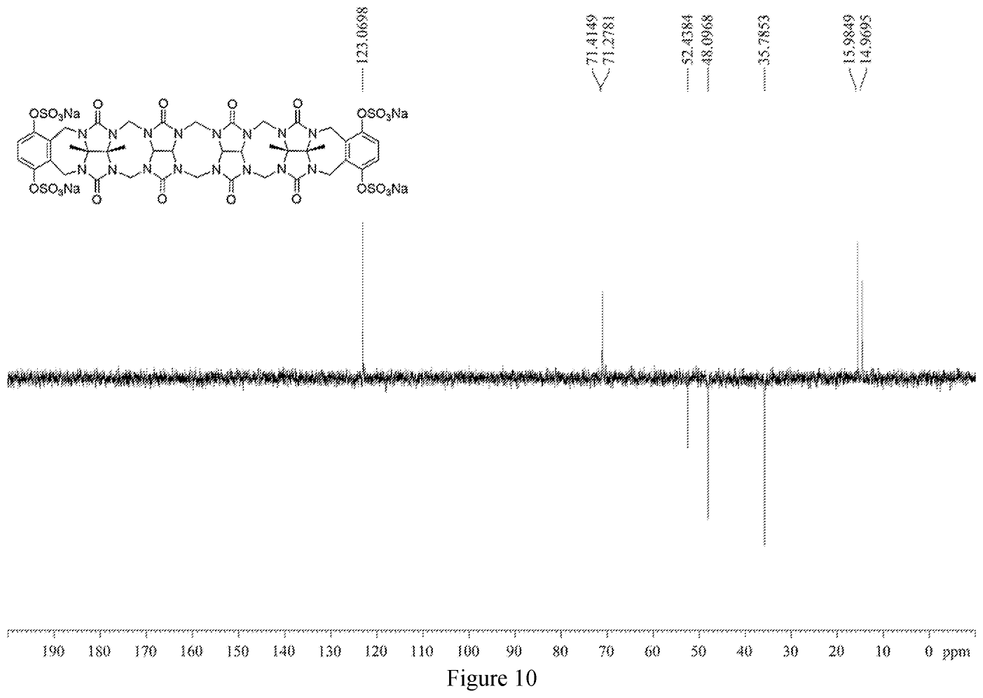

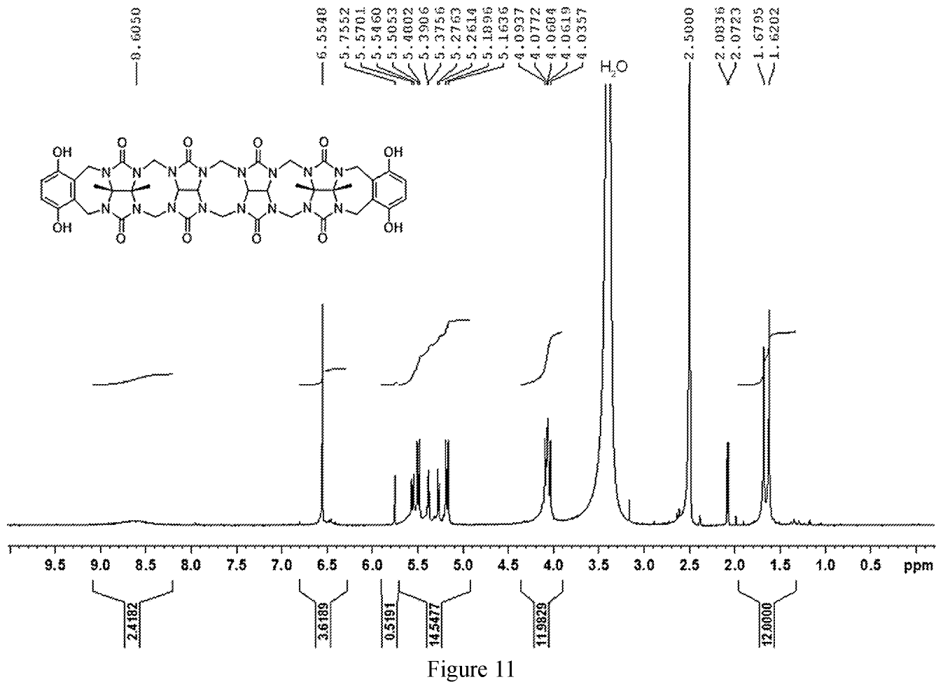

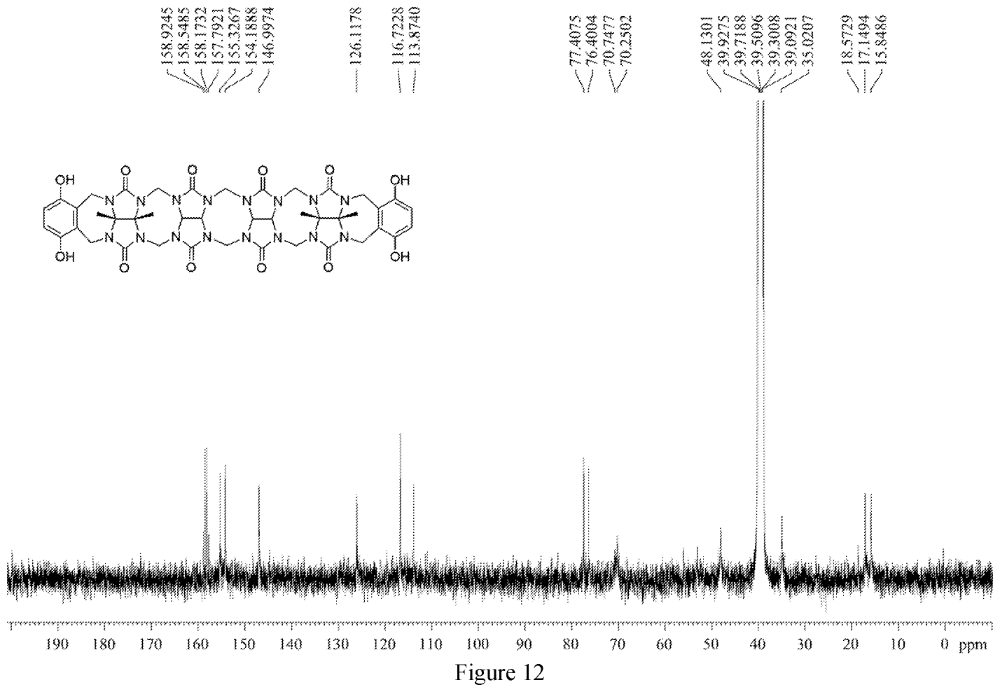

Given the high binding constants typically observed for host⋅guest complexes of CB[n] and acyclic CB[n]-type receptors, it was elected to use isothermal titration calorimetry (ITC) to measure the Kₐ values between host 1 and guests 5-23. For the weaker binding complexes (Kₐ≤10⁷ M⁻¹), the direct titration of host 1 in the ITC cell was performed with a solution of guest in the syringe and fitted the data to a 1:1 binding model implemented by the PEAQ ITC software to obtain Kₐ and ΔH values (kcal mol⁻¹). Table 1 reports the thermodynamic data for 1⋅5, 1.6Q, 1⋅12a, 1⋅13a, 1⋅14-1⋅18, and 1⋅21-1⋅23 that were obtained by direct ITC titrations. Complexes with Kₐ values that exceed 10⁷ M⁻¹ cannot be measured accurately by direct titrations, so ITC competitive titrations were used. In competitive titrations, a solution of host and an excess of a weak guest of known ΔH and Kₐ was titrated with a solution of a tighter binding guest. Fitting of the heat released during the displacement process is analyzed by a competitive binding model in the PEAQ ITC data analysis software which delivers ΔH and Kₐ for the tighter binding complex. FIG. 6a shows the thermogram recorded when a mixture of 1 and 13 was titrated with 6d and FIG. 6b shows the fitting of the integrated heats to a competitive binding model to determine Kₐ=6.79×10⁹ M⁻¹ and ΔH=−12.1 kcal mol⁻¹. Table 1 reports Kₐ and ΔH values for the remaining 1⋅guest complexes obtained in an analogous manner.

TABLE 1
Binding constants measured by ITC for host · guest complexes of 1
Comparative data for M1 are drawn from the literature. Conditions:
20 mM sodium phosphate buffered H₂O, pH 7.4, 25° C..

| | Kₐ [M⁻¹]; ΔH (kcal mol⁻¹) | |
|---|---|---|
| | 1 | M1^{e)} |
| 5 | 1.68 × 10⁶; −6.76 ± 0.020^{a)} | — |
| 6a | 3.70 × 10⁸; −8.60 ± 0.021^{b)} | 5.05 × 10⁷; −6.23 ± 0.014 |
| 6b | 5.26 × 10⁸; −9.82 ± 0.038^{c)} | 9.43 × 10⁷; −7.15 ± 0.025 |
| 6c | 5.74 × 10⁸; −10.5 ± 0.028^{c)} | 4.81 × 10⁷; −7.66 ± 0.073 |
| 6d | 6.71 × 10⁹; −12.1 ± 0.042^{c)} | 8.93 × 10⁷; −9.35 ± 0.021 |
| 6Q | 7.57 × 10⁶; −9.68 ± 0.063^{a)} | 1.24 × 10⁶; −5.67 ± 0.033 |
| 7d | 6.06 × 10⁹; −12.2 ± 0.041^{c)} | — |
| 8d | 1.75 × 10⁹; −10.5 ± 0.032^{c)} | — |
| 9d | 7.57 × 10⁸; −10.2 ± 0.030^{c)} | — |
| 10d | 5.43 × 10⁸; −10.3 ± 0.088^{c)} | — |
| 11a | 9.71 × 10⁸; −9.69 ± 0.014^{b)} | 1.67 × 10⁸; −8.09 ± 0.018 |
| 11d | 1.05 × 10⁹; −12.0 ± 0.030^{b)} | 1.78 × 10⁸; −11.4 ± 0.022 |
| 12a | 9.90 × 10⁵; −4.45 ± 0.021^{a)} | 9.62 × 10⁵; −6.55 ± 0.029 |
| 12d | 6.66 × 10⁶; −7.36 ± 0.030^{d)} | 1.70 × 10⁷; −9.09 ± 0.027 |
| 13a | 3.41 × 10⁶; −2.92 ± 0.019^{a)} | 1.95 × 10⁶; −5.70 ± 0.027 |
| 14 | 3.02 × 10⁶; −9.28 ± 0.058^{a)} | 7.5 × 10⁶ |
| 15 | 3.64 × 10⁶; −12.2 ± 0.076^{a)} | 1.1 × 10⁷ |
| 16 | 1.89 × 10⁵; −6.18 ± 0.069^{a)} | 4.7 × 10⁴ |
| 17 | 7.69 × 10⁵; −8.03 ± 0.07^{a)} | 5.3 × 10⁵ |
| 18 | 4.85 × 10⁶; −5.90 ± 0.205^{a)} | — |
| 19 | 6.29 × 10⁸; −12.9 ± 0.056^{b)} | 8.4 × 10⁶ |
| 20 | 1.00 × 10⁹; −9.62 ± 0.036^{c)} | 5.8 × 10⁶ |
| 21 | 5.32 × 10⁵; −15.4 ± 0.174^{a)} | 9.7 × 10⁵ |
| 22 | 2.41 × 10⁴; −5.26 ± 0.372^{a)} | — |
| 23 | 2.31 × 10⁵; −8.54 ± 0.063^{a)} | 2.4 × 10⁴ |

— not reported in the literature.
^{a)}Direct titration,
^{b)}competitive ITC with 5 as competitor,
^{c)}competitive ITC with 13a as competitor,
^{d)}competitive ITC with 6d as competitor,
^{e)}Taken from the literature.

The binding constant data reported in Table 1 allowed conclusions to be drawn about the molecular recognition preferences of host 1 in comparison to M1. It was found that the 1⋅guest complexes are uniformly driven by favorable enthalpic (ΔH) contributions. In the CB[n] series of hosts these favorable enthalpy values are attributed to the presence of high energy host intracavity water molecules that are released upon guest binding. Host 1 displays high affinity toward hexanediammonium ion guests 6a-6d with K_{d} values in the single digit nM to sub-nM range. Host 1 prefers the quaternary ammonium ion guest 6d by ≈10-fold over the primary-tertiary ammonium ions 6a-6c. In selected contexts, related preferences were seen for CB[7] where they are attributed to the more efficient spreading of positive charge to the entire ureidyl C═O portal. Host 1 binds quaternary monoammonium ion guest 6Q 890-fold weaker than the corresponding quaternary diammonium 6d; this ≈10³ M⁻¹ difference in affinity is also noted for CB[n]-type receptors. It was found that 1 binds to guests 6a-6c 5.6-11.9-fold stronger than M1, but 75-fold stronger than M1 toward bis(quaternary) guest 6d. Similar preferences are observed for dicationic guests 11a and 11d but not for monocationic guests 12a and 12d which suggests that the defined separation between OSO₃⁻ groups in 1 makes it especially complementary to diammonium ion guests. Host 1 also binds with high affinity (single digit nM to sub nM K_{d} values) toward the longer alkanediammonium ions 7d-10d although 6d is the tightest binder in this series which reflects the ability of acyclic CB[n] to flex their cavity to accommodate larger guests and optimize binding affinity. Related preferences were seen for M1 and related receptors toward primary alkane diammonium ion guests previously.

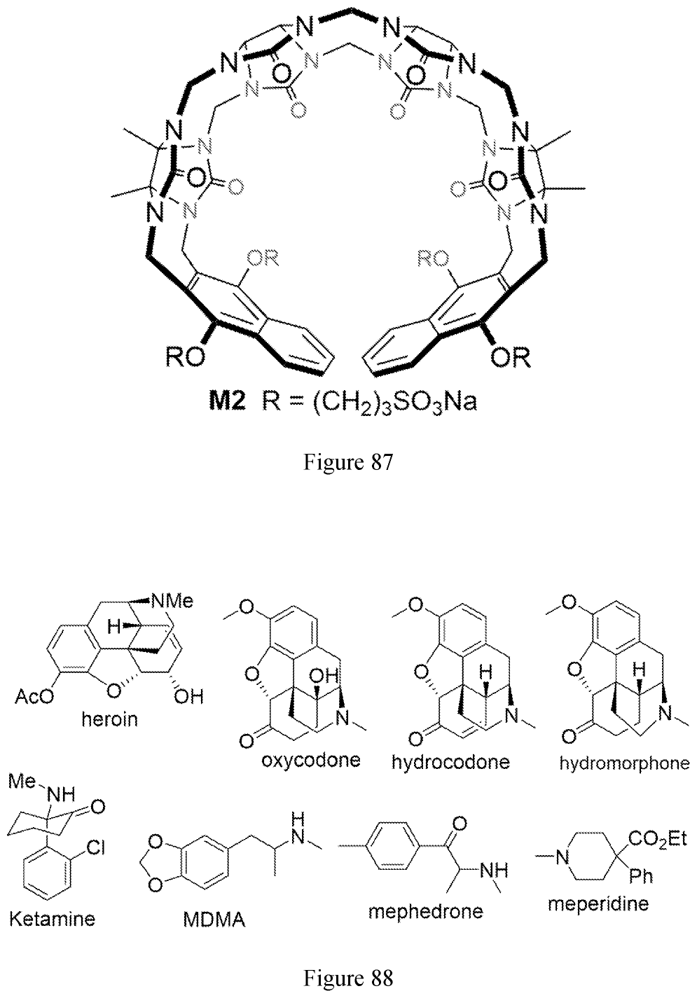

Previously, it was shown that M1 and a naphthalene walled analogue known as M2 (FIG. 87) function as in vivo sequestration agents for drugs of abuse (e.g., methamphetamine (14)). Accordingly, it was decided to measure the binding affinities of some compounds (14-18) relevant to counteracting the effects of drugs of abuse. It was found that host 1 binds less tightly than M1 toward 14 and 15. In contrast, host 1 bound somewhat tighter to PCP (16) and morphine (17) than M1 does. This is perhaps not surprising given that 1 has a distinct preference for bis(quaternary) diammonium ions whereas 14-17 are secondary and tertiary ammonium ions.

In a separate line of inquiry, it was previously shown that M1 and M2 act as in vivo reversal agents for neuromuscular block induced by rocuronium (19), vecuronium (20), and cisatracurium (21). Accordingly, the binding constants of 1 were measured toward a panel of compounds relevant to its potential use as an in vivo reversal agent. Table 1 shows that 1 possesses higher binding affinity toward 19 (75-fold) and 20 (172-fold) than M1 does. Importantly, 1 binds >2700-fold tighter to 19 or 20 than to acetylcholine (23). Acetylcholine is also present in the neuromuscular junction and must not be sequestered. The affinity of 1⋅19 (6.29×10⁸M⁻¹) and 1⋅20 (1.00×10⁹ M⁻¹) are comparable to those of M2⋅19 (3.4×10⁹ M⁻¹) and M2⋅20 (1.6×10⁹ M⁻¹) which function very well in vivo. Host 1, however, possesses superior aqueous solubility (>40 mM) compared to M2 (18 mM) which might prove advantageous for formulation purposes.

In summary, the synthesis of a new acyclic CB[n]-type receptor (1) with OSO₃⁻ groups directly connected to the aromatic walls is presented. Host 1 has excellent aqueous solubility (40 mM), does not undergo self-association, and binds more tightly to quaternary diammonium ions than analogue M1 that features propylene linking chains. The x-ray crystal structures of 1⋅6a and 1⋅6d show the usual cavity encapsulation of the diammonium guest but also show an external diammonium ion that balances the overall charge of the tetraanionic host 1. In conclusion, it was found that the negatively charged OSO₃⁻ groups do not merely function as solubilizing group, but rather their close proximity to the ureidyl C═O portals of 1 results in enhanced binding affinity toward quaternary diammonium ions including important neuromuscular blocking agents (19 and 20). This suggests that 1 should be considered alongside M1 and M2 as in vivo reversal agents for neuromuscular blockers.

General Experimental

All chemicals and reagents were purchased from commercial suppliers and were used without further purification. Compound 2 was prepared as described previously. NMR spectra were measured at 400, 500 or 600 MHz for ¹H and 100 and 125 MHz for ¹³C. The solvent for NMR experiments was deuterated water (D₂O), deuterated chloroform (CDCl₃), or deuterated dimethyl sulfoxide (DMSO-d₆). Chemical shifts (δ) are referenced relative to the residual resonances for HOD (4.80 ppm), CHCl₃ (7.26 ppm for ¹H, 77.16 ppm for ¹³C), DMSO-d₆ (2.50 ppm for ¹H, 39.51 ppm for ¹³C). Isothermal titration calorimetry was performed using a MicroCal PEAQ-ITC Isothermal Titration Calorimeter using 20 mM phosphate buffered water at pH=7.4 at 298 K. Mass spectrometry was performed using a JEOL AccuTOF electrospray instrument. Melting points were measured by a Meltemp apparatus in open capillary tubes and are uncorrected. IR spectra were measured on a Thermo Nicolet NEXUS 670 FT/IR spectrometer by attenuated total reflectance (ATR) and are reported in cm⁻¹.

Synthesis and Characterization of Compounds 4 and 1.

Container 4. To a mixture of glycoluril tetramer bisether 2 (17 g, 22.1 mmol) and hydroquinone (9.7 g, 88.2 mmol) was added trifluoroacetic acid (300 mL). The resulting heterogeneous mixture was stirred under N₂ at 25° C. for 16 hours. The reaction mixture was then poured into MeOH (600 mL) and stirred for 1 hour. The crude product was collected by filtration and subsequently washed with MeOH (400 mL), acetone (300 mL) and water (400 mL) to remove the unreacted hydroquinone. The residue was dried under the high vacuum to yield 2 as a pale yellow solid (20.8 g, yield 99%). M.p.>300° C., IR (ATR, cm⁻¹): 3400w, 1713s, 1460s, 1226s, 1082m, 971w, 797s. ¹H NMR (DMSO-d₆, 600 MHz): δ 8.60 (s, 4H), 6.55 (s, 4H), 5.56 (d, J=14.5 Hz, 2H), 5.49 (d, J=15.1 Hz, 4H), 5.38 (d, J=8.9 Hz, 2H), 5.27 (d, J=8.9 Hz, 2H), 5.18 (d, J=15.7 Hz, 4H), 4.09-4.04 (m, 10H), 1.68 (s, 6H), 1.62 (s, 6H). ¹³C NMR (DMSO-d₆, 125 MHz): δ 155.3, 154.2, 147.0, 126.1, 116.7, 77.4, 76.4, 70.7, 70.3, 48.1, 35.0, 17.1, 15.8 ppm (13 out of the 14 expected resonances were observed). HR-MS (ESI, positive, para-xylenediammonium dichloride as guest): m/z 551.22446 ([M+guest-2Cl⁻]²⁺), C₅₀H₅₈N₁₈O₁₂²⁺ calcd. for 551.22408.

Container 1. To a mixture of compound 2 (0.80 g, 0.83 mmol) and pyridine sulfur trioxide complex (2.6 g, 16.3 mmol) was added dry pyridine (25 mL). The resulting mixture was stirred at 90° C. under N₂ for 18 hours. The reaction mixture was cooled to RT. The product precipitated out of the solution and was collected by filtration. The solid was slurried in water (1 mL), and the pH was adjusted to 8.4 by slow addition of saturated aqueous NaHCO₃. After addition of EtOH (70 mL), the crude product was collected by centrifugation 7000 rpm×7 min. The precipitate was suspended in ethanol (50 mL×2), sonicated for 30 minutes, and solid collected by centrifugation. The crude solid was analyzed by NMR and process was repeated until the trapped pyridine was fully removed. Then the pH of the crude solution was adjusted to 7.0 by slow addition of 1 M HCl and the solvent was evaporated. The crude solid was treated with 30 mL of a mixture of CH₃CN/H₂O (2:1) and the heterogeneous mixture was centrifuged, the supernatant was collected and then evaporated to give a crude solid. The crude solid was redissolved in minimum amount of water and purified by size exclusion chromatography using Sephadex® G25 resin (30 mm×200 mm) and eluted by water. Pure product was collected as the front fractions with violet fluorescent color under UV long wavelength (366 nm). After drying under high vacuum, the compound 1 was obtained as a light yellow solid (0.50 g, 60% yield). M.p.>300° C., IR (ATR, cm⁻¹): 1720m, 1468s, 1228s, 1048s, 970m, 798s. ¹H NMR (D₂O, 500 MHz): δ 7.53 (s, 4H), 6.66 (d, J=15.4 Hz, 2H), 5.57 (d, J=15.8 Hz, 4H), 5.46 (d, J=8.9 Hz, 2H), 5.41 (d, J=8.9 Hz, 2H), 5.28 (d, J=16.5 Hz, 4H), 4.41 (d, J=16.5 Hz, 4H), 4.29 (d, J=15.8 Hz, 4H), 4.16 (d, J=15.4 Hz, 2H), 1.84 (s, 6H), 1.82 (s, 6H). ¹³C NMR (D₂O, 125 MHz, dioxane as external reference): δ 156.5, 155.9, 146.0, 131.9, 123.0, 78.3, 77.3, 71.1, 70.9, 52.5, 48.1, 35.8, 15.6, 14.6 ppm (14 out of the 14 expected resonances were observed). HR-MS (ESI, positive, 6d as guest): m/z 787.1679 ([M+6d-2Cl⁻]²⁺), C₅₄H₇₀N₁₈O₂₄Na₄²⁺ calcd. for 787.1642.

### Example 2

This example provides a description of in vivo activity of compounds of the present disclosure.

Cell Cytotoxicity Data for Container 1.

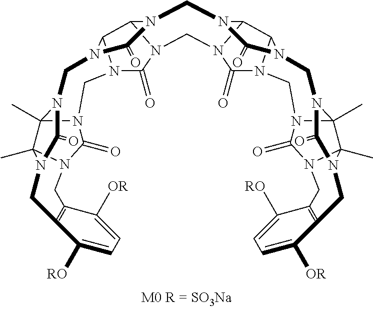

To test the Cytotoxicity and Cell Viability of the above compounds we used two different assays: an MTS (CellTiter 96 AQueous Kit®) assay that measures cellular metabolism, and the AK (Toxilight® BioAssay Kit) assay that measures cell death through release of the cytosolic enzyme adenylate kinase into the supernatant. Both assays were performed with two different cell lines. HEK293 and Hep G2 cells, are frequently used in drug toxicity studies. HEK293, a human kidney cell line, is used to evaluate the effect of the drug on the renal system and Hep G2, a human hepatocyte cell line, is used to assess the response of liver cells where drugs are metabolized. The MTS and AK assays for both cell lines were conducted after 24 h of incubation with the compounds at concentrations of 0.01 mM, 0.03 mM, 0.1 mM, 0.3 mM, and 1 mM. Eight technical replicates were designated for untreated cells and four technical replicates were designated for the cells treated with each compound and staurosporine (apoptosis inducer).

The collected absorbance and relative luminescence data were normalized to percent cell viability (MTS) and percent cell death (AK) using equations 1 and 2:

% cell viability=(Abs sample/Average Abs UT)×100 Eq. 1:

% cell death=(RLU samples/Average RLU Distilled water)×100 Eq. 2:

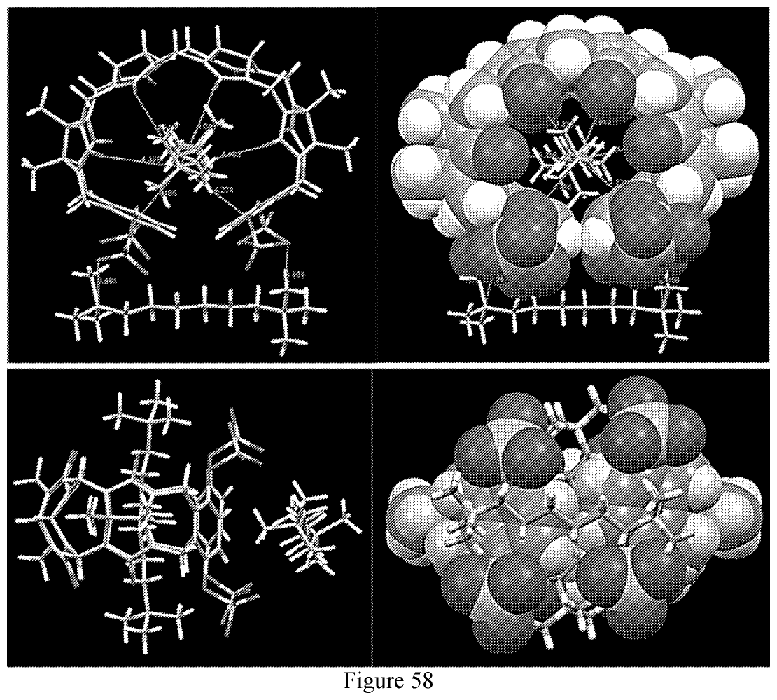

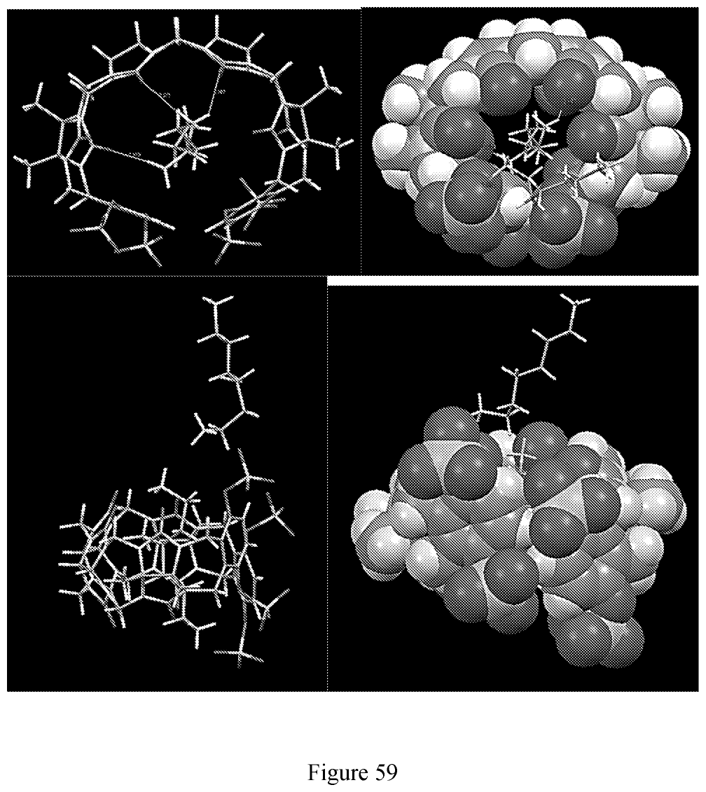

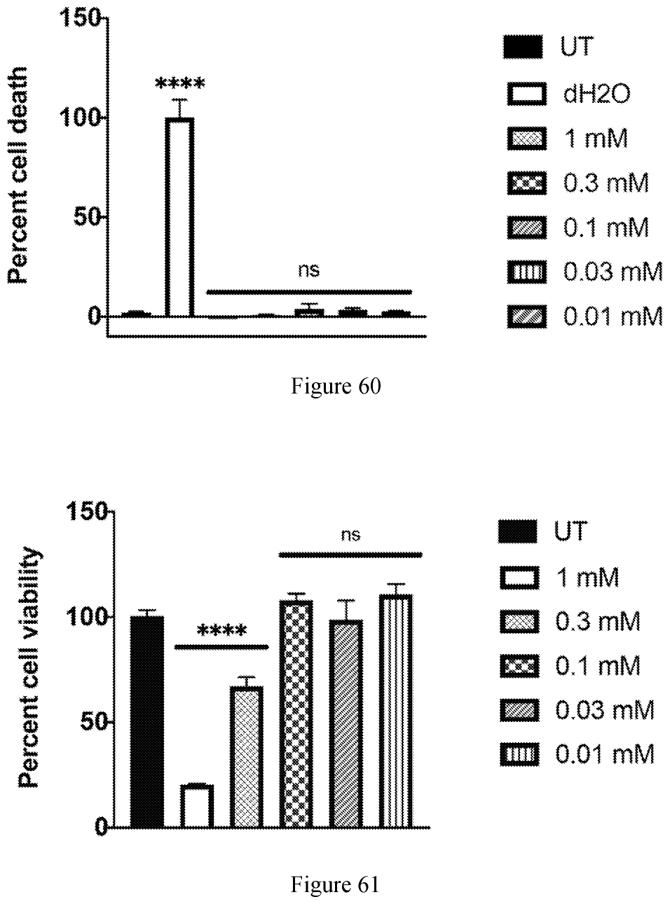

Toxicity studies using the MTS and AK assays for the liver cell line, HepG2 suggests that 1 demonstrates low cytotoxicity up to a concentration of 1 mM and high cell tolerance up to a concentration of 0.3 mM (FIGS. 60 and 61).

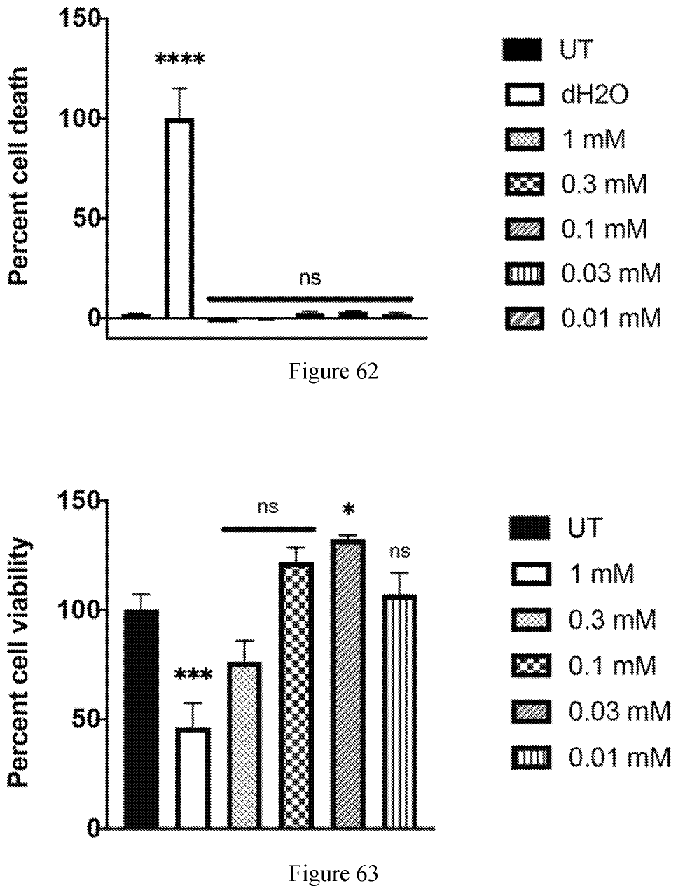

Similar toxicity studies performed on human kidney (HEK293) cells suggest that 1 demonstrates low cytotoxicity up to a concentration of 1 mM and high cell tolerance up to a concentration of 0.3 mM (FIGS. 62 and 63).

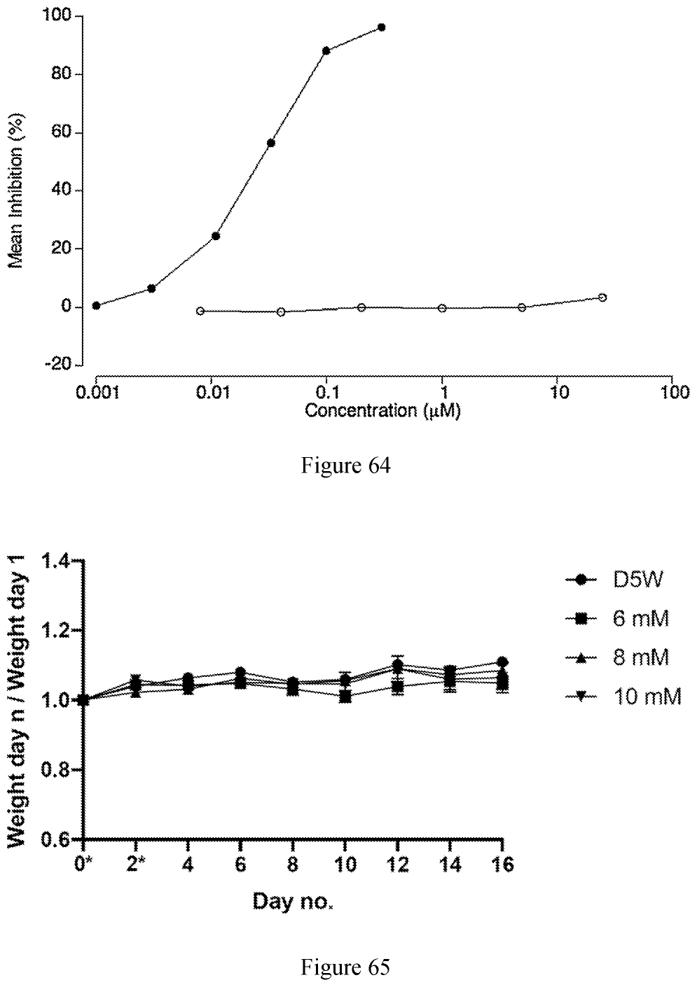

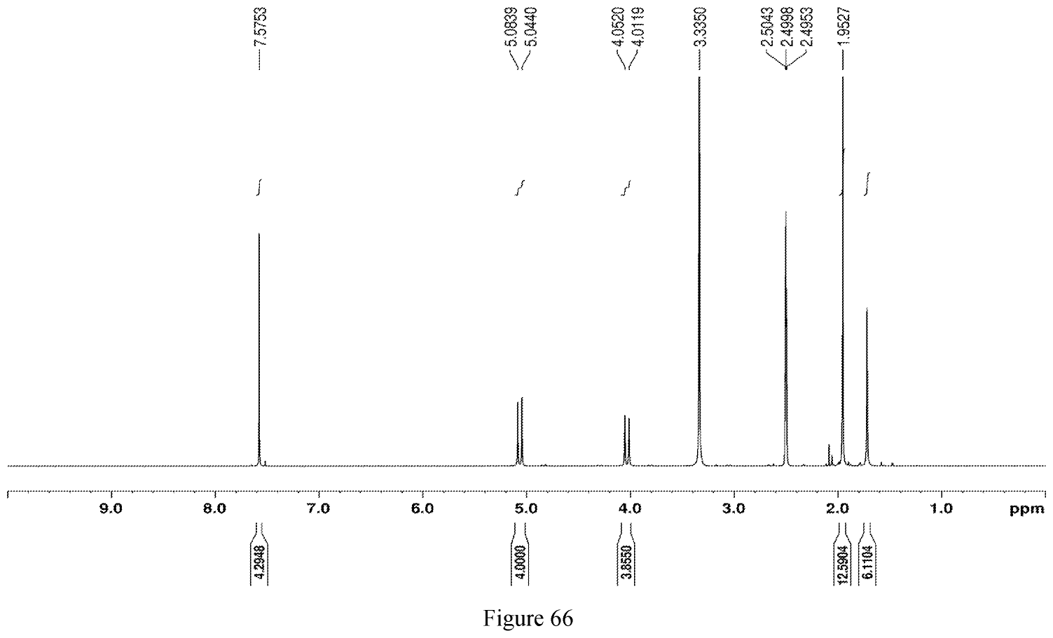

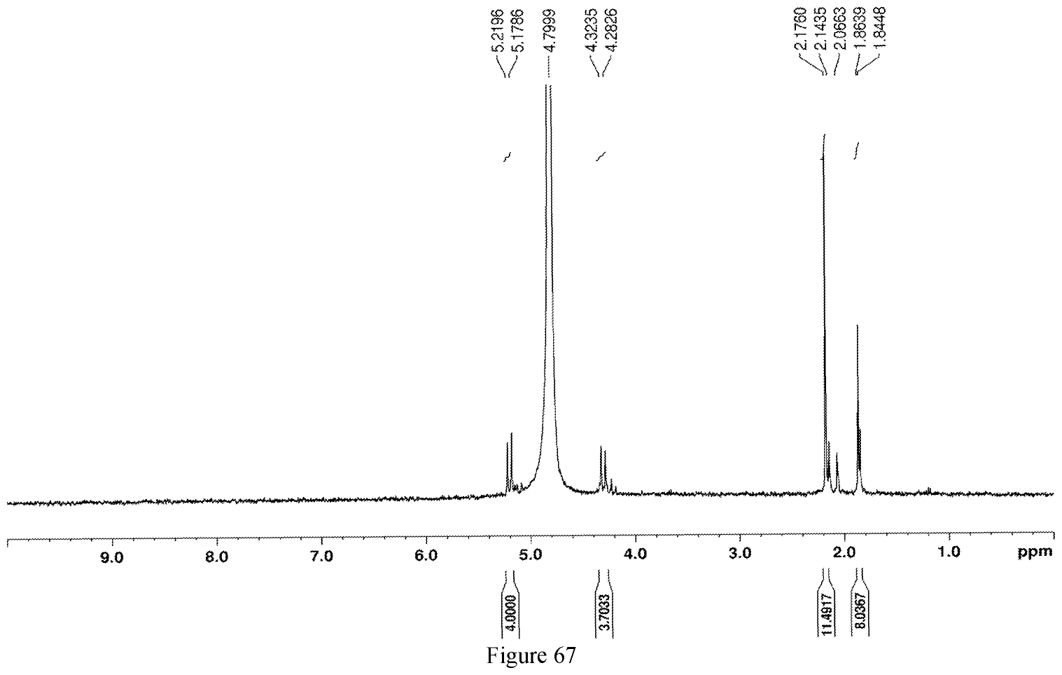

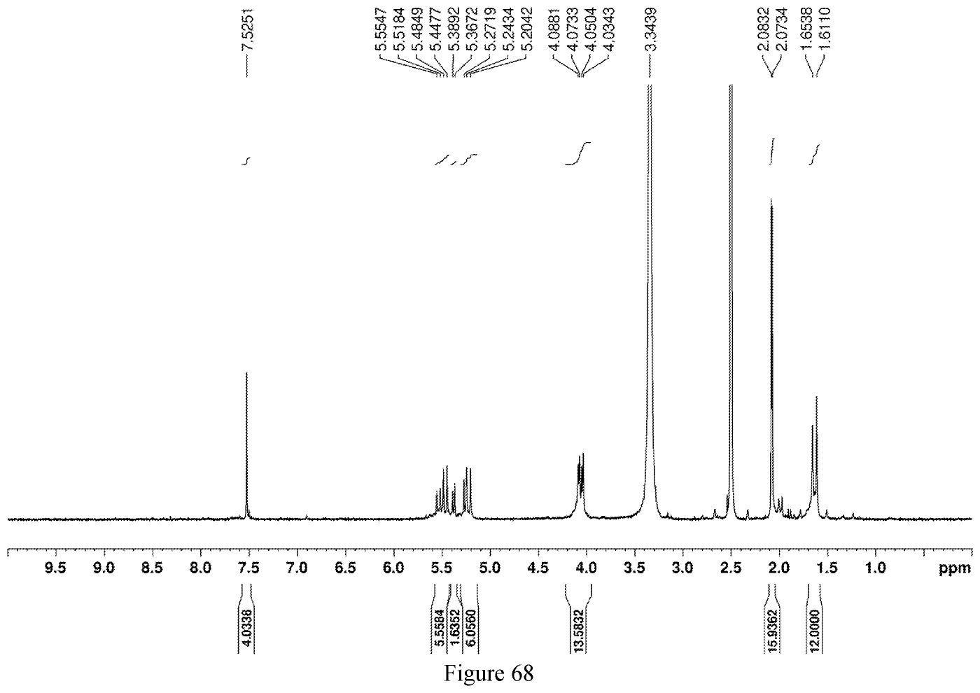

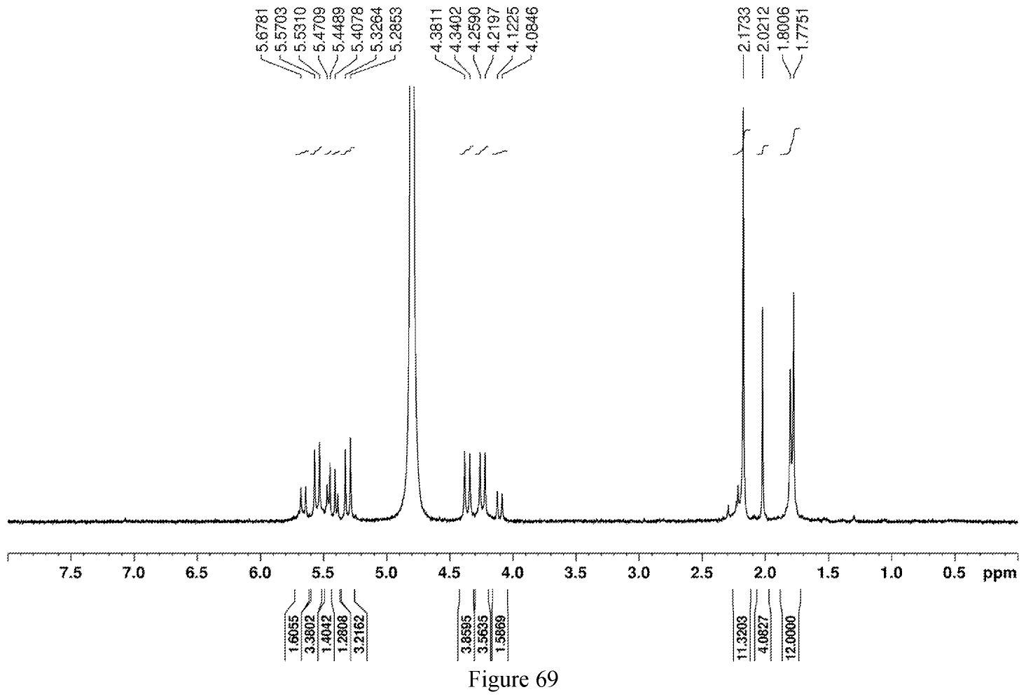

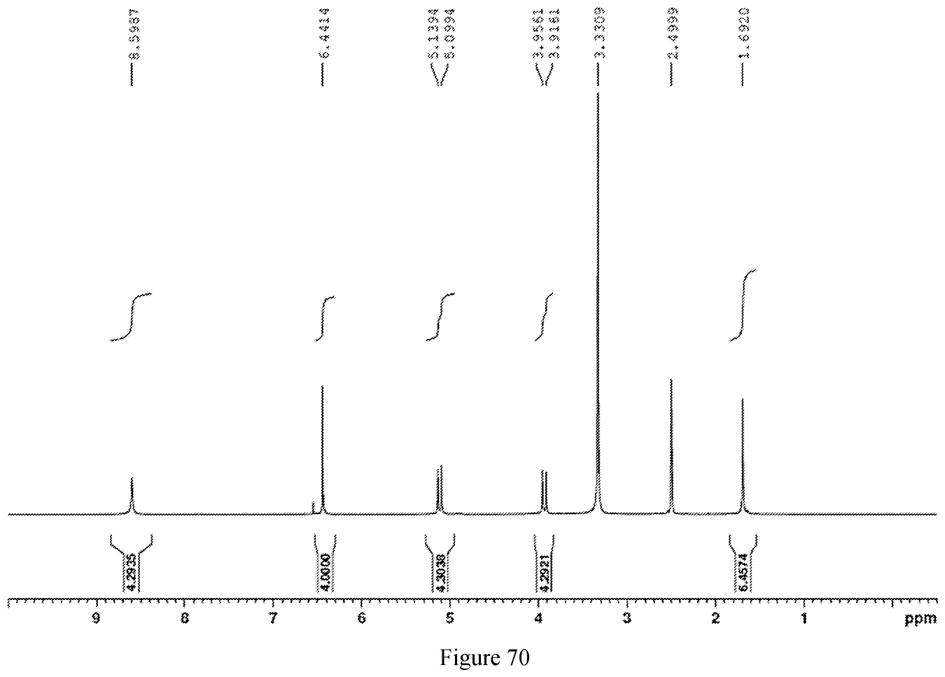

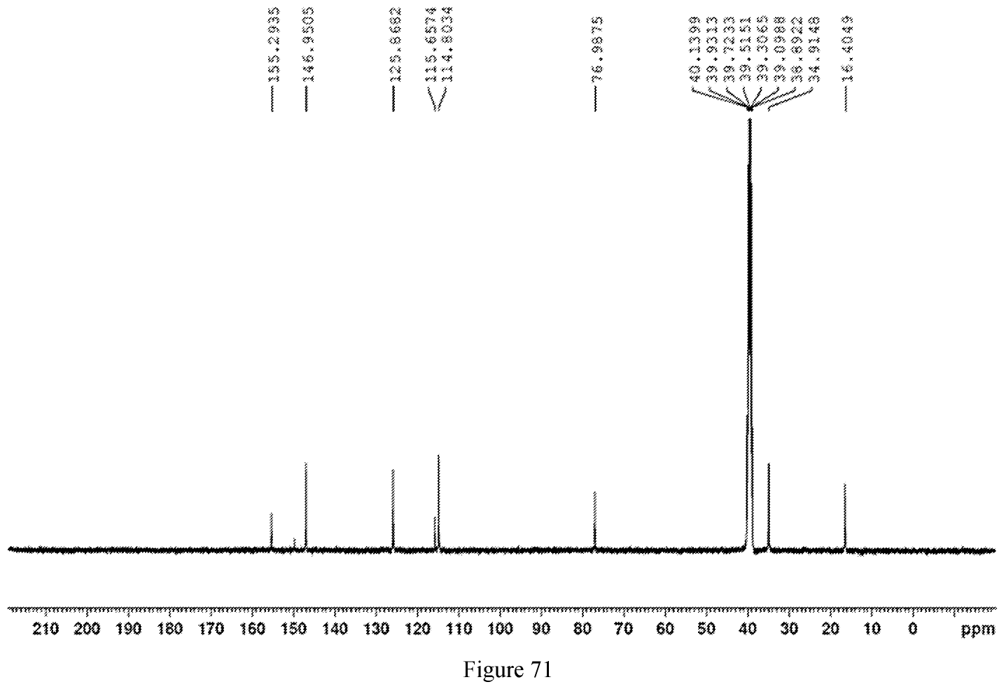

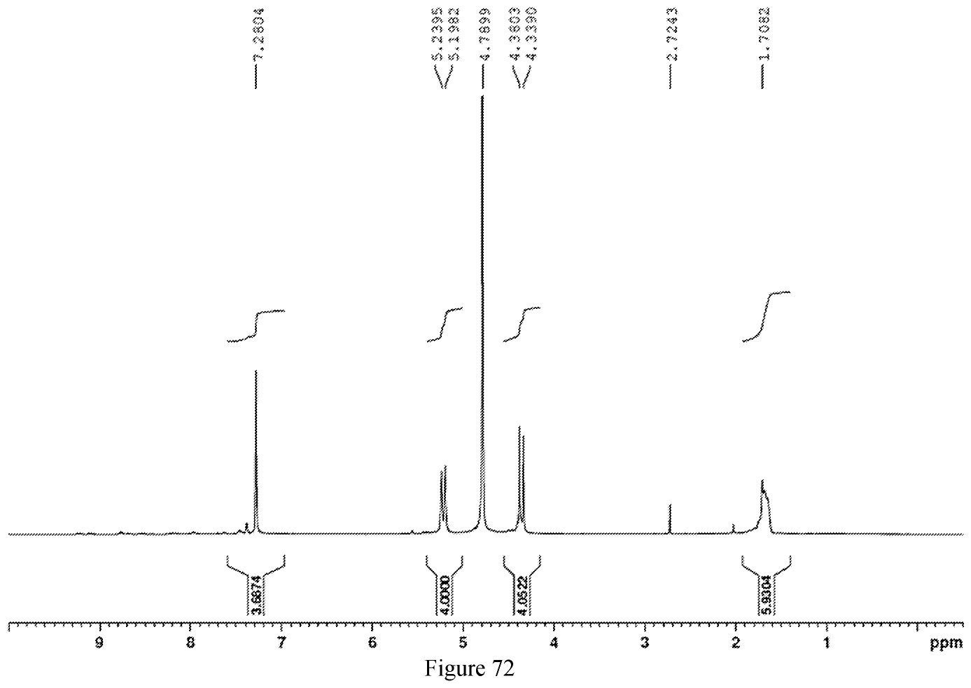

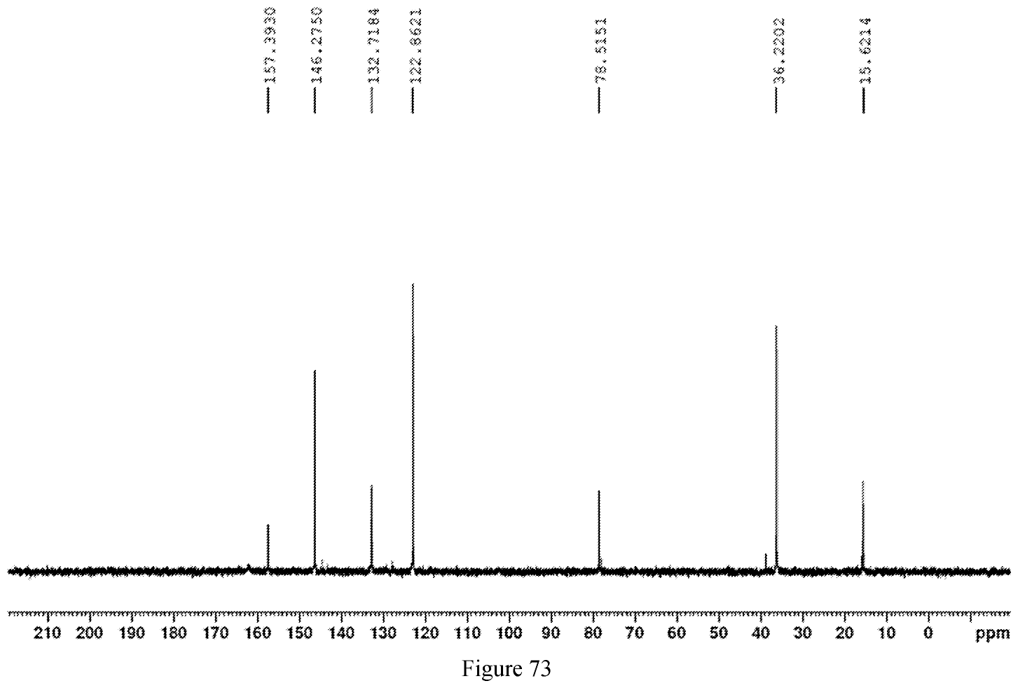

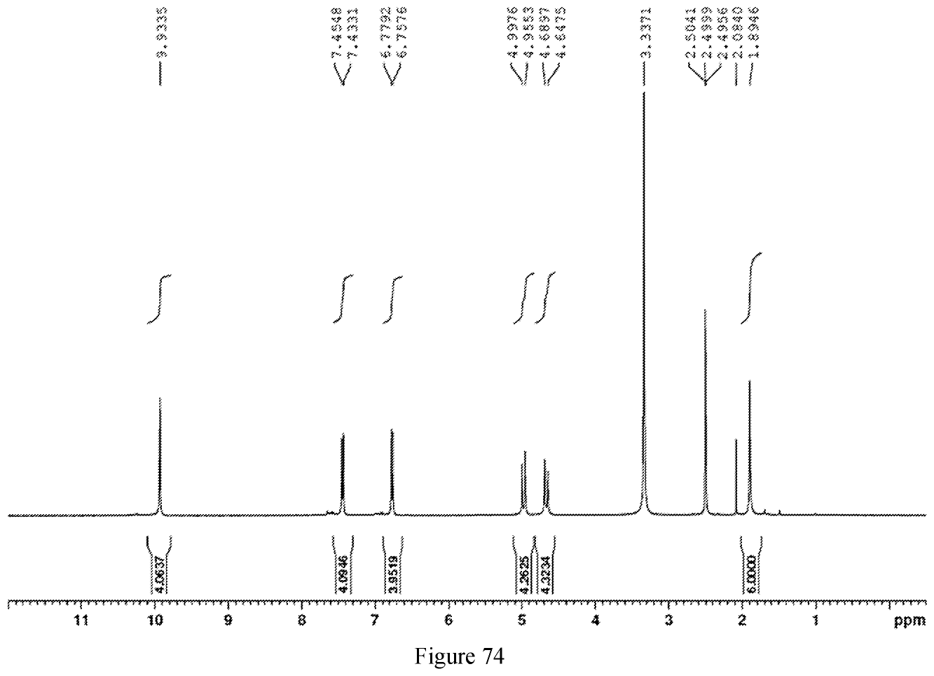

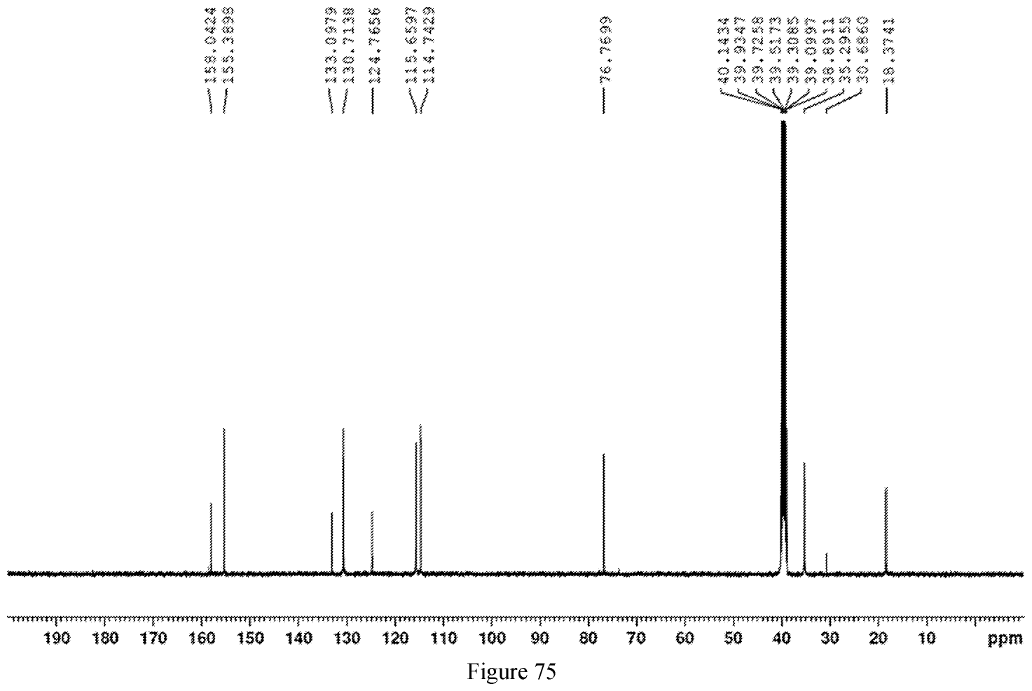

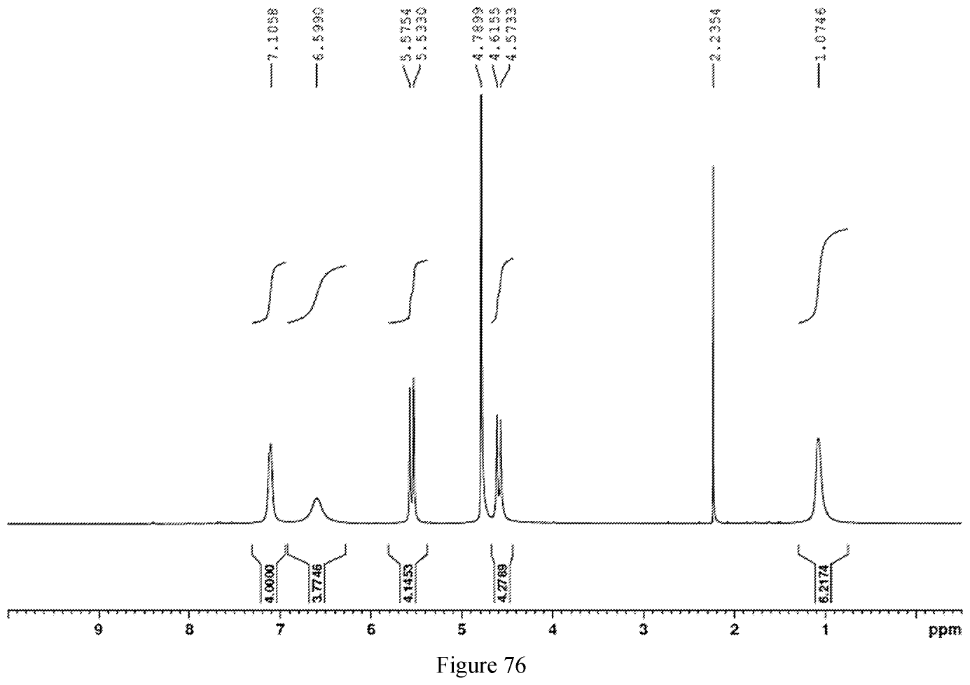

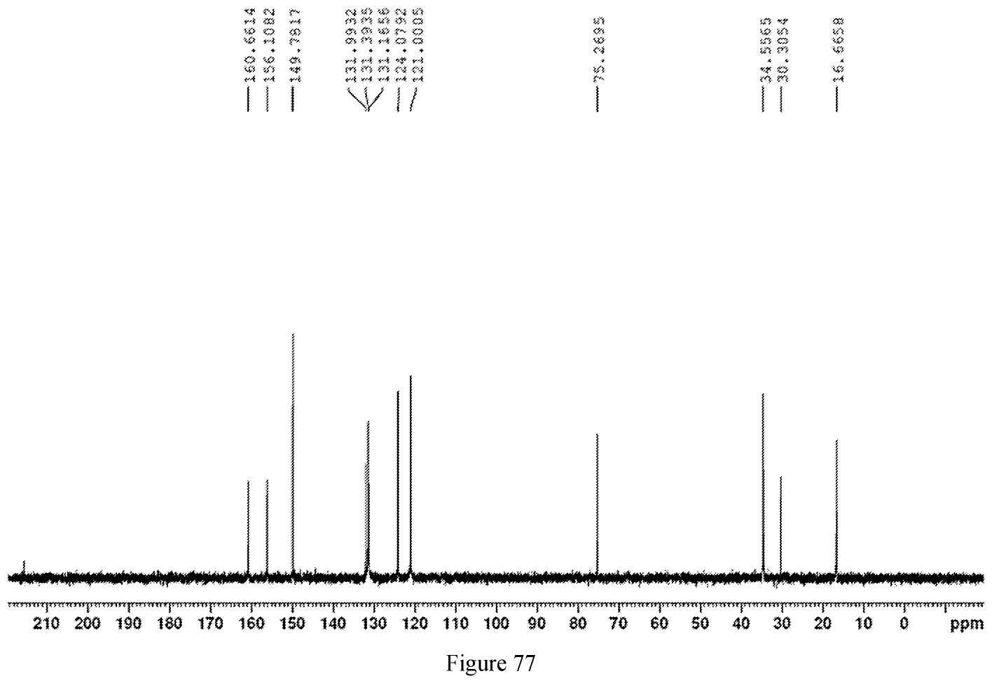

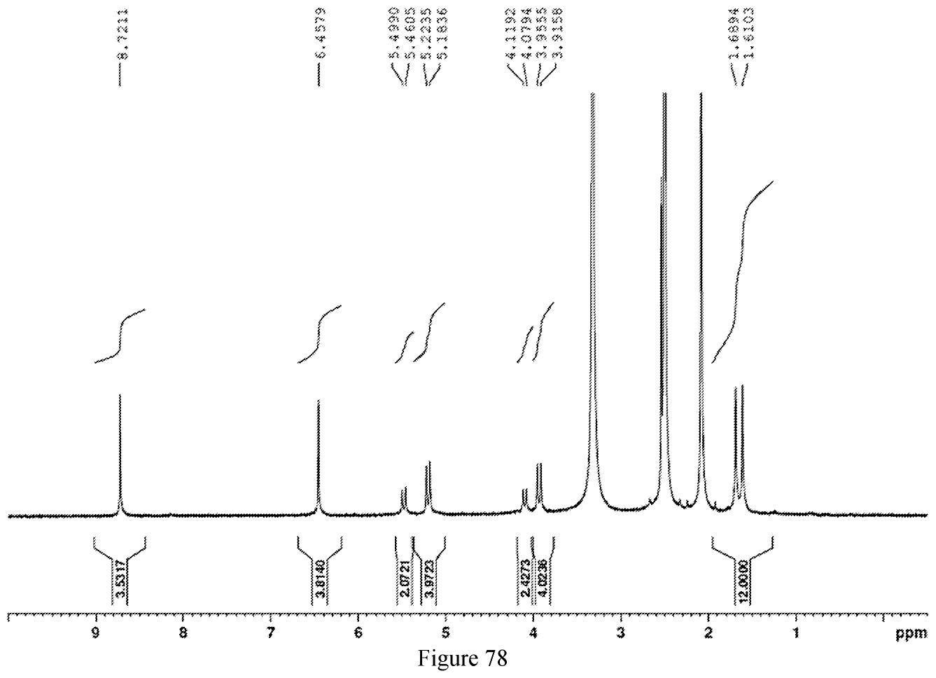

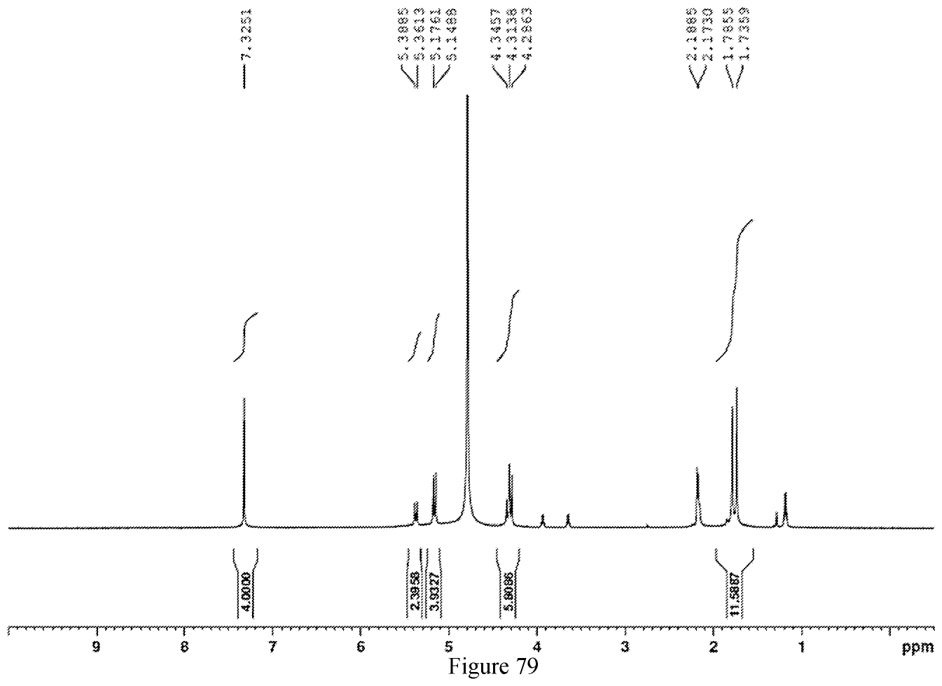

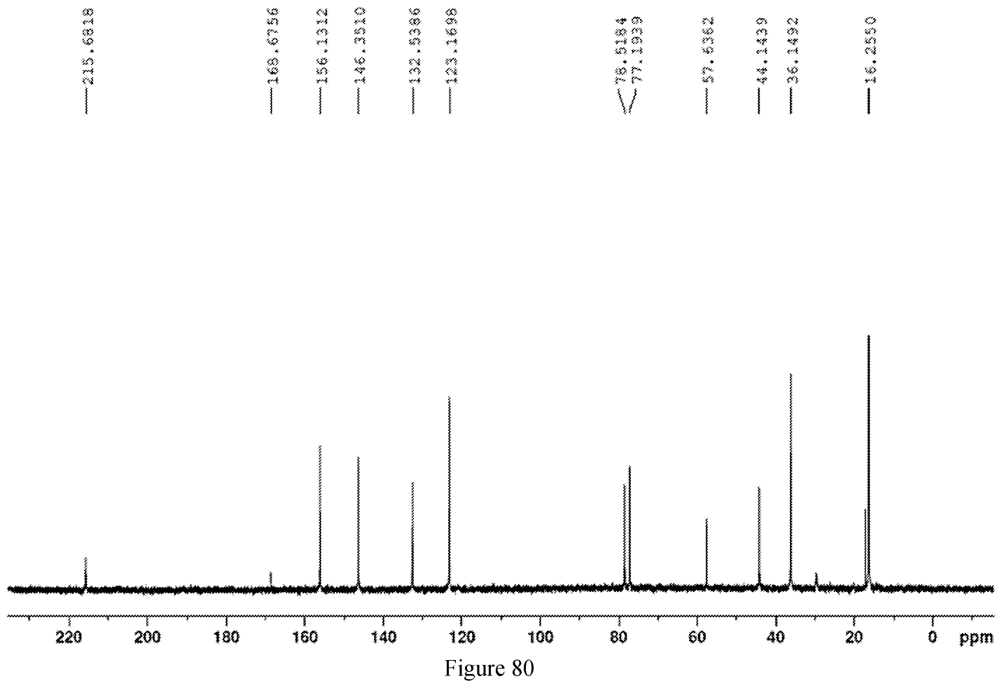

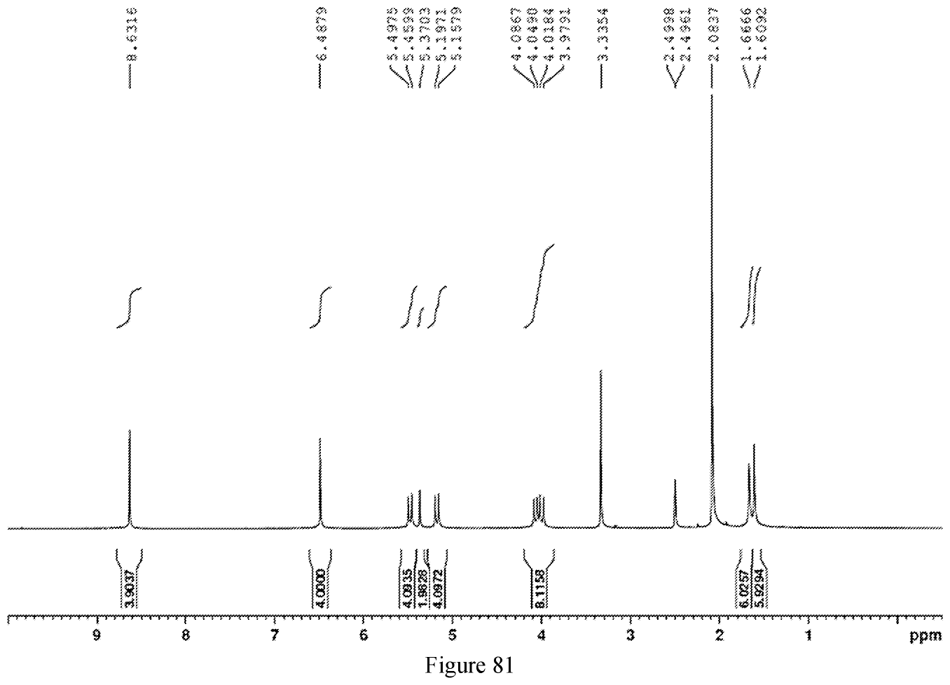

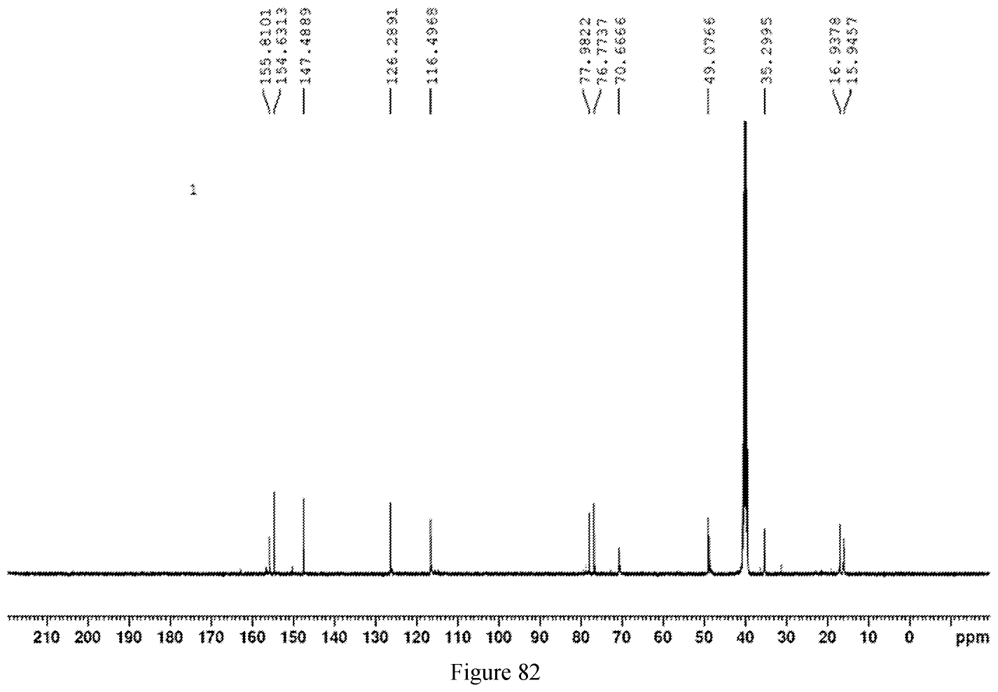

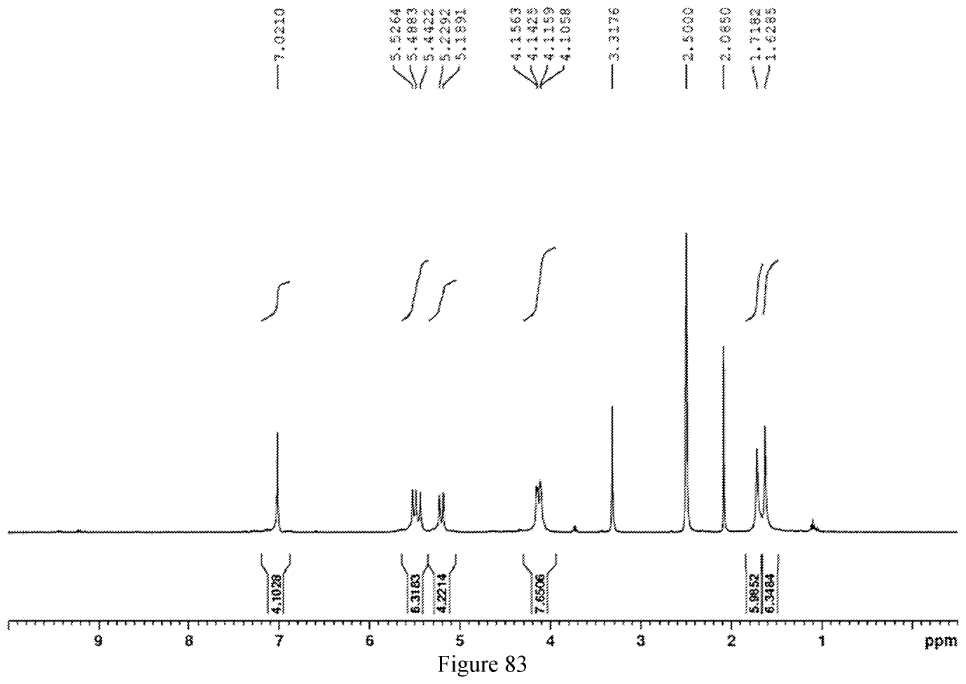

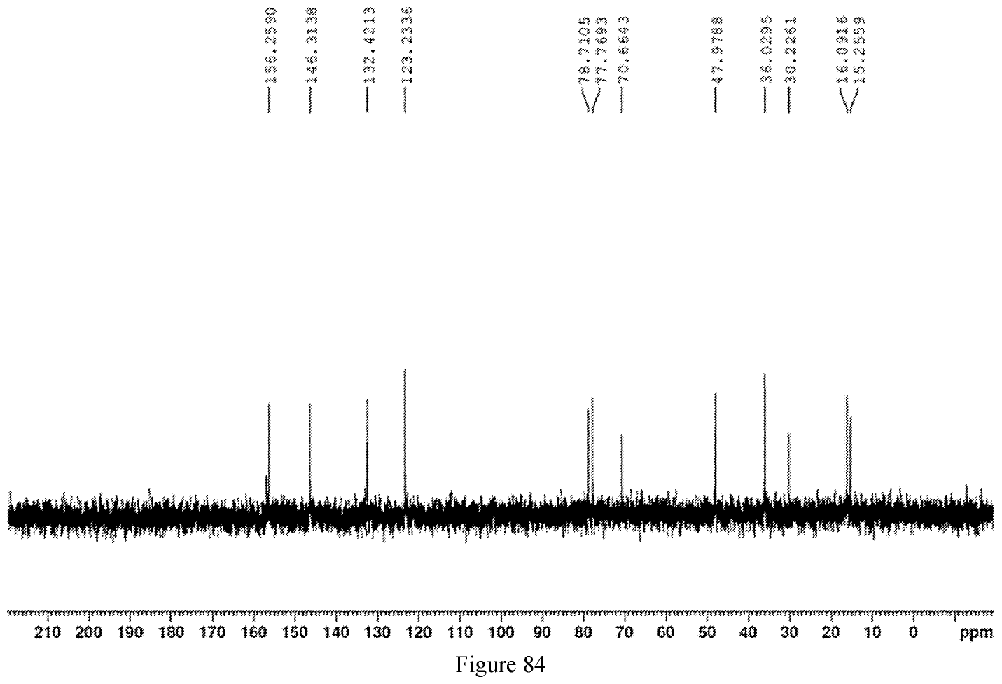

Human ether-a-go-go (hERG) Ion Channel Inhibition Assay. The hERG ion channel is a voltage-gated potassium channel in cardiac cells that is essential for cardiac repolarization. With the inhibition of this channel, the electrical depolarization and repolarization of the heart ventricles can be extended, leading to potentially fatal cardiac malfunction. The ability of 1 at six concentrations (0.008 μM to 25 μM) to inhibit the hERG ion channel function was evaluated by a contract research organization via the patch-clamp technique (QPatch HTX) following the general principles of whole-cell patch-clamping. The patch clamp hERG assay was conducted using mammalian cells (HEK-293) expressing the hERG channel. FIG. 64 shows the results of the hERG assay for 1 and for E-4031 as positive control. As can be readily seen, the positive control (E-4031) exhibits a sharp increase in inhibition of ion channel activity as the concentration increases past 0.1 μM. In contrast, no concentration dependent change in ion channel activity is observed for the cells treated with 1. The calculated IC₅₀ value for E-4031 is 0.3 μM whereas the IC₅₀ value for 1 is greater than 25 μM. IC₅₀ values below 0.1 μM are defined as highly potent inhibitors of the hERG channel, values between 0.1 and 1 μM as potent, values between 1-10 μM as moderately potent, and finally, IC₅₀ values above 10 μM are typically categorized as having little to no inhibition of the channel. Accordingly, 1 is not an inhibitor of the hERG ion channel which encourages the further development of the in vivo sequestering abilities of the compound.

In Vivo Maximum Tolerated Dose Study (MTD)

Animals studies were performed at the University of Maryland, Microbiology Building under the supervision of Dr. Volker Briken (IACUC #R-JAN-17-25). A total of 20 female Swiss Webster were used for this study. Three different concentrations of 1 (10 mM, 8.0 mM, 6.0 mM) dissolved in 5% aq. Dextrose (D5W) were used. A D5W control group was also included. Each concentration and control group contained 5 mice. The mice received the compound in 0.150 mL of D5W via tail vein injection, with 48 hours between injections. The weight and health status of the mice were monitored for 2 weeks following the last injection.

Synthesis of New Sulfated Acyclic CB[n] Type Receptors.

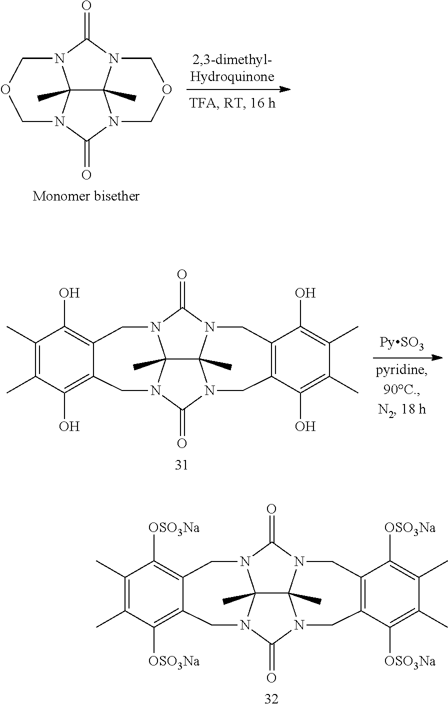

Compound 31. To a mixture of 2,3-dimethylhydroquinone (100 mg, 0.72 mmol) and glycoluril monomer (45 mg, 0.18 mmol) was added trifluoroacetic acid (2.45 ml). The mixture was stirred for 16 hours at RT under N₂. To the reaction mixture, MeOH (4.8 ml) was added and stirred for 1 hour at RT. The mixture was centrifuged (7000 rpm, 5 min) and the supernatant was removed. The solid pellet was washed with MeOH (3 mL), acetone (3 mL) and water (3 mL). In each of the washings the solid pellet was vortexed and sonicated with the respective solvent and finally centrifuged (7000 rpm, 5 min) and the supernatant decanted. The solid pellet was finally washed with acetone (3 mL) to remove traces of water. The residue was dried under the high vacuum to yield 31 as a white solid (35 mg, 39% yield). ¹H NMR (400 MHz, DMSO): δ 7.57 (s, 4H), 5.08 (d, 4H), 4.05 (d, 4H), 1.95 (s, 12H), 1.71 (s, 6H).

Compound 32. To a mixture of compound 31 (25 mg, 0.05 mmol), and pyridine sulfur trioxide (157 mg, 0.99 mmol) was added dry pyridine (1.5 mL). The resulting mixture was stirred for 18 hours at 90° C. under N₂. The mixture was cooled to RT. The formed precipitate was collected by decanting the solvent. The solid was slurried in water (0.1 mL) and the pH was adjusted to 8.4 by the slow addition of saturated aq. NaHCO₃. To the mixture, EtOH (4.26 ml) was added and the crude solid was collected by centrifugation (7000 rpm, 7 min) and the supernatant was decanted. The residue was suspended in EtOH (2×3 mL) and vortexed and sonicated until the solid pellet was in solution. The supernatant was collected and evaporated. To the resulting crude, EtOH was added until a precipitate is obtained. The precipitate was collected by centrifugation. The crude was analyzed by ¹H NMR to make sure all the pyridine has been removed. The crude was dissolved in water (2.5 mL) and acetone (15 mL) was added. The resulting mixture was kept at 4° C. overnight. The supernatant was collected and evaporated. The solid was dried under the high vacuum to yield 2. ¹H NMR (400 MHz, D₂O): δ 5.21 (d, 4H), 4.32 (d, 4H), 2.17 (s, 12H), 1.86 (s, 6H).

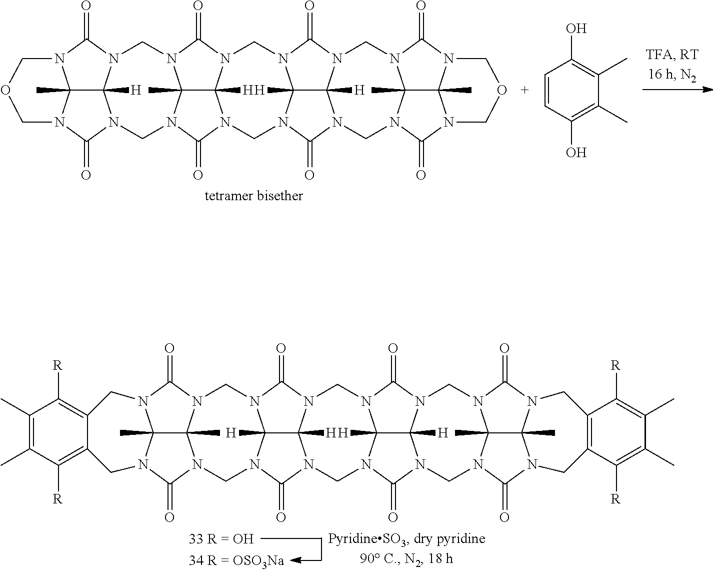

Compound 33. To a mixture of 2,3-dimethylhydroquinone (100 mg, 0.72 mmol) and tetramer bisether (141 mg, 0.18 mmol) was added trifluoroacetic acid (2.45 mL). The mixture was stirred for 16 hours at RT under N₂. To the reaction mixture, MeOH (5 mL) was added and stirred for 1 hour at RT. The mixture was centrifuged (7000 rpm, 5 min) and the supernatant was removed. The solid pellet was washed with MeOH (5 mL), acetone (3 mL), and water (5 mL), sequentially. In each of the washings the solid pellet was vortexed and sonicated with the respective solvent and finally centrifuged (7000 rpm, 5 min) and the supernatant decanted. The solid pellet was finally washed with acetone (5 mL) to remove traces of water. The residue was dried under high vacuum to yield 33 as a cream colored solid (80 mg, 43% yield). M.p>300° C. ¹H NMR (400 MHz, DMSO): δ 7.52 (s, 4H), 5.55-5.51 (m, 6H), 5.48 (d, 2H), 5.38-5.20 (m, 6H), 4.08-4.03 (m, 13H), 2.08 (s, 12H), 1.65 (s, 6H), 1.61 (s, 6H).

Compound 34. To a mixture of compound 33 (80 mg, 0.078 mmol), and pyridine sulfur trioxide (244.7 mg, 1.54 mmol) was added dry pyridine (2.4 ml). The resulting mixture was stirred for 18 hours at 90° C. under N₂. The mixture was cooled to RT. The formed precipitate was collected by decanting the solvent. The solid was slurried in water (0.1 ml) and the pH was adjusted to 8.4 by the slow addition of saturated NaHCO₃. To the mixture EtOH (16.4 ml) was added and the crude was collected by centrifugation (7000 rpm, 7 min). The residue was suspended in EtOH (10.8 ml×7) and vortexed and sonicated until the solid pellet was in solution. The crude was collected by centrifugation. The crude was analyzed by ¹H NMR to make sure all the pyridine has been removed. The pH of the crude solid was adjusted to 7 by the addition of 1 M HCl. The solvent was evaporated and 2:1 CH₃CN/H₂O (2.8 ml) was added to the solid. The mixture was centrifuged, and the supernatant was collected and evaporated to give a crude solid. The crude solid was dissolved in water (2.3 ml) and acetone (15 ml) was added. The precipitated was collected and recrystallized using water and acetone (solid was dissolved in 1.5 ml water while heating and acetone was added until the appearance of permanent turbidity). The resulting mixture was kept at 4° C. for a few minutes and the precipitate was collected by centrifugation. The residue was dried under the high vacuum to yield 34 as a white solid (107 mg, 95% yield). M.p>300° C. ¹H NMR (400 MHz, D₂O): δ 5.67 (d, 2H), 5.57 (d, 4H), 5.47 (d, 2H), 5.40 (d, 2H), 5.32 (d, 4H), 4.38 (d, 4H), 4.25 (d, 4H), 4.12 (d, 2H), 2.17 (s, 12H), 1.80 (s, 6H), 1.77 (s, 6H).

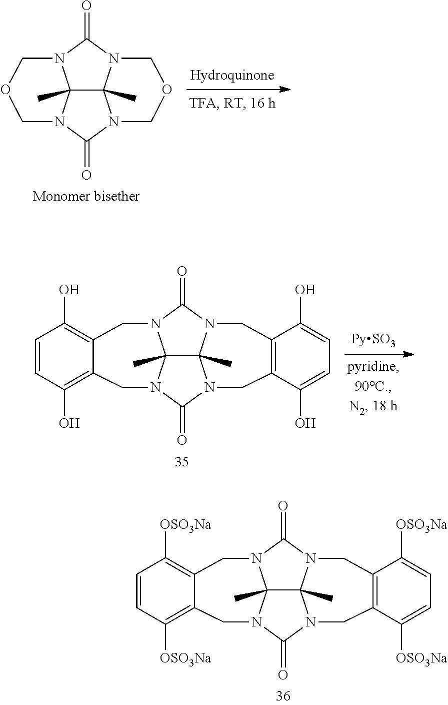

Compound 35: To a mixture of monomer bisether (0.10 g, 0.39 mmol) and hydroquinone (0.17 g, 1.56 mmol) were added trifluoroacetic acid (20 mL). The resulting heterogeneous mixture was stirred under N₂ at 25° C. for 16 hours. Then the reaction mixture was poured into MeOH (50 mL) and stirred for 1 hour. The crude product was collected by centrifugation and subsequently washed with MeOH (40 mL), acetone (40 mL×2) to remove the unreacted hydroquinone. The residue was dried under high vacuum to yield 35 as a pale yellow solid (0.15 g, yield 85%). ¹H NMR (DMSO-d₆, 400 MHz): δ 8.60 (s, 4H), 6.44 (s, 4H), 5.12 (d, J=16.0 Hz, 4H), 3.94 (d, J=16.0 Hz, 4H), 1.69 (s, 6H). ¹³C NMR (DMSO-d₆, 100 MHz): δ 155.3, 146.9, 125.8, 114.8, 76.9, 34.9, 16.4.

Compound 36: To a mixture of 35 (0.1 g, 0.23 mmol) and pyridine sulfur trioxide complex (0.73 g, 4.60 mmol) was added dry pyridine (15 mL). The resulting mixture was stirred at 90° C. under N₂ for 18 hours. The reaction mixture was cooled to RT. Then the product precipitated out of the solution and was collected by filtration. The solid was dissolved in water (4 mL), and the pH was adjusted to 8.4 by slow addition of saturated aqueous NaHCO₃. After addition of EtOH (40 mL), the crude product was collected by centrifugation 7000 rpm×7 min. The precipitate was suspended in EtOH (50 mL×2), sonicated for 30 minutes, and solid collected by centrifugation. The crude solid was analyzed by NMR and process was repeated until the trapped pyridine was fully removed. Then the pH of the crude solution was adjusted to 7.0 by slow addition of 1 M HCl and the solvent was evaporated. The crude solid was treated with 15 mL of a mixture of CH₃CN/H₂O (2:1) and the heterogeneous mixture was centrifuged, the supernatant was collected and then evaporated to give a crude solid. The crude solid was redissolved in minimum amount of water and purified by size exclusion chromatography using Sephadex® G25 resin (30 mm×200 mm) and eluted by water. After drying under high vacuum, the compound 36 was obtained as a yellow solid (0.11 g, 55% yield). ¹H NMR (D₂O, 400 MHz): δ 7.28 (s, 4H), 5.22 (d, J=16.5 Hz, 4H), 4.35 (d, J=16.5 Hz, 4H), 1.71 (s, 6H). ¹³C NMR (D₂O, 100 MHz): 157.4, 146.3, 132.7, 122.9, 78.5, 36.22, 15.6.

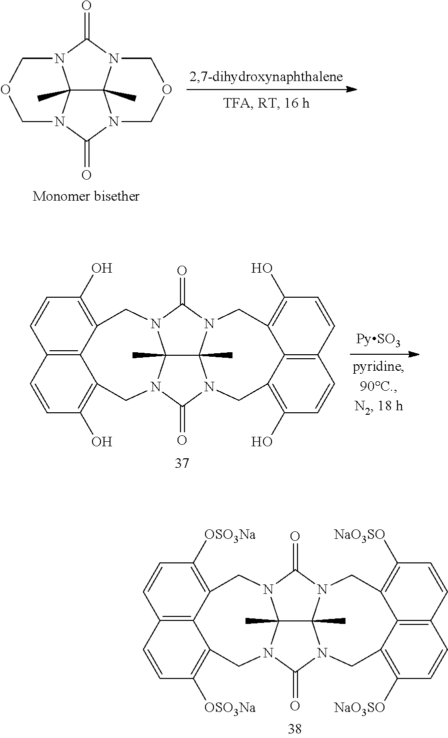

Compound 37: To a mixture of monomer bisether (0.10 g, 0.39 mmol) and 2,7-dihydroxynaphthalene (0.25 g, 1.56 mmol) were added trifluoroacetic acid (20 mL). The resulting heterogeneous mixture was stirred under N₂ at 25° C. for 16 hours. Then the reaction mixture was poured into MeOH (50 mL) and stirred for 1 hour. The crude product was collected by centrifugation and subsequently washed with MeOH (40 mL), acetone (40 mL×2) to remove the unreacted 2,7-dihydroxynaphthalene. The residue was dried under the high vacuum to yield 37 as a red solid (0.17 g, 80% yield). ¹H NMR (DMSO-d₆, 400 MHz): δ 9.33 (s, 4H), 7.44 (d, J=8.6 Hz, 4H), 6.77 (d, J=8.6 Hz, 4H), 4.98 (d, J=16.9 Hz, 4H), 4.67 (d, J=16.9 Hz, 4H), 1.89 (s, 6H). ¹³C NMR (DMSO-d₆, 100 MHz): δ 158.05, 155.39, 133.10, 130.72, 124.77, 115.66, 114.75, 76.77, 35.30, 18.38.

Compound 38: To a mixture of 37 (0.1 g, 0.19 mmol) and pyridine sulfur trioxide complex (0.60 g, 3.80 mmol) was added dry pyridine (15 mL). The resulting mixture was stirred at 90° C. under N₂ for 18 hours. The reaction mixture was cooled to RT. Then the product precipitated out of the solution and was collected by filtration. The solid was dissolved in water (4 mL), and the pH was adjusted to 8.4 by slow addition of saturated aqueous NaHCO₃. After addition of EtOH (40 mL), the crude product was collected by centrifugation 7000 rpm×7 min. The precipitate was suspended in EtOH (50 mL×2), sonicated for 30 minutes, and solid collected by centrifugation. The crude solid was analyzed by ¹H NMR and the process was repeated until the trapped pyridine was fully removed. Then the pH of the crude solution was adjusted to 7.0 by slow addition of 1 M HCl and the solvent was evaporated. The crude solid was treated with 15 mL of a mixture of CH₃CN/H₂O (2:1) and the heterogeneous mixture was centrifuged, the supernatant was collected and then evaporated to give a crude solid. The crude solid was redissolved in minimum amount of water and purified by size exclusion chromatography using Sephadex® G25 resin (30 mm×200 mm) and eluted by water. After drying under high vacuum, compound 38 was obtained as a light red solid (0.090 g, 50% yield). ¹H NMR (D₂O, 400 MHz): δ 7.10 (s, 4H), 6.59 (br., 4H), 5.55 (d, J=16.9 Hz, 4H), 4.59 (d, J=16.9 Hz, 4H) 1.07 (s, 6H). ¹³C NMR (D₂O, 100 MHz): 160.7, 156.1, 149.8, 132.0, 131.4, 131.2, 124.1, 121.0, 75.3, 34.6, 16.7.

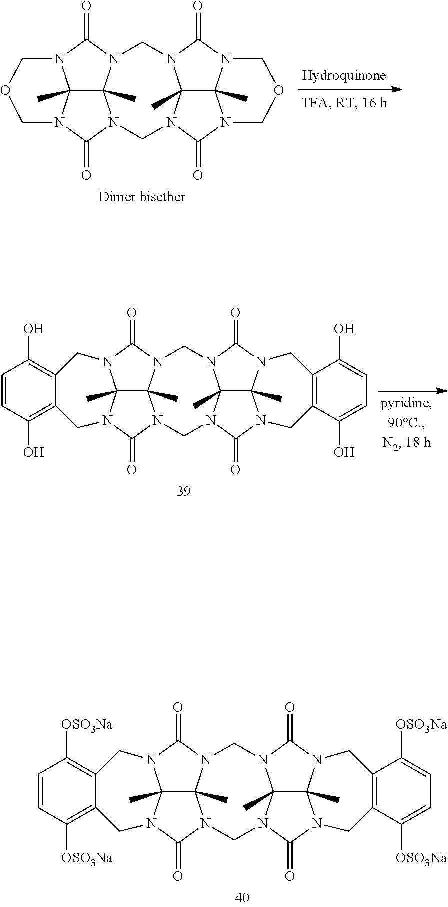

Compound 39. To a mixture of dimer bisether (0.10 g, 0.22 mmol) and hydroquinone (0.97 g, 0.88 mmol) were added trifluoroacetic acid (20 mL). The resulting heterogeneous mixture was stirred under N₂ at 25° C. for 16 hours. Then the reaction mixture was poured into MeOH (50 mL) and stirred for 1 hour. The crude product was collected by centrifugation and subsequently washed with MeOH (40 mL), acetone (40 mL×2) to remove the unreacted hydroquinone. The residue was dried under the high vacuum to yield 39 as a redish solid (0.12 g, yield 86%). ¹H NMR (DMSO-d₆, 400 MHz): δ 8.72 (s, 4H), 6.45 (s, 4H), 5.47 (d, J=15.5 Hz, 2H), 5.20 (d, J=16.0 Hz, 4H), 4.10 (d, J=15.5 Hz, 2H), 3.94 (d, J=16.0 Hz, 4H), 1.69 (s, 6H), 1.61 (s, 6H).

Compound 40. To a mixture of compound 39 (0.1 g, 0.16 mmol) and pyridine sulfur trioxide complex (0.51 g, 3.20 mmol) was added dry pyridine (15 mL). The resulting mixture was stirred at 90° C. under N₂ for 18 hours. The reaction mixture was cooled to RT. Then the product precipitated out of the solution and was collected by filtration. The solid was dissolved in water (4 mL), and the pH was adjusted to 8.4 by slow addition of saturated aqueous NaHCO₃. After addition of EtOH (40 mL), the crude product was collected by centrifugation 7000 rpm×7 min. The precipitate was suspended in EtOH (50 mL×2), sonicated for 30 minutes, and solid collected by centrifugation. The crude solid was analyzed by ¹H NMR and process was repeated until the trapped pyridine was fully removed. Then the pH of the crude solution was adjusted to 7.0 by slow addition of 1 M HCl and the solvent was evaporated. The crude solid was treated with 15 mL of a mixture of CH₃CN/H₂O (2:1) and the heterogeneous mixture was centrifuged, the supernatant was collected and then evaporated to give a crude solid. The crude solid was redissolved in minimum amount of water and purified by size exclusion chromatography using Sephadex® G25 resin (30 mm×200 mm) and eluted by water. After drying under high vacuum, compound 40 was obtained as a yellow solid (0.99 g, 60% yield). ¹H NMR (D₂O, 600 MHz): δ 7.32 (s, 4H), 5.37 (d, J=16.0 Hz, 2H), 5.16 (d, J=16.0 Hz, 4H), 5.16 (d, J=16.0 Hz, 4H), 4.35-4.28 (m, 6H), 1.79 (s, 6H), 1.74 (s, 6H). ¹³C NMR (D₂O, 150 MHz): 215.7, 156.1, 146.4, 132.5, 123.2, 78.5, 77.2, 57.6, 44.1, 36.1, 17.1, 16.3.

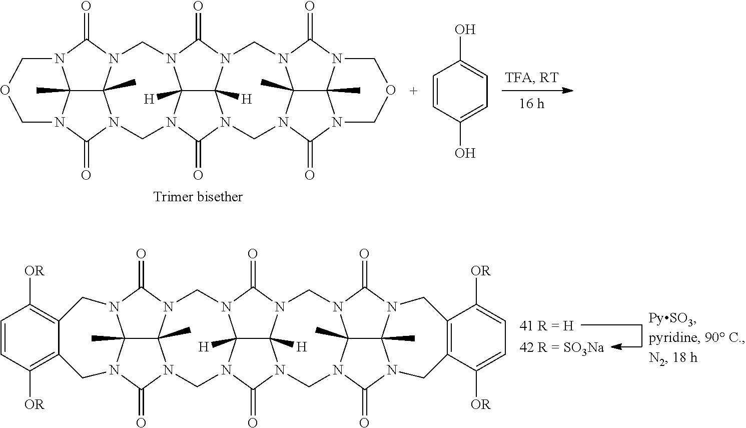

Compound 41. To a mixture of trimer bisether (0.10 g, 0.16 mmol) and hydroquinone (0.70 g, 0.64 mmol) were added trifluoroacetic acid (10 mL). The resulting heterogeneous mixture was stirred under N₂ at 25° C. for 16 hours. Then the reaction mixture was poured into MeOH (50 mL) and stirred for 1 hour. The crude product was collected by centrifugation and subsequently washed with MeOH (40 mL), acetone (40 mL×2) to remove the unreacted hydroquinone. The residue was dried under the high vacuum to yield 41 as a red solid (0.15 g, yield 80%). ¹H NMR (DMSO-d₆, 400 MHz): δ 8.63 (s, 4H), 6.49 (s, 4H), 5.48 (d, J=15.0 Hz, 4H), 5.37 (s, 2H), 5.18 (d, J=15.70 Hz, 4H), 4.03 (q, J=14.35 Hz, 8H), 1.67 (3, 6H), 1.60 (3, 6H). ¹³C NMR (DMSO-d₆, 100 MHz): δ 155.81, 154.63, 126.29, 116.50, 77.98, 76.77, 70.66, 49.08, 35.30, 16.94, 15.94.

Compound 42. To a mixture of compound 41 (0.1 g, 0.12 mmol) and pyridine sulfur trioxide complex (0.38 g, 2.40 mmol) was added dry pyridine (15 mL). The resulting mixture was stirred at 90° C. under N₂ for 18 hours. The reaction mixture was cooled to RT, the product precipitated out of the solution and was collected by filtration. The solid was dissolved in water (4 mL), and the pH was adjusted to 8.4 by slow addition of saturated aqueous NaHCO₃. After addition of EtOH (40 mL), the crude product was collected by centrifugation 7000 rpm×7 min. The precipitate was suspended in EtOH (50 mL×2), sonicated for 30 minutes, and the solid collected by centrifugation. The crude solid was analyzed by NMR and process was repeated until pyridine was fully removed. Then the pH of the crude solution was adjusted to 7.0 by slow addition of 1 M HCl and the solvent was evaporated. The crude solid was treated with 15 mL of a mixture of CH₃CN/H₂O (2:1) and the heterogeneous mixture was centrifuged, the supernatant was collected and then evaporated to give a crude solid. The crude solid was redissolved in minimum amount of water and purified by size exclusion chromatography using Sephadex® G25 resin (30 mm×200 mm) using water as the eluent. After drying under high vacuum, compound 42 was obtained as a yellow solid (0.79 g, 55% yield). ¹H NMR (DMSO-d₆, 400 MHz): δ 7.02 (s, 4H), 5.48 (t, J=16.8 Hz, 6H), 5.16 (d, J=16.0 Hz, 4H), 4.13 (d, J=6.73 Hz, 8H), 1.72 (s, 6H), 1.63 (s, 6H). ¹³C NMR (D₂O, 100 MHz): 156.3, 146.3, 132.4, 123.2, 78.7, 77.8, 70.7, 47.9, 36.0, 30.2, 16.1, 15.3.

In Vivo Reversal of Methamphetamine Induced Hyperlocomotion by 1 (which may be referred to as M0 or Motor0.)

Animals

Fifteen male Swiss Webster (CFW) mice were obtained from Charles River Laboratories that weighed ˜30 g upon arrival. Mice were individually housed in a temperature- and humidity-controlled room on a 12 h light/dark schedule with lights on at 6:00 am EST. For the duration of both experiments mice had ad libitum access to food and water. All behavioral testing occurred between 6:30 am and 2:00 pm EST, and all experimental procedures were approved by the University of Maryland Animal Care and Use Committee and conformed to the guidelines set forth by the National Research Council.

Surgical Procedures

Mice were anesthetized with an intraperitoneal (IP) injection of ketamine (100 mg/kg)/xylazine (10 mg/kg) (n=8) and were implanted with jugular catheters with head-mounted ports. All surgical procedures were conducted using aseptic technique, with body temperature monitored and maintained throughout surgery. Catheters were placed in the right jugular vein with the port passed subcutaneously out towards the top of skull. Ports (5 MM Up Pedestal; P1 Technologies) were fixed to the skull with a combination of super glue (Loctite) and dental cement. Following surgery, mice received an immediate injection of Rimadyl (5 mg/kg) and 0.4 mL of warm sterile saline. Mice were treated post-operatively for two days with Rimadyl (5 mg/kg) and given a minimum of 5 days to recover before resuming training. Catheters were flushed daily with 0.1 mL sterile saline solution containing gentamycin (0.33 mg/mL) and 0.1 mL sterile saline solution containing heparin (20 IU/mL) in order to reduce clotting and maintain catheter patency. Catheter patency was assessed daily from the first day following surgery until the end of testing. Any mouse whose catheter exhibited significant flowback on a majority of days was excluded from analysis.

Behavioral Testing

Mice were trained on a standard autoshaping task described previously. All behavioral procedures were conducted in a Med Associates test chamber equipped with a food cup, a retractable lever, and 4 floor IR photobeams. Time stamps were generated from head entries into the food cup, downward deflections of the lever, or disruption of floor beams and recorded by the behavioral computer.

In order to minimize the impact of novelty-induced suppression of feeding, mice were given five to six 20 mg sucrose pellets (Bioserv) each in their home cage for 2-3 days prior to the beginning of training. Mice were weighted and handled daily upon arrival until the completion of testing.

Following surgery, mice were habituated to the behavioral box and underwent one session of autoshaping to establish baseline locomotion levels before treatment began. Pavlovian training sessions which consisted of the presentation of the lever (CS) for 8 s, which was immediately followed by the delivery of a sucrose pellet and the retraction of the lever. The CS was presented on a random interval of 90±30 s schedule. Each Pavlovian session consisted of 30 trials. In total baseline plus testing lasted 9 consecutive days.

Experimental Design

Motor0 efficacy was assessed using a semi-counterbalanced design where all mice received each possible experimental treatment. The purpose of the experiments was to: (1) verify that binding of methamphetamine by Motor0 would not be compromised in vivo, (2) verify that Motor0 would not alter locomotor behavior, and (3) to demonstrate that Motor0 can sequester methamphetamine in vivo. On the first day of testing, regardless of experiment, mice underwent a baseline session free of treatment. On the following six sessions mice were treated with one of six possible treatments: 5% aqueous dextrose (D5W, 0.2 mL infused), Motor0 only (6 mM in D5W; 0.178 mL infused), methamphetamine only (4.24 mM in D5W; 0.5 mg/kg; 0.022 mL infused), a premixed solution of Motor0 and methamphetamine (Premix; ˜11.6:1 Motor0:Meth; 0.178 mL Motor0+0.022 mL Meth infused), Motor0 followed by methamphetamine administered 30 s later (0.178 mL of 6 mM Motor0 in D5W, 0.022 mL Meth infused), and methamphetamine followed by Motor0 administered 30 s later (0.022 mL of 4.24 mM Meth in D5W, 0.178 mL of 6 mM Motor0 in D5W infused). Mice received only one treatment per day. The dose of methamphetamine was chosen based on previously published values that observed reliable hyperlocomotion in mice. The smallest dosage that reliably induced hyperlocomotion was chosen.

Following completion of the first six sessions, mice completed another two days of behavioral testing. On day 8, half of the mice (n=8) received methamphetamine followed by Motor0 administered 5 minutes later (0.022 mL of 4.24 mM Meth in D5W infused, 0.178 mL 6 mM Motor0 in D5W), followed by infusion of methamphetamine followed by D5W administered 5 minutes later (0.022 mL of 4.24 mM Meth in D5W, 0.178 mL D5W infused) administered on the ninth day of testing. The other half of the mice (n=7) received the same exact treatment but in reverse order across days 8 and 9.

For each experiment, total locomotion counts (i.e., the total number of beam breaks) were obtained for each mouse across the entirety of each training session. For each experiment, locomotion counts were then analyzed across treatments using one-way repeated measures ANOVAs with tukey-corrected pairwise post-hoc t-tests in Graphpad Prism (Version 9.0.0).

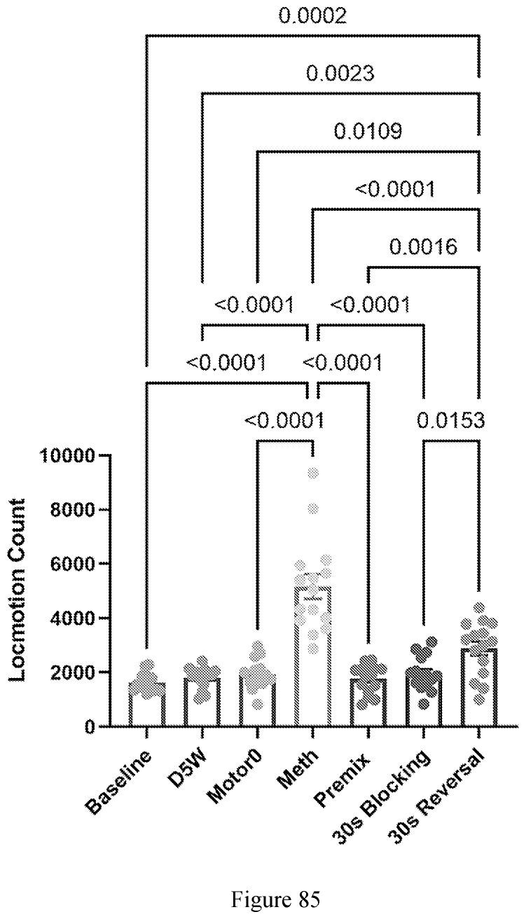

FIG. 85 shows in vivo reversal of methamphetamine-induced hyperlocomotion by Motor0. Average locomotion counts for male Swiss Webster mice (n=15; avg weight (g)±SD: 33.27±1.44) are plotted as a function of treatment. All mice underwent an initial habituation to determine baseline locomotion levels before treatment. Following this baseline measure, treatment order was counterbalanced across days, and mice only received one treatment per day. Over six consecutive days of testing mice each received a single treatment of 5% aqueous dextrose (D5W; 0.2 mL infused), Motor0 only (Motor0; 6 mM in D5W; 0.178 mL infused), methamphetamine only (4.24 mM Meth in D5W; 0.5 mg/kg; 0.022 mL infused), a premixed solution of Motor0 and methamphetamine (Premix; ˜11.6:1 Motor0:Meth; 0.2 mL infused), Motor0 followed by methamphetamine administered 30 s later (30s Blocking; 0.178 mL of 6 mM Motor0 in D5W, 0.022 mL of 4.24 mM Meth in D5W infused), and methamphetamine followed by Motor0 administered 30 s later (30 s Reversal; 0.022 mL of 4.24 mM Meth in D5W, 0.178 mL of 6 mM Motor0 in D5W infused). Bars represent average locomotion counts. Error bars represent the standard error of the mean (SEM). Dots represent counts for each mouse (n=15). Presented p-values are only for significant (p<0.05) Tukey-corrected post-hoc comparisons.

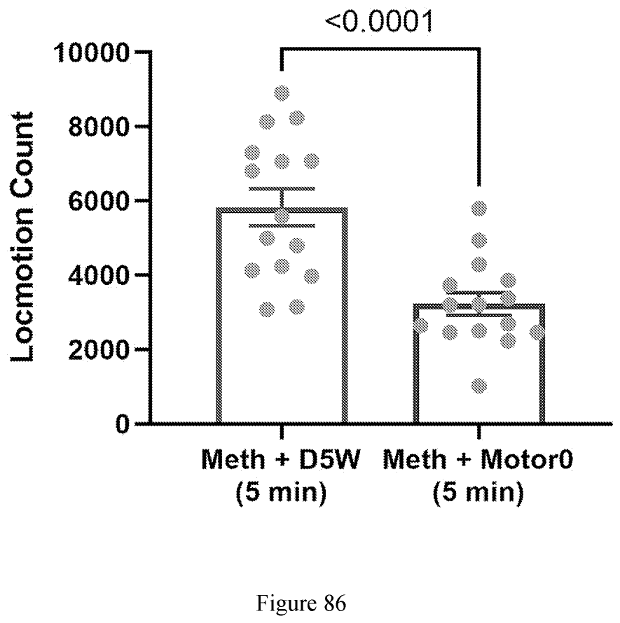

FIG. 86 shows in vivo reversal of methamphetamine-induced hyperlocomotion by Motor0 following 5-minute inter-injection interval. Average locomotion counts for male Swiss Webster mice (n=15; avg weight (g)±SD: 33.27±1.44) are plotted as a function of treatment. Mice receive either methamphetamine (4.24 mM Meth in D5W; 0.5 mg/kg; 0.022 mL infused) followed by D5W (0.178 mL infused) or Motor0 (6 mM in D5W; 0.178 mL infused) administered 5 minutes apart before being placed into the behavioral box. Bars represent average locomotion counts. Error bars represent the standard error of the mean (SEM). Dots represent counts for each mouse (n=15). Data analyzed using a paired t-test.

Discussion

The efficacy of Motor0 in the sequestration of methamphetamine was investigated in vivo. Fifteen male Swiss Webster (CFW) mice were trained on an Pavlovian autoshaping task described previously and locomotion values were obtained and analyzed accordingly. To establish methamphetamine induced hyperlocomotion and examine the in vivo efficacy of Motor0, mice were first treated with single infusions of D5W, Motor0 only, methamphetamine only, a premixed solution of Motor0 and methamphetamine, Motor0 followed by methamphetamine administration 30s later, or methamphetamine followed by Motor0 administered 30s later in counterbalanced manner. FIG. 85 depicts the results of this experiment by plotting locomotion counts as a function of treatment. Mixed effects analysis revealed a significant main effect of treatment (F(6,84)=44.43, p=0.0001) with Tukey-corrected post-hoc comparison showing a significant increase in locomotion counts for treatment with methamphetamine against all other treatments (p's<0.05). From these results it is clear that Motor0 alone does not significantly change the locomotion levels of the animals, but that the premix and blocking experiments effectively reduce the locomotion levels to that observed for D5W alone which establishes that they sequester Meth in vivo. The locomotion levels observed for the reversal experiment (e.g., Meth then Motor0 30 seconds later) are somewhat higher than that observed for the Blocking experiment but still significantly lower than Meth alone.

Although the results of this first analysis are suggestive of the potential efficacy of Motor0 in the sequestration of methamphetamine and inducing behavioral change, it is possible that the 30s interval between methamphetamine administration and Motor0 administration in the reversal condition is too short to be ethologically relevant. To address this issue, a follow up experiment was conducted where on days 8 and 9 of testing, mice (n=15) were administered either methamphetamine followed by administration of D5W 5 minutes later or methamphetamine followed by Motor0 (6 mM in D5W) 5 minutes later in a counterbalanced manner before completing the autoshaping task. FIG. 86 plots locomotion counts as a function of either treatment. A significant decrease in locomotion was observed when Motor0 was administered 5 minutes after methamphetamine administration condition relative to when D5W was administered 5 minutes later (paired t-test, t(14)=8.282, p=0.0001). Although not directly comparable from an experimental design perspective, importantly locomotion levels in the 5-minute reversal using Motor0 closely approximate those observed in control conditions on Day 1-7, while locomotion counts in the D5W condition appear to approximate those observed with the methamphetamine only treatment. Collectively these findings suggest that Motor0 is capable of sequestering methamphetamine in vivo and reversing methamphetamine-induced hyperlocomotion, with little to no effect on the locomotor behavior of the animal itself.

### Example 3

This example provides a description of in vivo activity of compounds of the present disclosure.

ITC was used to determine the binding constants for various drugs (FIG. 88) to host 1. The data are found in Table 2.

TABLE 2
Binding constants measured by ITC for host guest
complexes of 1 Conditions: 20 mM sodium phosphate
buffered H₂O, pH 7.4, 25° C.

| | Kₐ [M⁻¹]; ΔH (kcal mol⁻¹) |
|---|---|
| | 1 |
| Ketamine | 8.13 × 10³; −12.1 |
| Hydromorphone | 2.54 × 10⁶; −15.4 |
| Heroin | 8.20 × 10⁵; −14.5 |
| Oxycodone | 3.24 × 10⁶; −19.4 |
| Hydrocodone | 2.88 × 10⁶; −18.5 |
| MDMA | 2.54 × 10⁶; −17.8 |
| Mephedrone | 1.24 × 10⁶; −16.4 |
| Meperidine | 9.09 × 10³; −9.08 |

Although the present disclosure has been described with respect to one or more particular embodiments and/or examples, it will be understood that other embodiments and/or examples of the present disclosure may be made without departing from the scope of the present disclosure.

1. A compound having the following structure: 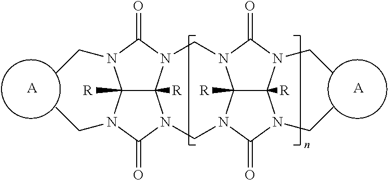
wherein each R is independently a hydrogen, a C₁ to C₂₀ alkyl group, a C₃ to C₂₀ carbocyclic group, a C₁ to C₂₀ heterocyclic group, a carboxylic acid group, an ester group, an amide group, a hydroxy, or an ether group;
wherein, optionally, adjacent R groups form a C₃ to C₂₀ carbocyclic ring or heterocyclic ring;
wherein each 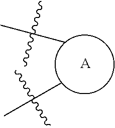
is independently a C₅ to C₂₀ carbocyclic ring system or C₂ to C₂₀ heterocyclic ring system, wherein the ring system comprises one or more rings;
wherein at least one ring system has at least one ionizable group selected from —OS(O)₂O⁻M⁺ and —OS(O)₂OH,
wherein M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, and tris(hydroxymethyl)aminomethane (TRIS), and
wherein n is 0 to 6,
or a stereoisomer or mixtures thereof, a salt, a partial salt, a hydrate, a polymorph or a mixture thereof.

2. The compound of claim 1, wherein each 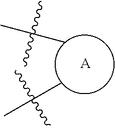
is independently a C₅ to C₂₀ carbocyclic ring having one of the following structures: 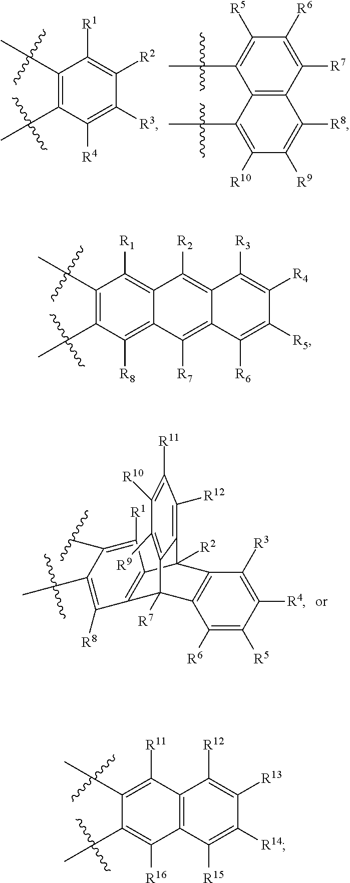
wherein at each occurrence of 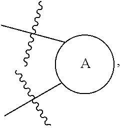
R¹ to R¹⁶ is independently chosen from hydrogen, —OS(O)₂O⁻M⁺, —OS(O)₂OH, non-sulfate anionic groups, carboxylic acid/carboxylate groups, phosphonic acid/phosphonate groups, phosphate groups, substituted or unsubstituted aryl groups, substituted or unsubstituted heteroaryl groups, substituted or unsubstituted aliphatic groups, O-alkyl groups, azide groups,
wherein at least one of R¹ to R¹⁶ is —OS(O)₂O⁻M⁺ or —OS(O)₂OH.

3. The compound of claim 2, wherein the 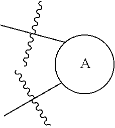
groups are the same.

4. The compound of claim 2, wherein the 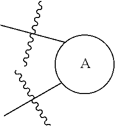
groups are

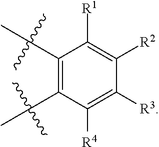

5. The compound of claim 4, wherein R¹ and R⁴ are —OS(O)₂O⁻M⁺.

6. The compound of claim 1, wherein n is 3.

7. The compound of claim 1, wherein each R is independently hydrogen or methyl.

8. The compound of claim 4, wherein R² and R³ are hydrogen.

9. The compound of claim 1, wherein the compound has the following structure: 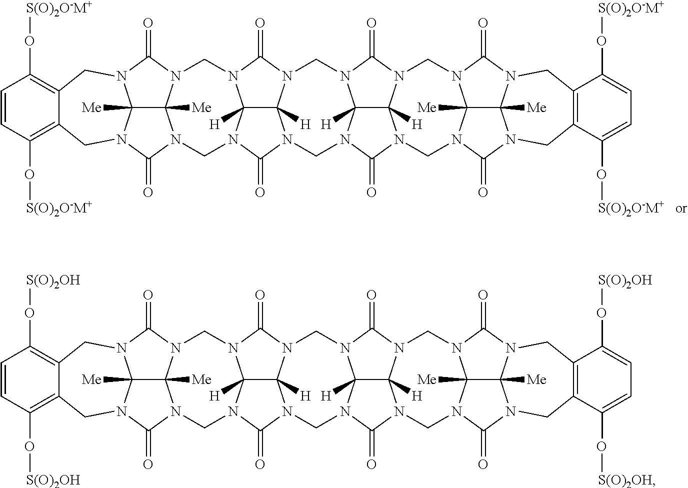
or a stereoisomer or mixtures thereof, a salt, a partial salt, a hydrate, a polymorph or a mixture thereof.

10. The compound of claim 9, wherein M⁺is Na⁺, K⁺, H₄N⁺, Et₃NH⁺, Me₄N⁺, (HOCH₂CH₂)₃NH⁺.

11. The compound of claim 10, wherein M⁺ is Na⁺.

12. A composition comprising one or more compound(s) according to claim 1.

13. The composition of claim 12, further comprising a pharmaceutical carrier.

14. The composition of claim 12, further comprising a pharmaceutical agent.

15. The composition of claim 14, wherein the pharmaceutical agent is non-covalently complexed to the compound.

16. The composition of claim 14, wherein the pharmaceutical agent has a solubility of less than 100 μM in an aqueous solvent.

17. The composition of claim 12, wherein the one or more compound(s) is disposed to at least a portion of a solid substrate.

18. The composition of claim 17, wherein the solid substrate comprises silica, polymer beads, polymer resins, metal nanoparticles, or a metal.

19. The composition of claim 17, wherein at least a portion (or all) of the one or more compound(s) have one or more pharmaceutically active agent(s) non-covalently bound thereto.

20. A method for sequestering one or more neuromuscular blocking agent(s), one or more anesthesia agent(s), one or more pharmaceutical agent(s), one or more pesticide(s), one or more dyestuff(s), one or more malodorous compound(s), one or more chemical warfare agent(s), one or more hallucinogen(s), one or more toxin(s), one or more metabolite(s), or a combination thereof comprising:
contacting the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof with one or more compound(s) of claim 1, wherein the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof are sequestered by the one or more compound(s).

21. The method of claim 20, wherein the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof is present in an aqueous sample, in a solid sample, in a gas sample, or on a solid surface.

22. The method of claim 21, wherein the aqueous sample is a wastewater sample, an industrial water sample, or a municipal water sample.

23. The method of claim 20, wherein a complex is formed from the one or more compound(s) and the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof.

24. The method of claim 20, wherein the complex is removed from the aqueous sample, the solid sample, or the gas sample.

25. The method of claim 20, wherein the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof is present in and/or on an individual and the contacting comprises administration of the one or more compound(s).

26. The method of claim 25, wherein the individual is a human or a non-human mammal.

27. A method for reversing drug-induced neuromuscular block and/or anesthesia and/or the effects of one or more pharmaceutical agent(s) in an individual comprising administering to an individual in need of reversal of neuromuscular block and/or reversal of anesthesia and/or reversal of the effects of one or more pharmaceutical agent(s) one or more compound(s) of claim 1.

28. The method of claim 27, wherein the individual is in need of reversal of drug-induced neuromuscular block.

29. The method of claim 27, wherein the individual is in need of reversal of anesthesia.

30. The method of claim 27, wherein the individual is in need of reversal of drug-induced neuromuscular block and anesthesia.

31. The method of claim 27, wherein the individual is in need of reversal of the effects of one or more one or more pharmaceutical agent(s), wherein the one or more pharmaceutical agent(s) are chosen from one or more drug(s) of abuse, one or more pesticide(s), one or more chemical warfare agent(s), one or more nerve agent(s), one or more hallucinogen(s), one or more toxin(s), one or more metabolite(s), and combinations thereof.

32. The method of claim 27, wherein the individual in need is a human.

33. The method of claim 27, wherein the individual in need is a non-human mammal.

34. A method for prophylaxis and/or therapy of a condition in an individual comprising administering to an individual in need of the prophylaxis and/or the therapy a composition comprising a compound of claim 1, wherein subsequent to the administration the therapy and/or the prophylaxis of the condition in the individual occurs.

35. A method for prophylaxis and/or therapy of a condition in an individual comprising administering to an individual in need of the prophylaxis and/or the therapy (i) one or more compound(s) of claim 1, and (ii) one or more pharmaceutical agent(s), wherein the compound(s) and the pharmaceutical agent(s) are present as complex,
wherein subsequent to the administration the therapy and/or the prophylaxis of the condition in the individual occurs.

36. The method of claim 35, wherein one or more of the pharmaceutical agent(s) has/have a solubility of less than 100 μM in an aqueous solvent.
